(19) United States
(12) Patent Application Publication
(10) Pub. No.: US 2015/0259331 A1
(43) Pub. Date: Sep. 17, 2015

(54) PROTOZOAN PARASITE GROWTH INHIBITORS

(71) Applicant: Northeastern University, Boston, MA (US)

(72) Inventors: Michael P. POLLASTRI, Waltham, MA (US); Naimee MEHTA, Jamaica Plain, MA (US); William DEVINE, Malden, MA (US); Jennifer WOODRING, Brighton, MA (US); Uma SWAMINATHAN, Auburndale, MA (US)

(21) Appl. No.: 14/660,662

(22) Filed: Mar. 17, 2015

Related U.S. Application Data
(60) Provisional application No. 61/954,841, filed on Mar. 18, 2014.
(60) Provisional application No. 61/954,229, filed on Mar. 17, 2014.

(51) Int. Cl. C07D 413/10; C07D 409/04; C07D 401/04; C07D 405/04; C07D 239/94

(52) U.S. Cl. CPC C07D 413/10; C07D 405/04; C07D 239/94; C07D 401/04; C07D 409/04

(57) ABSTRACT

Compounds and methods for inhibiting growth of a protozoan parasite. Methods of treating a protozoan parasite infection in a subject by administering a therapeutically effective amount of a compound as disclosed herein. The compounds and methods can be used to inhibit growth of protozoan parasites such as Trypanosoma brucei, Trypanosoma cruzi, Leishmania spp., and Plasmodium spp.

### CROSS REFERENCE TO RELATED APPLICATIONS

This application claims the benefit of co-pending U.S. Provisional Application No. 61/954,229, filed Mar. 17, 2014 and U.S. Provisional Application No. 61/954,841, filed Mar. 18, 2014, each disclosure of which is hereby incorporated by reference in its entirety.

### STATEMENT REGARDING FEDERALLY SPONSORED RESEARCH/DEVELOPMENT

The present invention was made with United States government support under Grant No. R01Al082577 awarded by the National Institute of Health and under Grant DGE0965843 awarded by the National Science Foundation. The United States government has rights in this invention.

### BACKGROUND

The present application relates to various compounds and the use of the compounds as parasite growth inhibitors. In accordance with certain embodiments, the compounds are used to treat Neglected tropical diseases (NTDs).

Neglected tropical diseases (NTDs) represent a significant global health burden, particularly in developing regions of the world. Estimates of as many as one in six in the world population (over 1 billion people) are infected by one or more NTDs, with one in three people at risk. These diseases are “neglected” because so few research dollars are invested in treating or preventing them, in comparison to those conditions primarily affecting the developed world.

Trypanosoma brucei (which causes human African trypanosomiasis (HAT), Trypanosoma cruzi (Chagas' disease), Leishmania spp. (causative agents for leishmaniases), and Plasmodium spp. (malaria) all express kinases and phosphodiesterases (PDEs) that are involved in aspects of cellular signaling. T. brucei expresses over 180 protein kinases, some of which (such as glycogen synthase kinase-3, phosphoinositol-3-kinases/TOR and Aurora kinase) have been targeted in drug discovery efforts already. There is unequivocal chemical data for protein Tyr phosphorylation in the parasite. However, trypanosomes do not express receptor tyrosine kinases (RTKs) and it is widely held that Tyr-phosphorylation must therefore be performed by dual-specificity enzymes (e.g., weel) that act on Ser/Thr as well as Tyr residues.

Most drugs for these diseases are considered to be unsuitable because of insufficient efficacy, toxicity, prohibitive cost, an/or increasing drug resistance. There is a need for new drugs with improved physiochemical properties for use in the treatment of these diseases.

### SUMMARY

According to aspects of the present disclosure, a compound represented by the following structure:

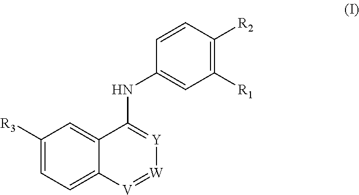

wherein

V, W and Y are independently CH or N;

R₁ is hydrogen, halogen or —OMe;

R₂ is hydrogen, —(C₁-C₆)-alkyl, —OR₄ ; or R₁ and R₂ together form a 3 to 8-membered heterocycle, wherein at least one of the ring carbon atoms is optionally replaced with a heteroatom, selected from the group consisting of N, O and S, and wherein the heterocycle is optionally substituted;

R₃ is substituted or unsubstituted 6 member aryl or heterocycle; and

R₄ is H, —(C₁-C₆)-alkyl, benzyl, substituted benzyl, halo-, dihalo-, or trihalo benzyl, methoxybenzyl or a pharmaceutically acceptable salt thereof is disclosed.

In accordance with certain aspects, the compound may be represented by the following structure:

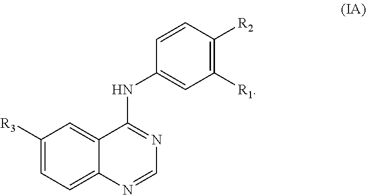

In accordance with other aspects, the compound may be represented by the following structure:

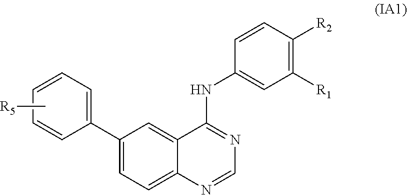

wherein R₅ is hydrogen, —(C₁-C₆)-alkyl, —(C₃-C₅)-cycloalkyl, —C(O)R₆, or —S(O)₂R₆, and R₆ is —(C₁-C₆)-alkyl, aminoalkyl, of a 3 to 8-membered heterocycle, wherein at least one of the ring carbon atoms is optionally replaced with a heteroatom (N, O, or S), and wherein the heterocycle is optionally substituted with —(C₁-C₆)-alkyl.

In yet other aspects, the compound may be represented by the following structure:

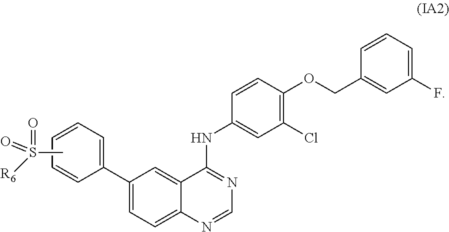

In certain aspects, R₆ in structure IA2 may be CH₃;

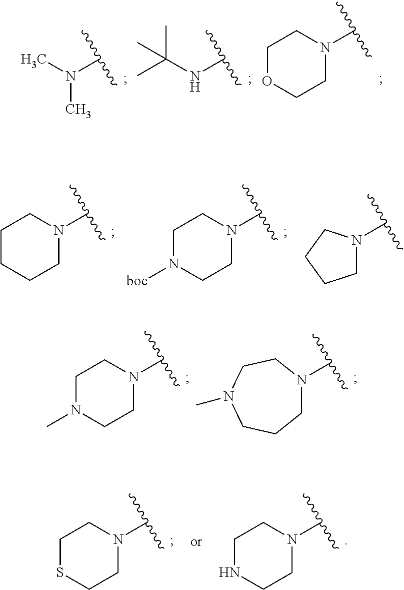

Compounds disclosed herein may also be represented by the following structure:

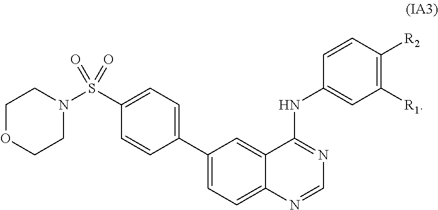

In accordance with other aspects, the compound may be represented by the following structure:

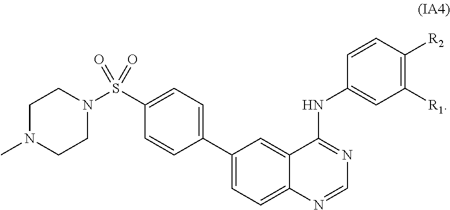

In accordance with some aspects, the compound is:

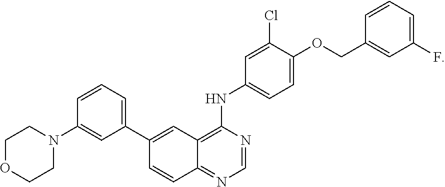 [SMILES: Fc1cccc(COc2ccc(Nc3ncnc4ccc(-c5cccc(N6CCOCC6)c5)cc34)cc2Cl)c1]

The present application is also directed to compounds represented by the following structure:

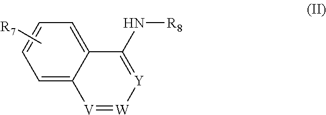

wherein

V, W and Y are independently CH or N, wherein at least 1 of V, W and Y is N;

R₇ is substituted or unsubstituted aryl; and

R8 is substituted or unsubstituted aryl or 5 to 6-membered heterocycle, wherein at least one of the ring carbon atoms is optionally replaced with a heteroatom, selected from N, O and S and wherein the heterocycle is optionally substituted, or a pharmaceutically acceptable salt thereof.

In accordance with certain aspects, the compound may be represented by the following structure:

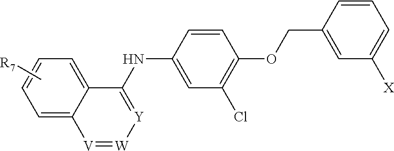

wherein X is hydrogen or halogen. In certain embodiments, the V is N and W and Y are each CH.

In accordance with certain aspects, R₇ is:

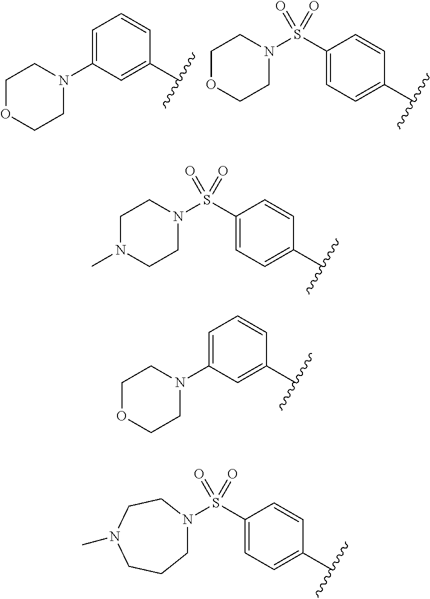

In accordance with other embodiments, the present application discloses a composition comprising a compound as described herein and a pharmaceutically acceptable carrier.

Methods of treating protozoan parasite infection in a subject comprising administration of a therapeutically effective amount of a compound disclosed herein are also provided. In accordance with particular embodiments, the protozoan parasite is selected from the group consisting of Trypanosoma brucei, Trypanosoma cruzi, Leishmania spp., and Plasmodium spp. Methods for inhibiting growth of a protozoan parasite are also provided.

### BRIEF DESCRIPTION OF THE FIGURES

FIG. 1 shows the pharmacokinetics results for 23a (NEU617), following 40 mg/kg oral dosage in male Balb/c mice. The EC₅₀ of NEU617 is 0.042 μM, or 22.7 ng/mL.

FIG. 2 provides individual brain concentration (ng/mL)-time data of NEU-617 (23a) following a single oral administration in male BALB/c mice (Dose: 40 mg/kg).

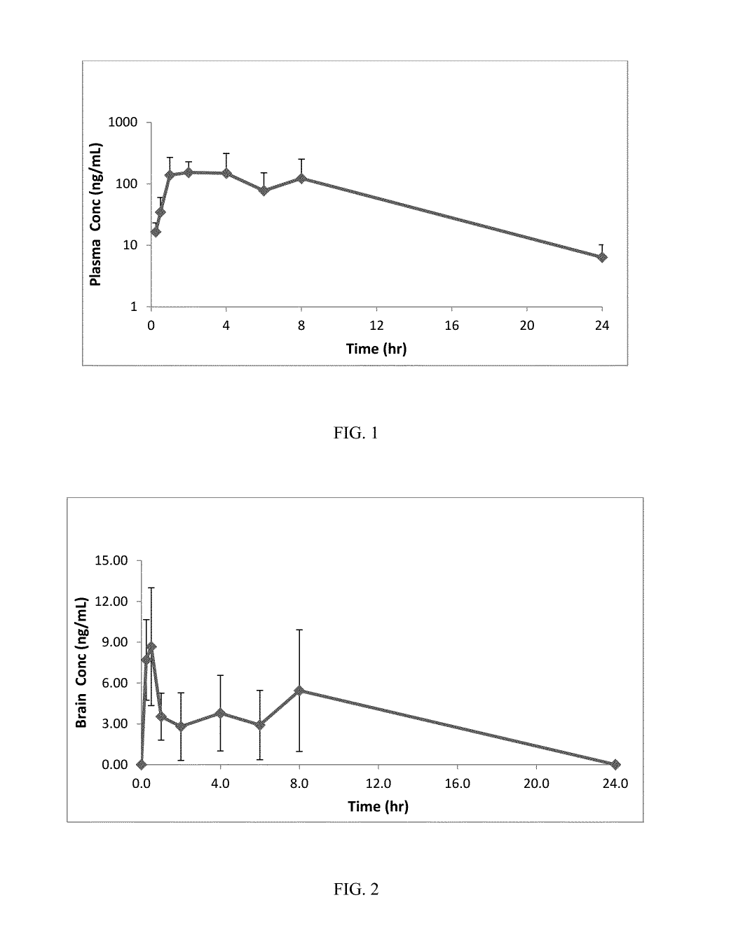

### DETAILED DESCRIPTION OF THE INVENTION

Definitions

The following are definitions of terms used in the present specification. The initial definition provided for a group or term herein applies to that group or term throughout the present specification individually or as part of another group, unless otherwise indicated.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. The term “(C₁-C₆)alkyl” refers to a straight or branched chain alkane (hydrocarbon) radical containing from 1 to 6 carbon atoms, such as n-hexyl, 2-methylpentyl, 3-methylpentyl, 4-methylpentyl, 2,3-dimethylbutyl, 2,2-dimethylbutyl, in addition to those exemplified for “(C₁-C₄)alkyl.” “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplaries of such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “alkynyl” refers to a straight or branched chain hydrocarbon radical containing from 2 to 12 carbon atoms and at least one carbon to carbon triple bond. An exemplary of such groups includes ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{b}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplaries of such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂0Rₑ, NR_{b}Rₑ, NR_{b}S(═O)₂Rₑ, NR_{b}P(═O)₂Rₑ, S(═O)₂NR_{b}Rₑ, P(═O)₂NR_{b}Rₑ, C(═O)OR_{d}, C(═O)Rₐ, C(═O)NR_{b}Rₑ, OC(═O)Rₐ, OC(═O)NR_{b}Rₑ, NR_{b}C(═O)ORₑ, NR_{d}C(═O)NR_{b}Rₑ, NR_{d}S(═O)₂NR_{b}R_{c}, NRA═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}Rₑ, NR_{b}S(═O)₂Rₑ, NR_{b}P(═O)₂Rₑ, S(═O)₂NR_{b}Rₑ, P(═O)₂NR_{b}Rₑ, C(═O)OR_{d}, C(═O)Rₐ, C(═O)NR_{b}Rₑ, OC(═O)Rₐ, OC(═O)NR_{b}Rₑ, NR_{b}C(═O)ORₑ, NR_{d}C(═O)NR_{b}R_{c}, NR_{d}S(═O)₂NR_{b}Rₑ, NR_{d}P(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include fused cylic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “alkylamino” refers to a group having the structure —NHR′, wherein R′ is hydrogen, alkyl or substituted alkyl, cycloalkyl or substituted cyclolakyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocylyl or substituted heterocyclyl, as defined herein. R and R′ may be the same or different in an dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine or iodine.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of a compound of the present invention may be formed, for example, by reacting a compound I with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

Compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% pure (“substantially pure” compound I), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

A “pharmaceutical composition” refers to a mixture of one or more of the compounds described herein, or pharmaceutically acceptable salts, solvates, or hydrates thereof, with other chemical components, such as physiologically acceptable carriers and excipients. The purpose of a pharmaceutical composition is to facilitate administration of a compound to an organism or subject.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety).

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

### Compounds

The present application is directed to various compounds and methods of treating protozoan parasite infection in a subject comprising administration of a therapeutically effective amount of a compound as disclosed herein. In accordance with particular embodiments, the protozoan parasite is selected from the group consisting of Trypanosoma brucei, Trypanosoma cruzi, Leishmania spp., and Plasmodium spp. Methods for inhibiting growth of a protozoan parasite are also provided.

According to aspects of the present disclosure, a compound represented by the following structure:

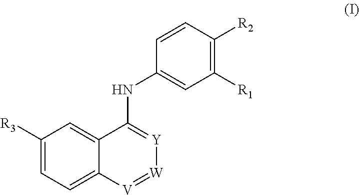

wherein

V, W and Y are independently C or N;

R₁ is hydrogen, halogen or —OMe;

R₂ is hydrogen, —(C₁-C₆)-alkyl, —OR₄; or R₁ and R₂ together form a 3 to 8-membered heterocycle, wherein any one of the ring carbon atoms is optionally replaced with a heteroatom, and wherein the heterocycle is optionally substituted;

R₃ is substituted or unsubstituted 6 member aryl or heterocycle; and

R₄ is H, —(C₁-C₆)-alkyl, benzyl, substituted benzyl, halo-, dihalo-, or trihalo benzyl, methoxybenzyl or a pharmaceutically acceptable salt thereof is disclosed.

In accordance with certain aspects, the compound may be represented by the following structure:

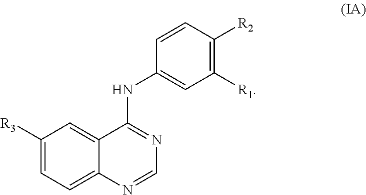

In accordance with other aspects, the compound may be represented by the following structure:

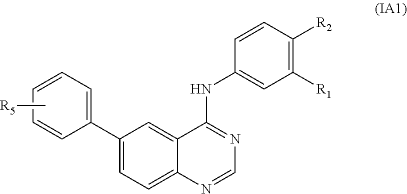

wherein R₅ is hydrogen, —(C₁-C₆)-alkyl, —(C₃-C₅)-cycloalkyl, —C(O)R₆, or —S(O)₂R₆, and R₆ is —(C₁-C₆)-alkyl, aminoalkyl, of a 3 to 8-membered heterocycle, wherein any one of the ring carbon atoms is optionally replaced with a heteroatom, and wherein the heterocycle is optionally substituted with —(C₁-C₆)-alkyl.

In yet other aspects, the compound may be represented by the following structure:

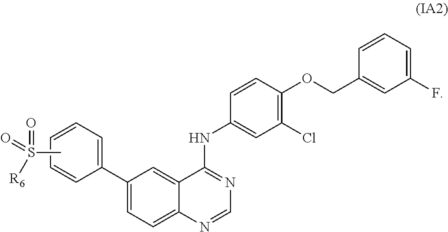

In certain aspects, R₆ in structure IA2 may be CH₃;

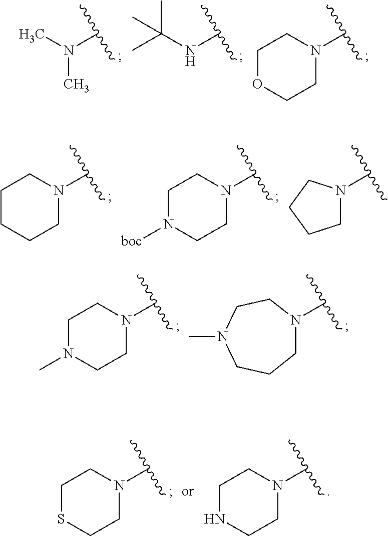

Compounds disclosed herein may also be represented by the following structure:

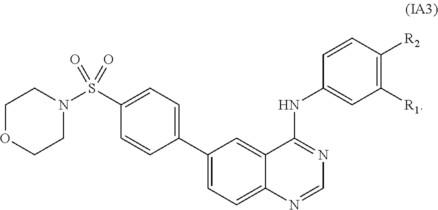

In accordance with other aspects, the compound may be represented by the following structure:

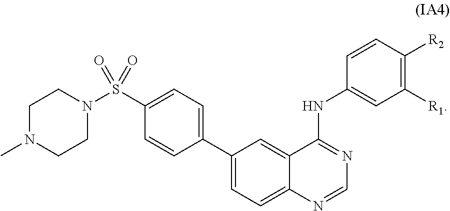

In accordance with some aspects, the compound is:

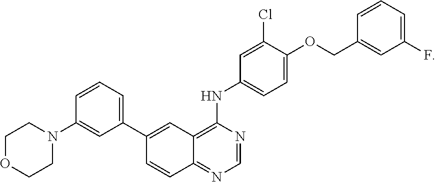 [SMILES: Fc1cccc(COc2ccc(Nc3ncnc4ccc(-c5cccc(N6CCOCC6)c5)cc34)cc2Cl)c1]

In accordance with other embodiments, the present application discloses a composition comprising a compound as described herein and a pharmaceutically acceptable carrier.

Methods of treating protozoan parasite infection in a subject comprising administration of a therapeutically effective amount of a compound disclosed herein are also provided. In accordance with particular embodiments, the protozoan parasite is selected from the group consisting of Trypanosoma brucei, Trypanosoma cruzi, Leishmania spp., and Plasmodium spp. Methods for inhibiting growth of a protozoan parasite are also provided.

Although the compounds described herein have been exemplified based on a quinaz heterocycle core, the same substituents and description applies to other heterocycle cores, such as quinolines, isoquinolines, cinnolines, and phthalazines.

Nine quinazoline-based EGFR inhibitors (1-9, Table 1) from GlaxoSmithKline were screened against cultures of T. brucei brucei Lister 427.

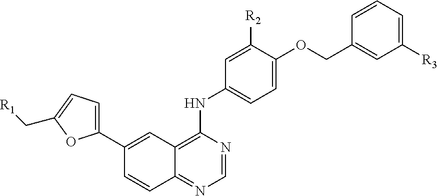

TABLE 1

| | | | | | | | Tbb |
|---|---|---|---|---|---|---|---|
| Entry | Compd | GSK Number | NEU Number | R₁ | R₂ | R₃ | EC ₅₀ (μM)^{a,b} |
| 1 | 2 | GW58337A | NEU-0000382 | 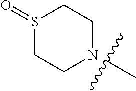 | Cl | H | 0.41 |
| 2 | 3 | GW601906A | NEU-0000387 | 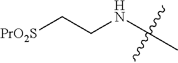 | Cl | F | 0.43 |
| 3 | 4 | GW633460A | NEU-0000379 | 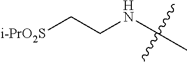 | Cl | F | 0.48 |
| 4 | 5 | GW616030X | NEU-0000381 | 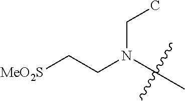 [SMILES: *N(CC)CCS(C)(=O)=O] | Cl | F | 0.52 |
| 5 | 6 | GW615311X | NEU-0000380 | 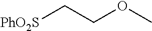 | Cl | F | 0.55 |
| 6 | 7 | GW580496A | NEU-0000383 | 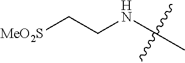 [SMILES: *NCCS(C)(=O)=O] | Br | H | 0.56 |
| 7 | 8 | GW576924A | NEU-0000386 | 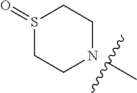 | F | F | 0.60 |
| 8 | 9 | GW616907X | NEU-0000388 | 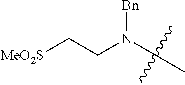 [SMILES: *N(CCS(C)(=O)=O)Cc1ccccc1] | Cl | F | 1.51 |
| 9 | 1 | lapatinib | NEU-0000378 | 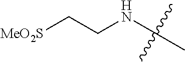 [SMILES: *NCCS(C)(=O)=O] | Cl | F | 1.54 |
| | | melarsoprol | | | | | 0.0063 |
| | | eflornithine | | | | | 16.4 |
| | | pentamidine | | | | | 0.0035 |
| | | SCYX-7158 | | | | | 0.794 |

^{a}All EC₅₀ values are ±7%.
^{b}Concentration giving 50% inhibition of growth of T brucei brucei Lister 427 cells

The inhibitors demonstrated a 4-fold range in potency (Table 1). Also included in Table 1 the published activities of three front-line HAT treatments (eflornithine, melarsoprol, and pentamidine) and of SCYX-7158, currently in clinical trials.

Replacements for the furan-derived tail were evaluated through a broad diversity scan utilizing Suzuki chemistry methodology using boronic acids or esters to enumerate a virtual library of analogues of lapatinib (Scheme 1).

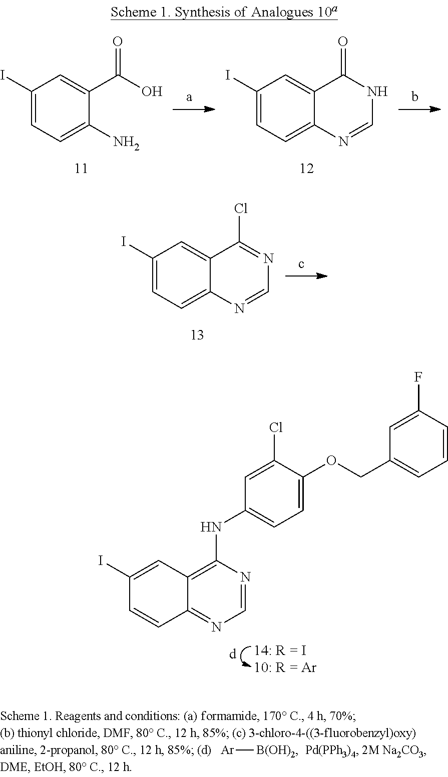

In anticipation of parallel synthesis, iodoquinazoline 14 was prepared by the route shown in Scheme 1. Treatment of the commercially available anthranilic acid 11 with formamide proceeded in 70% yield, followed by chlorination with thionyl chloride to provide the chloroquinazoline 13 in 85% yield. This template was reacted with the requisite aniline (17, Scheme 2), which was prepared by a sequence of alkylation of the nitrophenol.

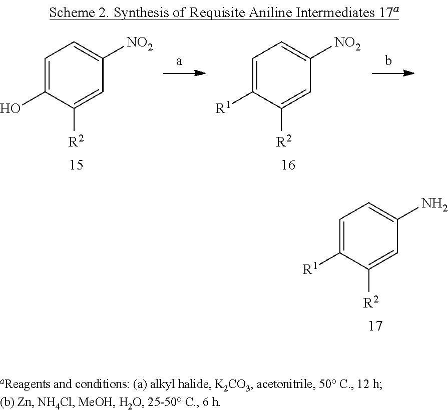

15 with 3-fluorobenzyl bromide followed by nitro group reduction. With the required template 14 in hand, 10 analogues (10a-j) were prepared from the selected boronates using standard Suzuki reaction conditions. The structures and biological activities for these compounds are summarized in Table 2.

TABLE 2

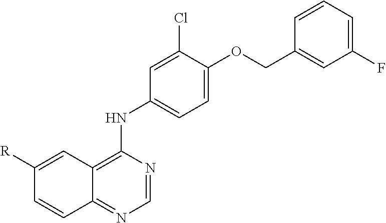

| Com- | | | Tbb EC₅₀ |
|---|---|---|---|
| pound | NEU | R₁ | (μM)^{a,b} |
| 10a | NEU-0000369 | 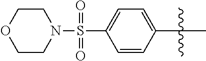 [SMILES: *c1ccc(S(=O)(=O)N2CCOCC2)cc1] | 1.39 |
| 10b | NEU-0000373 | 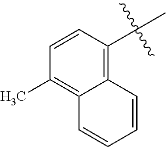 [SMILES: *c1ccc(C)c2ccccc12] | 2.27 |
| 10c | NEU-0000375 | 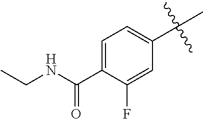 [SMILES: *c1ccc(C(=O)NCC)c(F)c1] | 3.85 |
| 10d | NEU-0000370 | 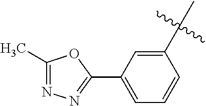 [SMILES: *c1cccc(-c2nnc(C)o2)c1] | 4.21 |
| 10e | NEU-0000376 | 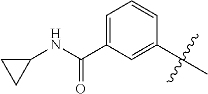 [SMILES: *c1cccc(C(=O)NC2CC2)c1] | 4.50 |
| 10f | NEU-0000366 | 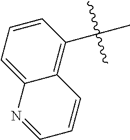 [SMILES: *c1cccc2ncccc12] | 4.53 |
| 10g | NEU-0000372 | 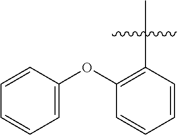 [SMILES: *c1ccccc1Oc1ccccc1] | 5.3 |
| 10h | NEU-0000371 | 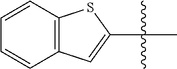 [SMILES: *c1cc2ccccc2s1] | 5.98 |
| 10i | NEU-0000374 | 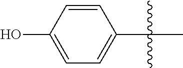 [SMILES: *c1ccc(O)cc1] | 6.45 |
| 10j | NEU-0000367 | 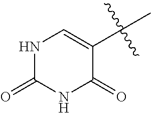 [SMILES: *c1c[nH]c(=O)[nH]c1=O] | 22.63 |

^{a}All EC₅₀ values are ±7%.
^{b}Concentration giving 50% inhibition of growth of T brucei brucei Lister 427 cells

From this series of analogues, NEU369 (10a) was identified as being approximately equipotent to 1 against T brucei cells. Further testing of this compound and its analogues showed that, unlike 1, it did not inhibit HepG2 cell growth (EC50>15 μM) (Table 3).

TABLE 3

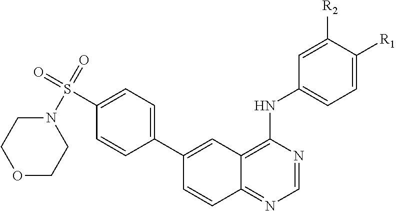

| | | | | | Tbb EC₅₀ | HepG2 IC₅₀ |
|---|---|---|---|---|---|---|
| Entry | Compd | NEU | R₁ | R₂ | (μM)^{a,b} | uM) |
| 1 | 10a | NEU-0000369 | 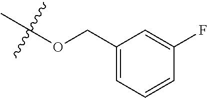 [SMILES: *OCc1cccc(F)c1] | Cl | 1.39 | >15 |
| 2 | 20a | NEU-0000548 | H | H | 1.44 | >3 |
| 3 | 20b | NEU-0000549 | CH₃ | H | 1.15 | >15 |
| 4 | 20c | NEU-0000550 | OH | Cl | 1.06 | >15 |
| 5 | 20d | NEU-0000555 | OCH₃ | Cl | 0.82 | >15 |
| 6 | 20e | NEU-0000551 | 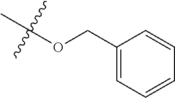 [SMILES: *OCc1ccccc1] | Cl | 1.35 | >15 |
| 7 | 20f | NEU-0000552 | 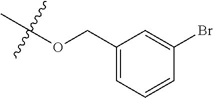 [SMILES: *OCc1cccc(Br)c1] | Cl | 0.68 | >15 |
| 8 | 20g | NEU-0000553 | 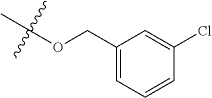 [SMILES: *OCc1cccc(Cl)c1] | Cl | 0.66 | >15 |
| 9 | 20h | NEU-0000564 | 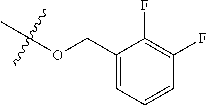 [SMILES: *OCc1cccc(F)c1F] | Cl | 0.82 | >15 |
| 10 | 20i | NEU-0000565 | 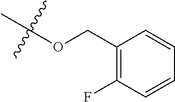 [SMILES: *OCc1ccccc1F] | Cl | 1.65 | >15 |
| 11 | 20j | NEU-0000566 | 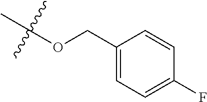 [SMILES: *OCc1ccc(F)cc1] | Cl | 1.34 | >15 |
| 12 | 20k | NEU-0000567 | 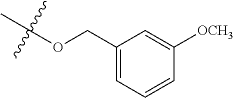 [SMILES: *OCc1cccc(OC)c1] | Cl | 1.43 | >15 |
| 13 | 20l | NEU-0000568 | 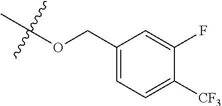 [SMILES: *OCc1ccc(C(F)(F)F)c(F)c1] | Cl | 0.54 | >15 |
| 14 | 20m | NEU-0000569 | 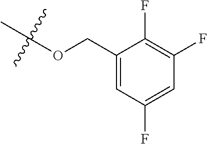 [SMILES: *OCc1cc(F)cc(F)c1F] | Cl | 1.12 | >3 |
| 15 | 20n | NEU-0000554 | 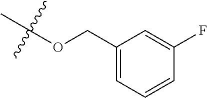 [SMILES: *OCc1cccc(F)c1] | H | 0.65 | >15 |
| 16 | 20o | NEU-0000570 | 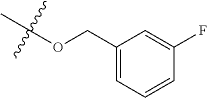 [SMILES: *OCc1cccc(F)c1] | OCH₃ | 1.88 | >15 |

^{a}All EC₅₀ values are ±7%.
^{b}Concentration giving 50% inhibition of growth of T brucei brucei Lister 427 cells

Keeping the newly identified tail group present in 10a (Table 3), the aniline headgroup region of the molecule was explored. Preparation of the requisite chloroquinazoline 19 (Scheme 3) was achieved by treatment of 12 with the required boronic acid using Suzuki conditions, followed by chlorination with thionyl chloride. This intermediate was reacted with a range of anilines (Scheme 2) to provide analogues 20 (Table 3).

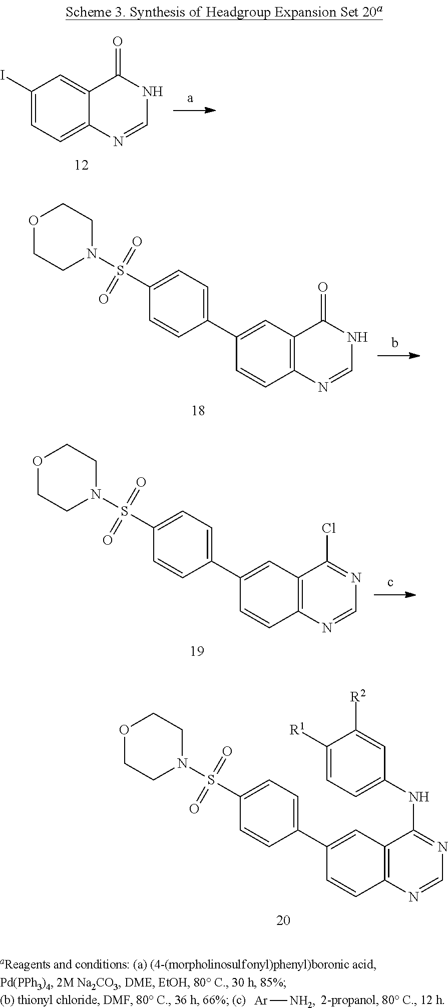

This library was designed to explore the role of halogen substitutions on the terminal benzyl substituent (R1) headgroup, testing positional isomers of fluoro substitutions and other potential halogen replacements such as methoxy and trifluoromethyl groups. These modifications produced insignificant changes in activity of the compounds against T. brucei. A few analogues were prepared to assess functional group tolerance at the R2 position of the headgroup, replacing the chlorine atom of 1 with hydrogen and methoxy groups; these changes also resulted in very modest alterations in antitrypanosomal activity.

Interestingly, truncation of the molecule (20a) gave potency approximately equal to 10a, translating to a similar ligand efficiency value (LE of 10a=0.14; 20a=0.18).For the next round of analogues, further refinement of the tail group region of 1 was explored by performing focused modifications of the 6-aryl position of the quinazoline ring that were designed to evaluate steric requirements as well as the required adornment of polarity in this region of the inhibitor. These compounds (10k-w) were accessed from the corresponding boronic acids using the route shown in Scheme 1. The larger sulfonamide side chains showed some modest preference for meta-substitution (Table 4, entries 1-6), although the methyl sulfones (entries 11-12) showed preference for para-substituents. Comparing the sulfonamide substituents, morpholine was preferred over the other heterocycles tested.

TABLE 4

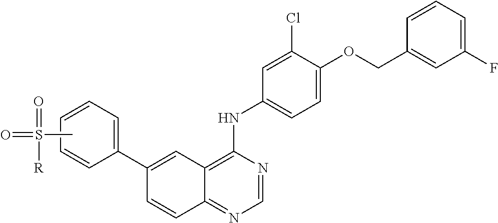

| | | | | | Tbb EC₅₀ | HepG2 IC₅₀ |
|---|---|---|---|---|---|---|
| Entry | Compd | NEU | position | R₁ | (μM)^{a,b} | (uM) |
| 1 | 10a | NEU-0000369 | p | 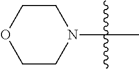 [SMILES: *N1CCOCC1] | 1.39 | >15 |
| 2 | 10k | NEU-0000621 | m | 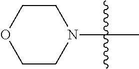 [SMILES: *N1CCOCC1] | 0.33 | >15 |
| 3 | 10l | NEU-0000622 | p | 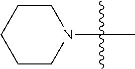 [SMILES: *N1CCCCC1] | 0.32 | >15 |
| 4 | 10m | NEU-0000623 | m | 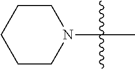 [SMILES: *N1CCCCC1] | 0.46 | >15 |
| 5 | 10n | NEU-0000625 | p | 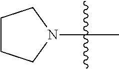 [SMILES: *N1CCCC1] | 0.35 | >15 |
| 6 | 10o | NEU-0000626 | m | 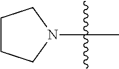 [SMILES: *N1CCCC1] | 0.25 | >15 |
| 7 | 10p | NEU-0000627 | p | 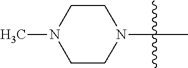 [SMILES: *N1CCN(C)CC1] | 0.53 | >15 |
| 8 | 10q | NEU-0000628 | p | 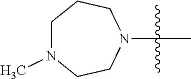 [SMILES: *N1CCCN(C)CC1] | 0.81 | 1.81 |
| 9 | 10r | NEU-0000629 | p | 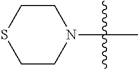 [SMILES: *N1CCSCC1] | 0.47 | >15 |
| 10 | 10s | NEU-0000630 | p | 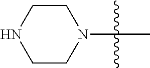 [SMILES: *N1CCNCC1] | 0.28 | 3.32 |
| 11 | 10t | NEU-0000631 | p | CH₃ | 0.90 | >15 |
| 12 | 10u | NEU-0000633 | m | CH₃ | 3.21 | >15 |
| 13 | 10v | NEU-0000619 | o | N(CH₃)₂ | 1.04 | >15 |
| 14 | 10w | NEU-0000620 | o | NHC(CH₃)₃ | 4.66 | nd^{c} |

^{a}All EC₅₀ values are ±7%.
^{b}Concentration giving 50% inhibition of growth of T brucei brucei Lister 427 cells.
^{c}Not determined.

A more focused evaluation of linker and regiochemistry is shown in Table 5. Compounds were synthesized from 14 by reaction with the appropriate boronate ester 22.

TABLE 5

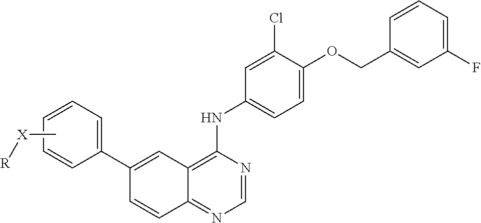

| | | | | | | Tbb EC₅₀ | HepG2 IC₅₀ |
|---|---|---|---|---|---|---|---|
| Entry | Compd | NEU | R | Regio | X | (μM)^{a} | (μM) |
| 1 2 3 4 5 6 | 23a 23b 23c 23d 23e 23f | NEU-0000617 NEU-0000733 NEU-0000786 NEU-0000636 NEU-0000782 NEU-0000783 | 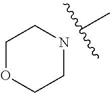 | m p m p m p | — — CH₂ CH₂ C═O C═O | 0.042 1.91 0.36 0.99 0.55 1.21 | >20 9.6 nd^{c} 12.9 nd nd |
| 7 | 10k | NEU-0000621 | | m | SO₂ | 0.33 | >20 |
| 8 | 10a | NEU-0000369 | | p | SO₂ | 1.39 | >20 |
| 9 10 11 12 13 14 | 23g 23h 23i 23j 23k 23l | NEU-0000712 NEU-0000784 NEU-0000785 NEU-0000787 NEU-0000639 NEU-0000638 | 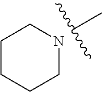 | m p m p m p | — — CH₂ CH₂ C═O C═O | 0.76 0.38 0.65 1.5 0.14 0.94 | >15 nd nd nd >6 nd |
| 15 | 10m | NEU-0000623 | | m | SO₂ | 0.46 | >20 |
| 16 | 10l | NEU-0000622 | | p | SO₂ | 0.32 | >20 |

^{a}All EC₅₀ values are ±7%.
^{b}Concentration giving 50% inhibition of growth of T brucei brucei Lister 427 cells.
^{c}Not determined.

In the case of the morpholinosulfonamides (entries 1-8), meta-substitution is consistently better than para; the most potent analogue, NEU617 (23a), is directly linked to the aromatic ring (Scheme 4).

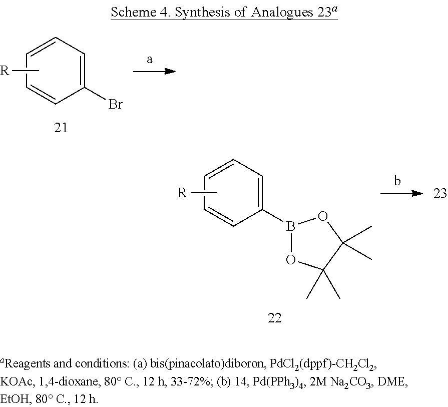

For piperidinosulfonamides (entries 9-16), the meta preference is less consistent, and none of these analogues shows as potent growth inhibition as 23a. When the tail contains a morpholine (entries 1-8), the linker appears to have little impact on potency (except for 23a, a clear outlier); all meta-substituted analogues are otherwise approximately equipotent. In cases where the morpholine moiety is at the para-position, there is little difference resulting from linker variation.

With piperidine-substitution (entries 9-16), it appears that a modest preference exists for the meta-substituted amide, with a 6.7-fold loss of activity when moved to the para position (compare entries 13 and 14), although the importance of positional substitution is otherwise less for other examples, within ˜2-fold in activity. Next, the headgroup substituents of 23a were sequentially removed. Removal of one (23m) or both (23n) halogens afforded an approximately 5-fold reduction in potency, and further truncation (23o) significantly reduced antiparasitic activity while restoring HepG2 potency (Table 6).

TABLE 6

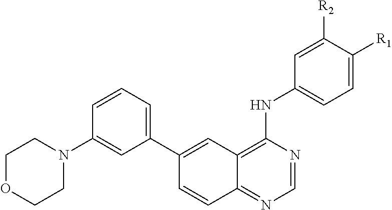

| | | | | Tbb EC₅₀ | HepG2 IC₅₀ | |
|---|---|---|---|---|---|---|
| Compound | NEU | R¹ | R² | (μM)^{a,b} | (μM) | LE |
| 23a | NEU-0000617 | 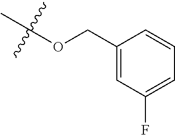 [SMILES: *OCc1cccc(F)c1] | Cl | 0.042 | >20 | 0.19 |
| 23lm | NEU-0000735 | OBn | Cl | 0.23 | >20 | 0.18 |
| 23n | NEU-0000736 | OBn | H | 0.18 | >20 | 0.19 |
| 23o | NEU-0000737 | H | H | 11 | 7.02 | 0.17 |

^{a}All EC₅₀ values are ±7%.
^{b}Concentration giving 50% inhibition of growth of T brucei brucei Lister 427 cells.
^{c}Not determined.

Noting its potency against T. brucei and selectivity over HepG2 cells (Table 5), compound 23a was advanced into a mouse oral pharmacokinetic study. Mice were administered a single oral dose (40 mg/kg) of 23a, and plasma and brain tissue drug levels were measured over 24 h. Although the CNS fraction was low (5%), the plasma levels were in excess of the EC50 for >12 h. It was determined that 23a was 99.6% plasma protein bound. However, because trypanosomes are noted for their ability to endocytose host plasma proteins, it was determined that the oral exposure and in vitro potency warranted in vivo efficacy evaluation.

In a test of efficacy in a mouse model of HAT, mice were infected with T. brucei brucei CA427 (104 cells) and after 24 h were administered a 40 mg/kg dose of 23a once per day. No parasites were detected in the blood of the infected mice for 3 days, whereas control mice had trypanosomes in their blood on day 2 postinfection. However drug-related toxicity was observed with 23a in a multiday regimen at the 40 mg/kg dose. These data point to a need to improve the pharmacokinetic properties of 23a so that it can be more effective in the mouse model of HAT.

The dosing regimen was adjusted, opting to administer 23a at 10 mg/kg twice per day (total dose of 20 mg/kg/day), either orally or intraperitoneally (ip). The results indicated three effects: First, ip treatment with 23a delayed detection of trypanosomes in the blood of infected mice by 24 h. Whereas all control mice had trypanosomes in their blood on day 3, mice treated with 23a all had parasites in their blood 24 h later, suggestive of either a significant reduction in replication rate (trypanosomes divide every 6 h) or of parasite killing during this initial phase of infection. Second, on day 8 when all untreated mice had died, the mice dosed ip with 23a were all alive. In the oral administration experiment, mice died in the same time frame as control mice, suggesting insufficient drug exposure at this dosage. Third, ip administration of 23a led to better control of infection, leading to a doubling of mouse survival life span from 5 days in the control group to 9 days in 23a-treated mice. Although 23a is structurally similar to 1, the biological effects of the two compounds are different. Whereas 1 inhibited endocytosis of transferrin in the trypanosome in a manner similar to what was observed with tyrphostin, 23a had no effect on receptor-mediated endocytosis of transferrin. Instead, 23a affected the cell cycle in ways not observed with 1.Trypanosomes possess two DNA-containing organelles (nucleus and kinetoplast (mitochondrial nucleoid)). The kinetoplast and nucleus can be tracked by microscopy during the cell cycle, which begins with trypanosomes harboring one nucleus (1N) and one kinetoplast (1K) (i.e., 1K1N cells). A kinetoplast that is replicating its (DNA) (i.e., kDNA) and increasing the organelle's mass is observed as an elongated kinetoplast (1Ke). Fission (segregation) of 1Ke kinetoplasts into two daughter kinetoplasts (2K) inside the same cell is coincident with the nuclear S-phase and produces 2K1N trypanosomes. Mitosis then occurs, yielding trypanosomes containing two kinetoplasts and two nuclei (2K2N). Following cell division, each daughter trypanosome has a 1K1N configuration of the organelles. After a 7 h incubation with 23a, the chromosomal DNA profile of T. brucei was altered; the fraction of cells with 2C-4C equivalents of DNA was reduced from 40 to 25%, whereas the proportion of cells with 4C DNA increased from 25% to 40%. Single cell microscopy studies were performed to determine whether the changes in DNA per cell caused by 23a were reflected in alterations in the number of DNA-containing organelles per cell. Quantitation of the data obtained revealed that 23a reduced the number of cells containing one nucleus (i.e., 1K1N, 1Ke1N) but selectively increased the fraction of a group of cells that are not normally detected in the absence of the drug: cells containing two nuclei and one kinetoplast (1K2N). This data is consistent with the increase in trypanosomes with 4C equivalent of DNA. Thus, 23a blocks duplication of the kinetoplast and arrests cytokinesis without inhibiting division of the trypanosome nucleus.

Although compound 23a has high calculated lipophilicity and molecular weight, its oral bioavailability lent the compound to further assessment in a mouse model of HAT, where it provided modest effects in controlling parasitemia, with concomitant life extension of infected mice. Because the pharmacokinetic experiments suggest acceptable plasma levels in mice following oral dosing, it is believed that the high plasma protein binding (99.6%) observed for 23a is the cause of the lower-than-expected effect on in vivo parasite loads. Trypanosome physiology analysis indicates that 23a acts via a mechanism different from 1 and tyrphostin A47.

The following tables provide some additional data for some embodiments of the present invention.

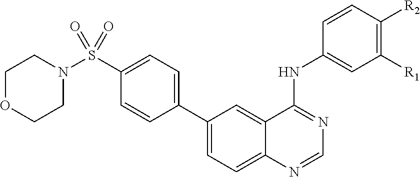

TABLE 7

| | | | | T. | | | | | |
|---|---|---|---|---|---|---|---|---|---|
| | | | T. | cruzi | | | | | |
| | | | brucei | % inh | L. major | | P. falciparum | | |
| | | | EC₅₀ | at 10 | Promastigote | Amastigote | D6 | W2 | C235 |
| Entry | R₁ | R₂ | (μM)^{a} | μM | EC₅₀ (μM)^{b} | EC₅₀ (μM)^{b} | (EC₅₀)^{c} | (EC₅₀)^{c} | (EC₅₀)^{c} |
| NEU- 369 | Cl | 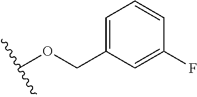 [SMILES: *OCc1cccc(F)c1] | 1.3 | −46 | 1.3 | 1.6 | 0.22 | 0.69 | 0.41 |
| NEU- | H | H | 1.4 | 13 | 4.1 | 4.1 | 4.1 | 4.1 | 4.1 |
| 548 | | | | | | | | | |
| NEU- | H | CH₃ | 1.2 | 59 | 0.45 | >15 | 1.7 | 6.2^{b} | 2.8^{b} |
| 549 | | | | | | | | | |
| NEU- | Cl | OH | 1.1 | 14 | >15 | >15 | 12 | 19 | 19 |
| 550 | | | | | | | | | |
| NEU- | Cl | OBn | 1.4 | 9 | 0.47 | >15 | 0.25 | 0.72 | 0.45 |
| 551 | | | | | | | | | |
| NEU- 552 | Cl | 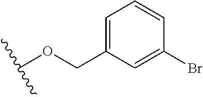 [SMILES: *OCc1cccc(Br)c1] | 0.68 | 7 | 14 | 0.47 | 0.20 | 0.44^{b} | 0.41 |
| NEU- 553 | Cl | 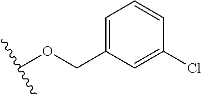 [SMILES: *OCc1cccc(Cl)c1] | 0.66 | 4 | 15 | 1.1 | 0.23 | 0.66 | 0.33 |
| NEU- 554 | H | 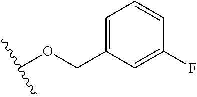 [SMILES: *OCc1cccc(F)c1] | 0.65 | 10 | 0.48 | 0.47 | 0.44 | 2.0 | 0.85 |
| NEU- | Cl | OCH₃ | 0.82 | 43 | 0.83 | 4.8 | 0.81 | 1.7 | 1.2 |
| 555 | | | | | | | | | |
| NEU- 565 | Cl | 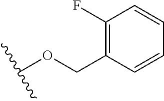 [SMILES: *OCc1ccccc1F] | 1.7 | 10 | 0.71 | >15 | 0.13 | 0.57 | 0.22 |
| NEU- 566 | Cl | 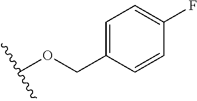 [SMILES: *OCc1ccc(F)cc1] | 1.3 | −1 | 1.7 | >15 | 0.17 | 0.36 | 0.24 |
| NEU- 567 | Cl | 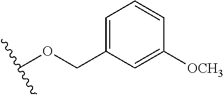 [SMILES: *OCc1cccc(OC)c1] | 1.4 | 5 | 15 | 15 | 0.23 | 0.69^{b} | 0.35 |
| NEU- 568 | Cl | 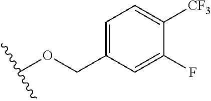 [SMILES: *OCc1ccc(C(F)(F)F)c(F)c1] | 0.54 | 21 | 14 | 2.5 | 0.57 | 1.11 | 0.64 |
| NEU- 569 | Cl | 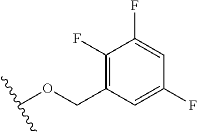 [SMILES: *OCc1cc(F)cc(F)c1F] | 1.1 | 14 | 3.0 | 3.0 | 0.14 | 0.45 | 0.24 |
| NEU- 570 | OCH₃ | 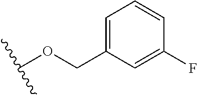 [SMILES: *OCc1cccc(F)c1] | 1.9 | 2 | >15 | >15 | 0.44 | 0.65 | 0.60 |

^{a}All EC₅₀ values are ±7%.
^{b}r² values >0.75.
^{c}All r² values >0.9 unless noted otherwise

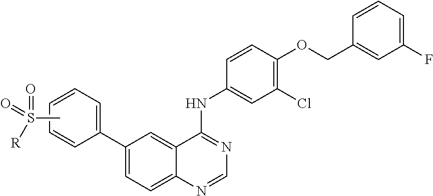

TABLE 8

| | | | T. | T. | | | | | |
|---|---|---|---|---|---|---|---|---|---|
| | | | brucei | cruzi | L. major | | P. falciparum | | |
| | | | EC₅₀ | EC₅₀ | Promastigote | Amastigote | D6 | W2 | C235 |
| Entry | Position | R | (μM)^{a} | (μM)^{b} | EC₅₀ (μM)^{e} | EC₅₀ (μM)^{e} | (EC₅₀)^{f} | (EC₅₀)^{f} | (EC₅₀)^{f} |
| NEU- 619 | o | 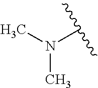 [SMILES: *N(C)C] | 1.0 | 1.5^{d} | 2.8 | >15 | 0.82 | 1.8 | 1.1 |
| NEU- 620 | o | 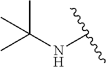 [SMILES: *NC(C)(C)C] | 4.7 | 6.4^{c,d} | ‡ | ‡ | ‡ | ‡ | ‡ |
| NEU- 621 | m | 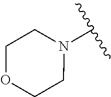 [SMILES: *N1CCOCC1] | 0.33 | 1.7^{c, d} | >15 | >15 | 0.28 | 0.40 | 0.29 |
| NEU- 622 | p | 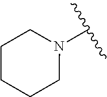 [SMILES: *N1CCCCC1] | 0.32 | (1)* | >15 | >15 | 0.18 | 0.33^{e} | 0.24 |
| NEU- 623 | m | 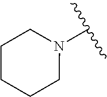 [SMILES: *N1CCCCC1] | 0.46 | >50 | >15 | >15 | 0.33 | 0.59 | 0.35 |
| NEU- 624 | p | 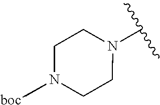 | 1.0 | (5)* | 14 | 14 | 0.27 | 0.58 | 0.43 |
| NEU- 625 | p | 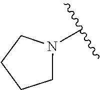 [SMILES: *N1CCCC1] | 0.35 | (10)* | >15 | >15 | 0.20 | 0.80 | 0.42 |
| NEU- 626 | m | 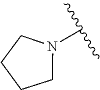 [SMILES: *N1CCCC1] | 0.25 | (32)* | >15 | >15 | 0.19 | 0.48 | 0.30 |
| NEU- 627 | p | 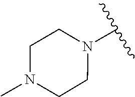 [SMILES: *N1CCN(C)CC1] | 0.53 | (60)* | 2.8 | 1.6 | 0.027 | 0.044 | 0.039 |
| NEU- 628 | p | 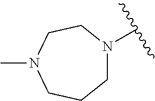 [SMILES: *N1CCCN(C)CC1] | 0.81 | 0.51 | 3.6 | 2.0 | 0.046 | 0.052 | 0.050 |
| NEU- 629 | p | 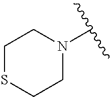 [SMILES: *N1CCSCC1] | 0.47 | (18)* | >15 | >15 | 0.46 | 1.6 | 0.76 |
| NEU- 630 | p | 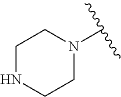 [SMILES: *N1CCNCC1] | 0.28 | 0.79 | >15 | 0.80 | 0.094 | 0.20 | 0.12 |
| NEU- | p | CH₃ | 0.90 | 0.65^{d} | 1.6 | 3.9 | 0.12 | 0.36 | 0.20 |
| 631 | | | | | | | | | |
| NEU- | m | CH₃ | 3.2 | 5^{d} | 3.5 | 4.7 | 0.52 | 1.3 | 0.62 |
| 633 | | | | | | | | | |
| NEU- 770 | m | 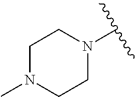 [SMILES: *N1CCN(C)CC1] | 3.3 | 1.4^{d} | >15 | 1.2 | 0.038 | 0.084 | 0.033^{e} |

‡Insoluble under assay conditions.
*% inh at 10 μM.
^{a}All EC₅₀ values are ±7%.
^{b}All SEM values within 35% unless noted otherwise.
^{c}SEM values within 50%.
^{d}n = 2
^{e}r² values >0.75.
^{f}All r² values >0.9 unless noted otherwise

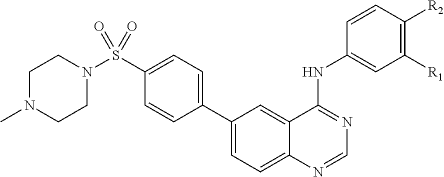

TABLE 9

| | | | T. | T. | L. major | | | | |
|---|---|---|---|---|---|---|---|---|---|
| | | | brucei | cruzi | Promastigote | Amastigote | P. falciparum | | |
| | | | EC₅₀ | EC₅₀ | EC5₀ | EC₅₀ | D6 | W2 | C235 |
| Entry | R₁ | R₂ | (μM)^{a} | (μM)^{b} | (μM)^{c} | (μM)^{c} | (EC₅₀)^{d} | (EC₅₀)^{d} | (EC₅₀)^{d} |
| NEU- | CI | OBn | 0.61 | 3.9 | 3.4 | 2.2 | 0.029 | 0.051 | 0.028 |
| 764 | | | | | | | | | |
| NEU- | H | OBn | 0.63 | 9.9 | 1.5 | 1.9 | 0.041 | 0.11 | 0.036 |
| 765 | | | | | | | | | |
| NEU- | CI | OCH₃ | 0.78 | >50 | 5.9 | >15 | 0.033 | 0.21 | 0.058 |
| 766 | | | | | | | | | |
| NEU- | H | H | 1.5 | >50 | 6.1 | >15 | 0.70 | 1.5 | 0.73 |
| 767 | | | | | | | | | |
| NEU- | H | F | 2.0 | >50 | 12 | >15 | 0.36 | 0.78 | 0.38 |
| 768 | | | | | | | | | |
| NEU- | H | CI | 2.4 | 21 | 4.6 | >15 | 0.11 | 0.39 | 0.17 |
| 769 | | | | | | | | | |

^{a}All EC₅₀ values are ±7%.
^{b}All SEM values within 15% unless noted otherwise.
^{c}r² values >0.75.
^{d}All r² values >0.9 unless noted otherwise

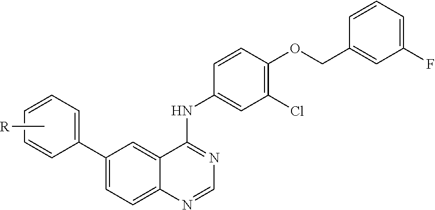

TABLE 10

| | | | T. | T. | | | | | |
|---|---|---|---|---|---|---|---|---|---|
| | | | brucei | cruzi | L. major | | P. falciparum | | |
| | | | EC₅₀ | EC₅₀ | Promatigote | Amastigote | D6 | W2 | C235 |
| Entry | Pos. | R | (μM)^{a} | (μM)^{b} | EC₅₀ (μM)^{d} | EC₅₀ (μM)^{d} | (EC₅₀)^{e} | (EC₅₀)^{e} | (EC₅₀)^{e} |
| NEU- 617 | m | 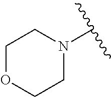 [SMILES: *N1CCOCC1] | 0.042 | 1.8 | 3.0 | 8.0 | 0.23 | 0.68 | 0.37 |
| NEU- 618 | m | 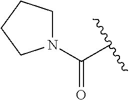 [SMILES: *C(=O)N1CCCC1] | 0.52 | (7)* | >15 | >15 | 0.069 | 0.09 | 0.090 |
| NEU- 634 | p | 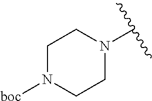 | 6.0 | (8)* | 3.1 | 3.1 | 0.86 | 3.2^{d} | 1.1 |
| NEU- | p | CH₂Ot-Bu | 0.22 | 2.2^{c} | >15 | 4.1 | 0.60 | 2.2 | 0.69 |
| 635 | | | | | | | | | |
| NEU- 636 | p | 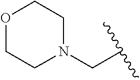 [SMILES: *CN1CCOCC1] | 0.99 | 0.60 | 2.7 | 5.9 | 0.064 | 0.10 | 0.085 |
| NEU- 638 | p | 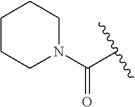 [SMILES: *C(=O)N1CCCCC1] | 0.94 | >50 | 1.0 | 2.9 | 0.25 | 0.88 | 0.36 |
| NEU- 639 | m | 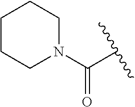 [SMILES: *C(=O)N1CCCCC1] | 0.14 | >50 | 3.5 | 3.5 | 0.52 | 0.96 | 0.57 |
| NEU- | — | H | 3.9 | 1.8 | 4.0 | 22 | 0.79 | 2.6 | 1.2 |
| 706 | | | | | | | | | |
| NEU- 708 | m | 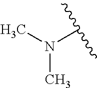 [SMILES: *N(C)C] | 2.2 | (58)* | >15 | >15 | 0.46 | 1.4 | 0.55 |
| NEU- 709 | m | 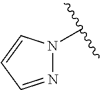 [SMILES: *n1cccn1] | 7.5 | (10)* | >15 | >15 | 19 | 19 | 19 |
| NEU- 710 | p | 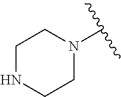 [SMILES: *N1CCNCC1] | 0.37 | 1.4 | >15 | 8.6 | 0.38 | 0.63 | 0.76 |
| NEU- 711 | m | 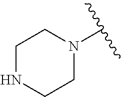 [SMILES: *N1CCNCC1] | 0.25 | 2.2 | — | — | — | — | — |
| NEU- 712 | m | 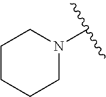 [SMILES: *N1CCCCC1] | 0.76 | (24)* | >15 | >15 | 0.64 | 1.8 | 1.1 |
| NEU- 713 | m | 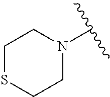 [SMILES: *N1CCSCC1] | 0.77 | (55)* | >15 | 10 | 0.44 | 1.3 | 0.78 |
| NEU- 714 | m | 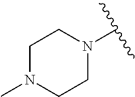 [SMILES: *N1CCN(C)CC1] | 0.55 | (72)* | 1.9 | 4.2 | 0.044 | 0.059 | 0.11 |
| NEU- 733 | p | 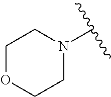 [SMILES: *N1CCOCC1] | 1.9 | (10)* | 4.4 | >15 | 0.27 | 1.1 | 0.29 |
| NEU- 735 | m | 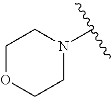 [SMILES: *N1CCOCC1] | 0.23 | 1.3 | 6.6 | >15 | 0.79 | 1.7 | 0.72 |
| NEU- 736 | m | 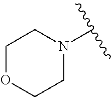 [SMILES: *N1CCOCC1] | 0.18 | (37)* | 2.7 | >15 | 20 | 20 | 20 |
| NEU- 737 | m | 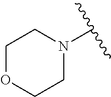 [SMILES: *N1CCOCC1] | 11 | 6.72 | 9.4 | >15 | 3.1 | 3.3 | 4.6 |
| NEU- 782 | m | 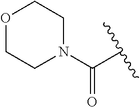 [SMILES: *C(=O)N1CCOCC1] | 0.55 | 3.5 | 3.0 | >15 | 0.76 | 1.1 | 1.2 |
| NEU- 783 | p | 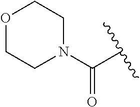 [SMILES: *C(=O)N1CCOCC1] | 1.2 | 2.8 | 0.85 | 1.4 | 0.40 | 0.86 | 0.61 |
| NEU- 784 | p | 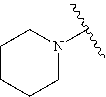 [SMILES: *N1CCCCC1] | 0.38 | 7.2 | >15 | 5.0 | 0.58 | 1.2 | 1.0 |
| NEU- 785 | m | 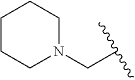 [SMILES: *CN1CCCCC1] | 0.65 | 1.5 | 2.5 | 3.5 | 0.039 | 0.073 | 0.065 |
| NEU- 786 | m | 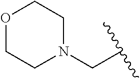 [SMILES: *CN1CCOCC1] | 0.36 | 2.4 | 3.3 | 3.0 | 0.19 | 0.35 | 0.29 |
| NEU- 787 | p | 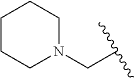 [SMILES: *CN1CCCCC1] | 1.5 | 3.4 | 8.4 | 5.5 | 0.068 | 0.11 | 0.091 |

*% inh at 10 μM.
^{a}All EC₅₀ values are ±7%.
^{b}All SEM values within 35% unless noted otherwise.
^{c}n = 2
^{d}r² values >0.75.
^{e}All r² values >0.9 unless noted otherwise

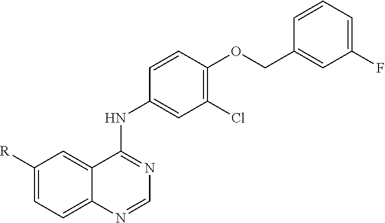

TABLE 11

| | | T. | T. | | | | | |
|---|---|---|---|---|---|---|---|---|
| | | brucei | cruzi | L. major | | P. falciparum | | |
| | | EC₅₀ | % inh | Promatigote | Amastigote | D6 | W2 | C235 |
| Entry | R | (μM)^{a} | at 10 μM | EC₅₀ (μM)^{b} | EC₅₀ (μM)^{b} | (EC₅₀)^{c} | (EC₅₀)^{c} | (EC₅₀)^{c} |
| NEU- 632 | 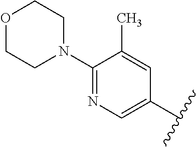 [SMILES: *c1cnc(N2CCOCC2)c(C)c1] | 1.0 | 49 | 3.6 | 1.1 | 0.26 | 0.54 | 0.36 |
| NEU- 637 | 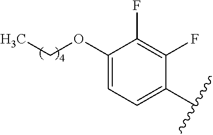 | 6.6 | 52 | >15 | >15 | 17 | 17 | 17 |
| NEU- 640 | 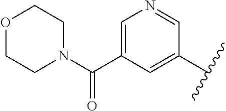 [SMILES: *c1cncc(C(=O)N2CCOCC2)c1] | 0.34 | −2 | >15 | >15 | 0.37 | 0.63 | 0.50 |

^{a}All EC₅₀ values are ±7%.
^{b}r² values >0.75.
^{c}All r² values >0.9 unless noted otherwise

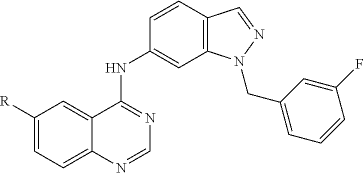

TABLE 12

| | | T. | T. | | | | | |
|---|---|---|---|---|---|---|---|---|
| | | brucei | cruzi | L. major | | P. falciparum | | |
| | | EC₅₀ | EC₅₀ | Promastigote | Amastigote | D6 | W2 | C235 |
| Entry | R | (μM)^{a} | (μM)^{b} | EC₅₀ (μM)^{c} | EC₅₀ (μM)^{c} | (EC₅₀)^{c} | (EC₅₀)^{c} | (EC₅₀)^{c} |
| NEU- 734 | 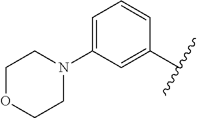 [SMILES: *c1cccc(N2CCOCC2)c1] | 0.84 | (62)* | 0.98 | >15 | 0.91 | 0.74 | 1.1 |
| NEU- 771 | 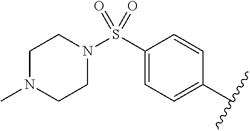 [SMILES: *c1ccc(S(=O)(=O)N2CCN(C)CC2)cc1] | 1.2 | 1.5 | >15 | >15 | 0.039 | 0.090 | 0.040 |

*% inh at 10 μM.
^{a}All EC₅₀ values are ±7%.
^{b}All SEM values within 35% unless noted otherwise.
^{c}All r² values >0.9 unless noted otherwise

Antiparasitic activity of quinoline, isoquinoline, cinnoline, phthalazine, and 3-cyanoquinoline analogs

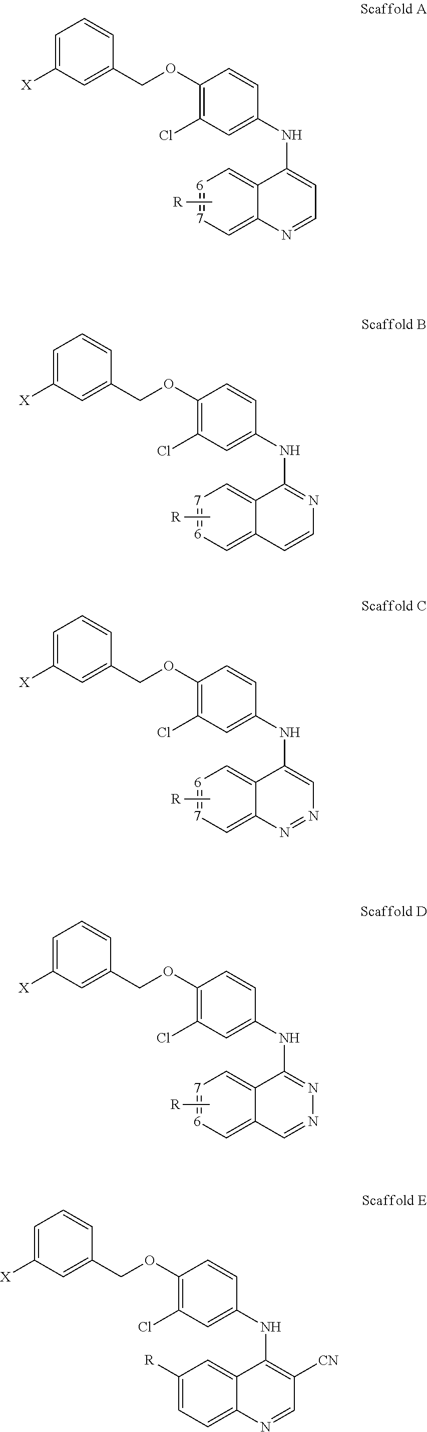

TABLE 13

| | | | | | | Tbb | Tcr | L. major EC₅₀ | | P. fal | HepG2 |
|---|---|---|---|---|---|---|---|---|---|---|---|
| | | | | | | EC₅₀ | EC₅₀ | (μM)^{e} | | EC₅₀ | TC₅₀ |
| NEU | Entry | R | Scaff | Pos | X | (μM) | (μM)^{a} | Promast | Amast | (μM)^{e} | (μM)^{d} |
| NEU-959 NEU-942 | 45 46 | 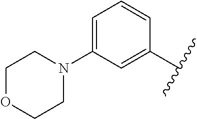 [SMILES: *c1cccc(N2CCOCC2)c1] | A A | 6 7 | F F | 1.0 1.2 | 6.6 2.7 | 0.9 1.4 | 4.0 >3.0 | 0.035 0.061 | 10 >5 |
| NEU-945 NEU-944 | 47 48 | 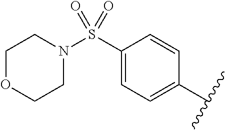 [SMILES: *c1ccc(S(=O)(=O)N2CCOCC2)cc1] | A A | 6 7 | H H | 2.1 0.24 | 49 3.5 | 0.2 ‡ | >3.0 ‡ | 0.032 0.016 | >5 ‡ |
| NEU-960 NEU-961 | 49 50 | 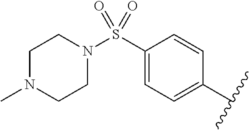 [SMILES: *c1ccc(S(=O)(=O)N2CCN(C)CC2)cc1] | A A | 6 7 | F F | 0.46 0.079 | 5.3 0.73^{b} | 0.6^{f} 1.0^{f} | 2.0 1.6^{f} | 16 0.019 | 6.4 >4 |
| NEU-958 NEU-943 | 51 52 | 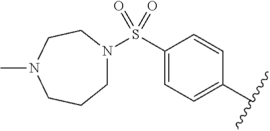 [SMILES: *c1ccc(S(=O)(=O)N2CCCN(C)CC2)cc1] | A A | 6 7 | F F | 0.14 0.087 | 0.93 0.73^{b} | 0.4 0.4 | 2.3 0.88 | 0.086 0.094 | 4.3 5 |
| NEU-947 NEU-946 | 53 54 | 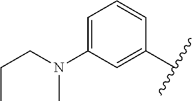 [SMILES: *c1cccc(N(C)CCC)c1] | B B | 7 6 | F F | (0.1)* (12)* | >50 >50 | >15 >15 | 6.0^{f} 2.2^{f} | 2.5 2.8 | >15 >15 |
| NEU-949 NEU-948 | 55 56 | 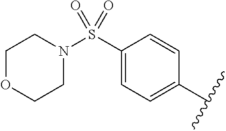 [SMILES: *c1ccc(S(=O)(=O)N2CCOCC2)cc1] | B B | 7 6 | H H | >2.5 0.091 | >50 >50 | >15 >15 | >15 >3 | 0.87 3.4 | >15 >5 |
| NEU-950 NEU-951 | 57 58 | 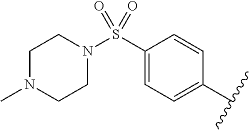 [SMILES: *c1ccc(S(=O)(=O)N2CCN(C)CC2)cc1] | B B | 7 6 | F F | 1.8 0.10 | >50 >50 | >15 2.7 | >15 >15 | 0.04 0.11 | >15 >15 |
| NEU-962 NEU-963 | 59 60 | 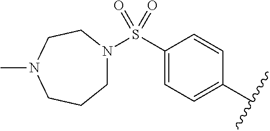 [SMILES: *c1ccc(S(=O)(=O)N2CCCN(C)CC2)cc1] | B B | 7 6 | F F | 0.73 0.39 | 4.7^{c} 33 | 1.3 0.44 | 2.2 4.4^{f} | 0.086 0.41 | >15 >15 |
| NEU-1012 NEU-1014 | 61 62 | 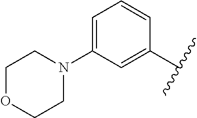 [SMILES: *c1cccc(N2CCOCC2)c1] | C C | 6 7 | F F | 1.1 0.89 | >50 45 | >15 9.8 | >15 >15 | 0.54 0.20 | >15 11 |
| NEU-1002 NEU-1015 | 63 64 | 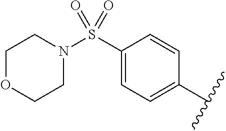 [SMILES: *c1ccc(S(=O)(=O)N2CCOCC2)cc1] | C C | 6 7 | H H | 1.2 1.2 | >50 15 | >15 >15 | >15 >15 | 0.82 0.15 | >15 >11 |
| NEU-1003 NEU-1016 | 65 66 | 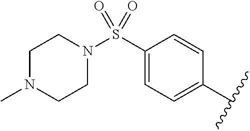 [SMILES: *c1ccc(S(=O)(=O)N2CCN(C)CC2)cc1] | C C | 6 7 | F F | 0.98 1.0 | >50 3.1 | >15 5.7 | 1.9 2.0 | 0.13 0.014 | >15 >15 |
| NEU-1013 NEU-1017 | 67 68 | 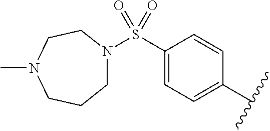 [SMILES: *c1ccc(S(=O)(=O)N2CCCN(C)CC2)cc1] | C C | 6 7 | F F | 0.58 0.21 | 2.2 49 | 4.1 6.4 | 1.06 0.24 | 0.027 0.003 | 7.3 15 |
| NEU-1035 NEU-1037 | 69 70 | 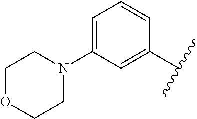 [SMILES: *c1cccc(N2CCOCC2)c1] | D D | 7 6 | F F | 0.29 1.4 | >50 >50 | >15 >15 | 1.7^{f} 2.2 | 0.11 0.24 | >15 >15 |
| NEU-1036 NEU-1039 | 71 72 | 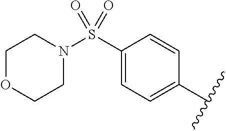 [SMILES: *c1ccc(S(=O)(=O)N2CCOCC2)cc1] | D D | 7 6 | H H | 0.86 0.99 | >50 >50 | >3.4 >15 | >4.0 >15 | 0.54 0.11 | >5.2 >15 |
| NEU-1043 NEU-1041 | 73 74 | 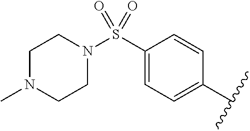 [SMILES: *c1ccc(S(=O)(=O)N2CCN(C)CC2)cc1] | D D | 7 6 | F F | 2.0 1.2 | >50^{d} 18^{d} | >3.2 1.3^{f} | >4.0 >15 | 0.13 0.045 | >5 >15 |
| NEU-1044 NEU-1042 | 75 76 | 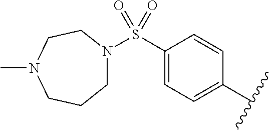 [SMILES: *c1ccc(S(=O)(=O)N2CCCN(C)CC2)cc1] | D D | 7 6 | F F | 0.62 0.51 | 17^{d} 1.3^{d} | >15 0.41 | 5.6^{f} 1.1 | 0.094 0.029 | 14 3.4 |
| NEU-925 NEU-996 | 77 78 | 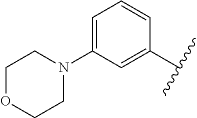 [SMILES: *c1cccc(N2CCOCC2)c1] | E E | — — | F H | (49)* 0.76 | >50 >50 | >15 >15 | >15 >15 | 0.48 0.97 | >15 >15 |
| NEU-926 NEU-914 | 79 80 | 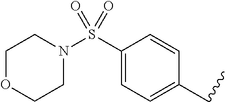 | E E | — — | F H | (15)* (33)* | >50 >50 | >15 >15 | >15 >15 | 0.16 0.28 | >15 >15 |
| NEU-993 NEU-994 | 81 82 | 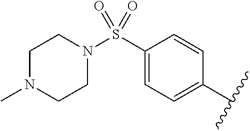 [SMILES: *c1ccc(S(=O)(=O)N2CCN(C)CC2)cc1] | E E | — — | F H | 1.1 1.9 | >50 >50 | >15 4.1 | >15 >15 | 0.082 0.21 | >15 >15 |
| NEU-924 NEU-995 | 83 84 | 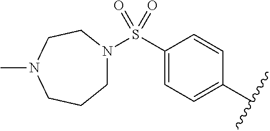 [SMILES: *c1ccc(S(=O)(=O)N2CCCN(C)CC2)cc1] | E E | — — | F H | 0.35 0.43 | 0.09^{b} 0.95 | 0.92 1.6 | 1.6 2.3^{f} | 0.047 0.058 | >15 13 |

‡Insoluble under assay conditions
*% inh at 5 μM
^{a}All SEM values within 25% unless noted otherwise.
^{b}SEM values within 40%.
^{c}SEM = 0.89.
^{d}n = 1
^{e}All r² values >0.9 unless noted otherwise.
^{f}r² values >0.75

Five (5) representative compounds were selected from the most potent to be tested for their physicochemical properties (Table 14). All compounds tested were >99% plasma protein bound with thermodynamic aqueous solubility <1 μM. Such properties are undoubtedly a result of the high molecular weights and clogP values; these issues remain a goal of ongoing efforts.

TABLE 14

| | | | | | | | Human | Male rat |
|---|---|---|---|---|---|---|---|---|
| | | | | | | | liver | hepatocytes |
| | | | | | | | microsomes | median |
| | | | | | Protein | | median | CLint |
| | | Molecular | | | binding | Solubility | CLint | (μl/min/1E6 |
| NEU | Entry | weight | clogP | logD | (% free) | (uM) | (μl/min/mg) | cells) |
| NEU-617 | 1 | 541 | 7.31 | 3.2 | <1 | <1 | 63.03 | 44.5 |
| NEU-945 | 47 | 586 | 6.22 | 3.5 | <1 | <1 | 78.87 | 13.93 |
| NEU-961 | 50 | 617 | 6.43 | NV¹ | <1 | NA³ | 142.7 | 20.24 |
| NEU-1017 | 68 | 632 | 5.51 | NV² | <1 | <1 | 99.32 | 36.72 |
| NEU-924 | 83 | 656 | 6.35 | NV² | <1 | NA³ | 151.8 | 37.95 |

¹Poor chromatography
²No signal observed in buffer layer
³Peak in standard samples only

### Experimental Section

Chemical Synthesis. Unless otherwise noted, reagents were obtained from Sigma-Aldrich, Inc. (St. Louis, Mo.) or Frontier Scientific Services, Inc. (Newark, Del.) and used as received. Boronic acids and aniline reagents were purchased unless the synthesis is specifically described below. Reaction solvents were purified by passage through alumina columns on a purification system manufactured by Innovative Technology (Newburyport, Mass.). NMR spectra were obtained with Varian NMR systems operating at 400 or 500 MHz for 1H acquisitions as noted. LCMS analysis was performed using a Waters Alliance reverse-phase HPLC, with single-wavelength UV-visible detector and LCT Premier time-of-flight mass spectrometer (electrospray ionization). All newly synthesized compounds that were submitted for biological testing were deemed >95% pure by LCMS analysis (UV and ESI-MS detection) prior to submission for biological testing. Preparative LCMS was performed on a Waters FractionLynx system with a Waters MicroMass ZQ mass spectrometer (electrospray ionization) and a single-wavelength UV-visible detector, using acetonitrile/water gradients with 0.1% formic acid. Fractions were collected on the basis of triggering using UV and mass detection.

Yields reported for products obtained by preparative HPLC represent the amount of pure material isolated; impure fractions were not repurified. Screening data of selected boronates has been made freely available as a shared data set at www.collaborativedrugdiscovery.com.

4-Chloro-6-iodoquinazoline Hydrochloride (13). Yield: 85%.1H NMR (500 MHz, DMSO-d6) δ: 8.39 (d, J=1.95 Hz, 1H), 8.29 (s, 1H), 8.13 (dd, J=1.95, 8.30 Hz, 1H), 7.49 (d, J=8.30 Hz, 1H). MS: m/z=290.83 (M+H)+.

N-(3 -Chloro-443 -fluorobenzyl)oxy)phenyl)-6-iodoquinazolin-4-amine Hydrochloride (14).46 Yield: 84%. 1H NMR (500 MHz, DMSO-d6) δ: 11.21 (br s, 1H), 9.16 (s, 1H), 8.92 (s, 1H), 8.34 (d, J=8.79 Hz, 1H), 7.93 (d, J=2.44 Hz, 1H), 7.64-7.68 (m, 2H), 7.46-7.51 (m, 1H), 7.30-7.37 (m, 2H), 7.20 (dt, J=2.44, 8.79 Hz, 1H), 5.30 (s, 2H). MS: m/z=505.85 (M+H)+.

Libraries of 10 were synthesized by Suzuki coupling of 14 with respective boronic acid/esters following general procedure A. Into glass vials was combined N-(3-chloro-4-((3-fluorobenzyl)oxy)phenyl)-6-iodoquinazolin-4-amine (14, 100 μM), boronic acids/esters (120 μmol), and tetrakis(triphenylphosphine)palladium(0) (7 μmol). To the reaction mixture was added 1,2-dimethoxyethane (2 mL), ethanol (1.33 mL), and a 2 M aqueous solution of sodium carbonate (0.301 mL, 600 μM). The vials were capped and shaken at 80° C. for 18 h. The progress of the reaction was followed by LC-MS. Reaction mixture was evaporated to dryness. Crude products were purified using flash column chromatography or by dissolving in DMSO and purifying by reverse phase HPLC using a gradient of 30-100% acetonitrile in water containing 0.1% formic acid.

N-(3-Chloro-4-((3-fluorobenzyl)oxy)phenyl)-6-(4-(morpholinosulfonyl)phenyl)quinazolin-4-amine (10a). Yield: 48.1%. 1H NMR (500 MHz, DMSO-d6) δ: 10.00 (s, 1H), 8.92 (d, J=1.46 Hz, 1H), 8.64 (s, 1H), 8.28 (dd, J=1.95, 8.79 Hz, 1H), 8.17 (d, J=8.79 Hz, 2H), 8.04 (d, J=2.44 Hz, 1H), 7.92 (d, J=8.79 Hz, 3H), 7.76 (dd, J=2.45, 8.80 Hz, 1H), 7.46-7.50 (m, 1H), 7.30-7.36 (m, 3H), 7.19-7.23 (m, 1H), 5.28 (s, 2H), 3.66-3.68 (m, 4H), 2.93-2.95 (m, 4H). MS: m/z=605.2 (M+H)+.

N-(3-Chloro-4-((3-fluorobenzyl)oxy)phenyl)-6-(4-methylnaphthalen-1-yl)quinazolin-4-amine (10b). Yielded 1 mg (2.6%) as a yellow film. 1H NMR (400 MHz, DMSO-d6) δ: 9.82 (s, 1H), 8.65 (s, 2H), 8.15 (d, J=8.8 Hz, 1H), 8.03 (d, J=2.2 Hz, 1H), 7.87-7.96 (m, 2H), 7.81 (d, J=8.8 Hz, 1H), 7.71-7.76 (m, 1H), 7.63 (t, J=8.0 Hz, 1H), 7.49-7.57 (m, 2H), 7.42-7.49 (m, 2H), 7.31 (t, J=6.0 Hz, 2H), 7.25 (d, J=8.8 Hz, 1H), 7.17 (t, J=7.3 Hz, 1H), 5.24 (s, 2H), 2.74 (s, 3H). MS: m/z=520.1 (M+H)+.

4-(4-4-((3-Chloro-4-((3-fluorobenzyl)oxy)phenyl)amino)-quinazolin-6-yl)-N-ethyl-2-fluorobenzamide (10c). Yielded 1 mg (2.4%) as a yellow film. 1H NMR (400 MHz, DMSO-d6) δ: 9.98 (s, 1H), 8.87 (s, 1H), 8.58-8.61 (s, 1H), 8.35-8.42 (m, 1H), 8.26-8.34 (m, 1H), 8.01 (s, 1H), 7.87 (d, J=4.4 Hz, 1H), 7.84 (m, 2H), 7.79 (d, J=8.1 Hz, 1H), 7.70-7.76 (m, 1H), 7.47 (q, J=7.3 Hz, 1H), 7.32 (dd, JA=13.2 Hz, JB=7.3 Hz, 3H), 7.18 (t, J=8.8 Hz, 1H), 5.27 (s, 2H), 3.29 (q, J=8.0 Hz, 2H), 1.14 (t, J=7.0 Hz, 3H). MS: m/z=545.2 (M+H)+.

N-(3-Chloro-4-((3-fluorobenzyl)oxy)phenyl)-6-(3-(5-methyl-1,3,4-oxadiazol-2-1)phenyl)quinazolin-4-amine (10d). Obtained 1 mg (2.5% yield) as a yellow oil. 1H NMR (400 MHz, DMSO-d6) δ: 9.09-10.04 (brs, 1H), 8.88 (s, 1H), 8.61 (s, 1H), 8.42 (s, 1H), 8.37 (s, 1H), 8.26 (d, J=8.8 Hz, 1H), 8.11 (d, J=8.1 Hz, 1H), 8.0 (m, 2H), 7.89 (d, J=8.8 Hz, 1H), 7.72-7.81 (m, 2H), 7.43-7.51 (m, 1H), 7.28-7.36 (m, 2H), 7.14-7.22 (m, 1H) 5.26 (s, 2H), 2.65 (s, 3H). MS: m/z=538.1 (M+H)+.

3-(4-((3-Chloro-4-((3-fluorobenzyl)oxy)phenyl)amino)-quinazolin-6-yl)-N-cyclopropylbenzamide (10e). Yielded 0.8 mg (2.0%) as a yellow film. 1H NMR (400 MHz, DMSO-d6) δ: 10.00 (s, 1H), 8.83 (s, 1H), 8.63 (d, J=3.7 Hz, 1H), 8.59 (s, 1H), 8.35 (s, 1H), 8.26 (d, J=5.1 Hz, 1H), 8.22 (s, 1H), 7.97-8.05 (m, 1H), 7.87 (t, J=8.1 Hz, 2H), 7.74 (dd, JA=8.8 Hz, JB=2.0 Hz, 1H), 7.63 (t, J=7.7 Hz, 1H), 7.43-7.52 (m, 1H), 7.25-7.35 (m, 2H), 7.17 (t, J=8.0 Hz, 1H), 5.27 (s, 2H), 2.85-2.92 (m, 1H), 0.69-0.76 (m, 2H), 0.58-0.65 (m, 2H). MS: m/z=539.2 (M+H)+.

N-(3-Chloro-4-((3-fluorobenzyl)oxy)phenyl)-6-(quinolin-5-yl)quinazolin-4-amine (10f). Yielded 3.2 mg (8.4%) as a yellow solid. 1H NMR (400 MHz, DMSO-d6) δ: 9.81 (s, 1H), 8.98 (d, J=2.9 Hz, 1H), 8.70 (s, 1H), 8.68 (s, 1H), 8.21-8.27 (m, 1H), 8.14 (d, J=8.1 Hz, 1H), 8.03 (d, J=2.2 Hz, 1H), 7.95-8.00 (m, 1H), 7.88-7.95 (m, 2H),7.68-7.96 (m, 2H), 7.53-7.59 (dd, JA=8.4 Hz, JB=4.0 Hz, 1H), 7.46 (q, J=8.0 Hz, 1H), 7.22-7.33 (m, 3H), 7.17 (t, J=8.8 Hz, 1H), 5.23 (s, 2H). MS: m/z=507.2 (M+H)+.

N-(3-Chloro-4-((3-fluorobenzyl)oxy)phenyl)-6-(2-phenoxyphenyl)quinazolin-4-amine (10g). Yielded 1.4 mg (2.4%) as an orange oil. 1H NMR (400 MHz, DMSO-d6) δ: 9.85 (s, 1H), 8.65 (s, 1H), 8.58 (s, 1H), 8.01-8.06 (m, 2H), 7.72-7.78 (m, 2H), 7.67 (d, J=6.6 Hz, 1H), 7.42-7.51 (m, 2H), 7.24-7.39 (m, 6H), 7.19 (t, J=7.3 Hz, 1H), 7.00-7.09 (m, 2H), 6.94 (d, J=8.1 Hz, 2H), 5.27 (s, 2H). MS: m/z=548.1 (M+H)+.

6-(Benzo[b]thiopen-2-yl)-N-(3-chloro-4-((3-fluorobenzyl)-oxy)phenyl)quinazolin-4-amine (10h). Obtained 5.4 mg (14% yield) as a yellow solid. 1H NMR (400 MHz, DMSO-d6) δ: 8.75 (s, 1H), 8.15 (t, J=8.8 Hz, 1H), 8.03 (m, 1H), 7.96 (d, J=8.8 Hz, 1H), 7.87 (d, J=2.1 Hz, 2H), 7.82 (d, J=7.3 Hz, 1H), 7.68 (s, 1H), 7.51-7.59 (m, 1H), 7.49 (s, 1H), 7.32-7.44 (m, 3H), 7.19-7.25 (m, 1H), 6.91-7.09 (m, 2H), 5.27 (s, 2H). MS: m/z=512.0 (M+H)+.

4-(4-((3-Chloro-4-((3-fluorobenzyl)oxy)phenyl)amino)-quinazolin-6-yl)phenol (10i). Yielded 1 mg (2.4%) as a yellow film. 1H NMR (400 MHz, DMSO-d6) δ: 10.09 (s, 1H), 8.98 (s, 1H), 8.63 (s, 2H), 8.45 (s, 1H), 8.42 (s, 1H), 8.05 (s, 1H), 7.96 (d, J=8.8 Hz, 1H), 7.82 (d, J=9.5 Hz, 1H), 7.43-7.51 (m, 2H), 7.27-7.36 (m, 2H), 7.14-7.22 (m, 1H), 6.66-6.72 (m, 2H), 5.27 (s, 2H). MS: m/z=472.1 (M+H)+.

5-(4-4-((3-Chloro-44(3-fluorobenzyl)oxy)phenyl)amino)-quinazolin-6-yl)pyrimidine-2,4(1H,3H)-dione (10j). Yielded 2.6 mg (7.1%) as a yellow solid. 1H NMR (400 MHz, DMSO-d6) δ: 9.76-9.82 (brs, 1H), 8.47-8.59 (m, 2H), 8.30 (s, 1H), 8.04 (m, 2H), 7.81-7.88 (m, 1H), 7.71-7.80 (m. 2H), 7.59-7.67 (m, 1H), 7.43-7.50 (m, 1H), 7.31-7.36 (m, 1H), (m, 7.23-7.31 (m, 2H), 7.18 (t, J=8.4 Hz, 1H), 5.26 (s, 2H). MS: m/z=490.0 (M+H)+.

N-(3-Chloro-4-((3-fluorobenzyl)oxy)phenyl)-6-(3-(morpholinosulfonyl)phenyl)quinazolin-4-amine (10k). Yield: 30.4%. 1H NMR (500 MHz, DMSO-d6) δ: 10.01 (s, 1H), 8.85 (d, J=1.95 Hz, 1H), 8.63 (s, 1H), 8.24-8.27 (m, 2H), 8.12 (t, J=1.71 Hz, 1H), 8.03 (d, J=2.93 Hz, 1H), 7.86-7.92 (m, 2H), 7.80-7.85 (m, 1H), 7.73 (dd, J=2.44, 8.79 Hz, 1H), 7.46-7.52 (m, 1H), 7.30-7.35 (m, 3H), 7.17-7.22 (m, 1H), 5.28 (s, 2H), 3.66 (t, J=4.90 Hz, 4H), 2.95 (t, J=4.40 Hz, 4H). MS: m/z=605.1 (M+H)+.

N-(3-Chloro-4-((3-fluorobenzyl)oxy)phenyl)-6-(4-(piperidin-1-ylsulfonyl)phenyl)quinazolin-4- amine (101). Yield: 14.6%. 1H NMR (500 MHz, DMSO-d6) δ: 10.00 (s, 1H), 8.91 (d, J=1.46 Hz, 1H), 8.64 (s, 1H), 8.27 (dd, J=1.95, 8.30 Hz, 1H), 8.14 (d, J=8.30 Hz, 2H), 8.04 (d, J=2.93 Hz, 1H), 7.89-7.92 (m, 3H), 7.76 (dd, J=2.69, 9.03 Hz, 1H), 7.46-7.51 (m, 1H), 7.31-7.36 (m, 3H), 7.20 (dt, J=2.44, 8.55 Hz, 1H), 5.28 (s, 2H), 2.95-2.98 (m, 4H), 1.55-1.60 (m, 4H), 1.39-1.40 (m, 2H). MS: m/z=603.2 (M+H)+.

N-(3-Chloro-4-((3-fluorobenzyl)oxy)phenyl)-6-(3-(piperidin-1-ylsulfonyl)phenyl)quinazolin-4-amine (10m). Yield: 24.8%. 1H NMR (500 MHz, DMSO-d6) δ: 10.01 (s, 1H), 8.84 (d, J=1.95 Hz, 1H), 8.62 (s, 1H), 8.24 (dd, J=1.95, 8.79 Hz, 1H), 8.21 (d, J=7.35 Hz, 1H), 8.11 (t, J=1.71 Hz, 1H), 8.02 (d, J=2.44 Hz, 1H), 7.90 (d, J=8.30 Hz, 1H), 7.82-7.86 (m, 1H), 7.79-7.81 (m, 1H), 7.72 (dd, J=2.69, 9.03 Hz, 1H), 7.45-7.51 (m, 1H), 7.29-7.36 (m, 3H), 7.19 (dt, J=2.44, 8.55 Hz, 1H), 5.27 (s, 2H), 2.96 (t, J=5.4 Hz, 4Hm, 4H), 1.52-1.60 (m, 4H), 1.33-1.40 (m, 2H). MS: m/z=603.2 (M+H)+.

N-(3-Chloro-4-((3-fluorobenzypoxy)phenyl)-6-(4-(pyrrolidin-1-ylsulfonyl)phenyl)quinazolin-4-amine (10n). Yield: 19.6%. 1H NMR (500 MHz, DMSO-d6) δ: 10.00 (s, 1H), 8.91 (d, J=1.95 Hz, 1H), 8.63 (s, 1H), 8.27 (dd, J=1.95, 8.79 Hz, 1H), 8.13 (d, J=8.30 Hz, 2H), 8.03 (d, J=2.44 Hz, 1H), 7.98 (d, J=8.79 Hz, 2H), 7.90 (d, J=8.79 Hz, 1H), 7.76 (dd, J=2.44, 8.79 Hz, 1H), 7.45-7.52 (m, 1H), 7.29-7.36 (m, 3H), 7.20 (dt, J=2.44, 8.55 Hz, 1H), 5.28 (s, 2H), 3.22 (t, J=6.84 Hz, 4H), 1.66-1.72 (m, 4H). MS: m/z=589.1 (M+H)+.

N-(3-Chloro-4-((3-fluorobenzypoxy)phenyl)-6-(3-(pyrrolidin-1-ylsulfonyl)phenyl)quinazolin-4-amine (10o). Yield: 28.2%. 1H NMR (500 MHz, DMSO-d6) δ: 10.03 (s, 1H), 8.85 (d, J=1.46 Hz, 1H), 8.62 (s, 1H), 8.26 (dd, J=1.95, 8.79 Hz, 1H), 8.18-8.23 (m, 2H), 8.03 (d, J=2.44 Hz, 1H), 7.88-7.93 (m, 2H), 7.81-7.87 (m, 1H), 7.73 (dd, J=2.44, 8.79 Hz, 1H), 7.45-7.52 (m, 1H), 7.30-7.37 (m, 3H), 7.20 (dt, J=2.44, 8.55 Hz, 1H), 5.28 (s, 2H), 3.23 (t, J=6.84 Hz, 4H), 1.68 (td, J=3.54, 6.59 Hz, 4H). MS: m/z=589.2 (M+H)+.

N-(3-Chloro-4-((3-fluorobenzyl)oxy)phenyl)-6-(4-((4-methylpiperazin-1-yl)sulfonyl)phenyl)quinazolin-4-amine (10p). Yield: 30.6%. 1H NMR (500 MHz, DMSO-d6) δ: 9.95 (s, 1H), 8.87 (d, J=1.46 Hz, 1H), 8.59 (s, 1H), 8.23 (dd, J=1.95, 8.79 Hz, 1H), 8.11 (d, J=8.30 Hz, 2H), 7.99 (d, J=2.44 Hz, 1H), 7.83-7.90 (m, 3H), 7.71 (dd, J=2.69, 9.03 Hz, 1H), 7.40-7.47 (m, 1H), 7.25-7.32 (m, 3H), 7.15 (dt, J=2.44, 8.55 Hz, 1H), 5.23 (s, 2H), 2.91 (br s, 4H), 2.34 (br s, 4H), 2.11 (s, 3H). MS: m/z=618.2 (M+H)+.

N-(3-Chloro-4-((3-fluorobenzyl)oxy)phenyl)-6-(4((4-methyl-1,4-diazepan-1-yl)sulfonyl)phenyl)quinazolin-4-amine (10q). Yield: 42.8%. 1H NMR (500 MHz, DMSO-d6) δ: 10.00 (s, 1H), 8.91 (d, J=1.95 Hz, 1H), 8.63 (s, 1H), 8.27 (dd, J=1.95, 8.79 Hz, 1H), 8.12 (d, J=8.80 Hz, 2H), 8.03 (d, J=2.44 Hz, 1H), 7.96 (d, J=8.30 Hz, 2H), 7.90 (d, J=8.79 Hz, 1H), 7.76 (dd, J=2.69, 9.03 Hz, 1H), 7.45-7.50 (m, 1H), 7.30-7.36 (m, 3H), 7.20 (dt, J=2.44, 8.55 Hz, 1H), 5.28 (s, 2H), 3.37-3.39 (m, 2H), 3.34 (t, J=6.10 Hz, 2H), 2.61-2.64 (m, 2H), 2.54-2.58 (m, 2H), 2.28 (s, 3H), 1.74-1.80 (m, 2H). MS: m/z=632.2 (M+H)+.

N-(3-Chloro-4-((3-fluorobenzyl)oxy)phenyl)-6- (4-(thiomorpholinosulfonyl)phenyl)quinazolin-4-amine (10r). Yield: 5.6%. 1H NMR (500 MHz, DMSO-d6) δ: 10.00 (s, 1H), 8.92 (d, J=1.47 Hz, 1H), 8.64 (s, 1H), 8.28 (dd, J=1.95, 8.79 Hz, 1H), 8.16 (d, J=8.79 Hz, 2H), 8.04 (d, J=2.44 Hz, 1H), 7.89-7.95 (m, 3H), 7.76 (dd, J=2.45, 8.80 Hz, 1H), 7.46-7.52 (m, 1H), 7.30-7.37 (m, 3H), 7.20 (dt, J=2.20, 8.67 Hz, 1H), 5.28 (s, 2H), 3.28 (t, J=4.35 Hz, 4H), 2.71 (t, J=5.35 Hz, 4H). MS: m/z=621.2 (M+H)+.

N-(3-Chloro-4-((3-fluorobenzypoxy)phenyl)-6-(4-(piperazin-1-ylsulfonyl)phenyl)quinazolin-4-amine (10s). To glass vials was weighed 46 mg of 14 (0.085 mmoL) and tert-butyl 4-((4-(4,4,5,5- tetramethyl-1,3,2-dioxaborolan-2-yl)phenyl)sulfonyl)piperazine-1-carboxylate (38.4 mg 0.085 mmol) and tetrakis(triphenylphosphine)- palladium(0) (0.006 mmol). To the reaction mixture was added 1,2-dimethoxyethane (0.4 mL), ethanol (0.3 mL), and a 2 M aqueous solution of sodium carbonate (0.255 mL, 0.51 mmol). The vials were capped and shaken at 85° C. for 12 h, and progress of the reaction was monitored by LC-MS. The reaction mixture was evaporated to dryness, and the residue was dissolved in DMSO and purified by reverse phase HPLC using a gradient of 5-100% acetonitrile in water containing 0.1% formic acid, providing the Boc-protected compound in 23.7% yield. 1H NMR (500 MHz, DMSO-d6) δ: 9.99 (s, 1H), 8.91 (d, J=1.95 Hz, 1H), 8.64 (s, 1H), 8.28 (dd, J=1.95, 8.79 Hz, 1H), 8.16 (d, J=8.79 Hz, 2H), 8.04 (d, J=2.44 Hz, 1H), 7.89-7.93 (m, 3H), 7.76 (dd, J=2.44, 8.79 Hz, 1H), 7.46-7.52 (m, 1H), 7.30-7.36 (m, 3H), 7.20 (dt, J=2.44, 8.79 Hz, 1H), 5.28 (s, 2H), 3.41-3.46 (m, 4H), 2.94 (t, J=4.64 Hz, 4H), 1.34 (s, 9H). MS: m/z=704.3 (M+H)+. To a solution of this compound (0.015 mmol) in 0.4 mL of dichloromethane was added trifluoroacetic acid (200 μmol, 0.154 mL). The reaction mixture was stirred for 12 h at 25° C. Volatiles were removed in vacuo, and the crude product was triturated with hexanes to afford a crude solid that was purified via flash column chromatography (0-10% MeOH-DCM) to afford the desired compound 10t. Yield: 78%. 1H NMR (500 MHz, DMSO-d6) δ: 9.01 (s, 1H), 8.80 (br s, 1H), 8.59 (br s, 2H), 8.39 (d, J=7.81 Hz, 1H), 8.22 (d, J=8.30 Hz, 2H), 7.92-8.03 (m, 4H), 7.72 (dd, J=2.44, 8.79 Hz, 1H), 7.47-7.52 (m, 1H), 7.32-7.37 (m, 3H), 7.21 (dt, J=2.20, 8.67 Hz, 1H), 5.31 (s, 2H), 3.25 (br s, 4H), 3.18 (br s, 4H). MS: m/z=604.2 (M+H)+.

N-(3-Chloro-4-((3-fluorobenzyl)oxy)phenyl)-6-(4-(methylsulfonyl)phenyl)quinazolin-4-amine (10t). Yield: 31.8%. 1H NMR (500 MHz, DMSO-d6) δ: 10.00 (s, 1H), 8.92 (d, J=1.95 Hz, 1H), 8.64 (s, 1H), 8.29 (dd, J=1.95, 8.79 Hz, 1H), 8.14 (d, J=8.80 Hz, 2H), 8.11 (d, J=8.30 Hz, 2H), 8.03 (d, J=2.44 Hz, 1H), 7.92 (d, J=8.79 Hz, 1H), 7.76 (dd, J=2.44, 8.79 Hz, 1H), 7.45-7.53 (m, 1H), 7.31-7.37 (m, 3H), 7.20 (dt, J=2.20, 8.42 Hz, 1H), 5.28 (s, 2H), 3.31 (s, 3H). MS: m/z=534.1 (M+H)+.

N-(3-Chloro-4-((3-fluorobenzyl)oxy)phenyl)-6-(3- (methylsulfonyl)phenyl)quinazolin-4-amine (10u). Yield: 34.9%. 1H NMR (500 MHz, DMSO-d6) δ: 10.02 (s, 1H), 8.88 (s, 1H), 8.63 (s, 1H), 8.38 (s, 1H), 8.30 (dd, J=1.46, 8.79 Hz, 1H), 8.25 (d, J=8.30 Hz, 1H), 7.99-8.06 (m, 2H), 7.92 (d, J=8.79 Hz, 1H), 7.84-7.87 (m, 1H), 7.74 (dd, J=2.44, 8.79 Hz, 1H), 7.45-7.53 (m, 1H), 7.30-7.36 (m, 3H), 7.20 (dt, J=1.71, 8.67 Hz, 1H), 5.28 (s, 2H), 3.35 (s, 3H). MS: m/z=534.2 (M+H)+.

2-(4-((3-Chloro-4-((3-fluorobenzyl)oxy)phenyl)amino)-quinazolin-6-yl)-N,N-dimethylbenzenesulfonamide (10v). Yield: 35%. 1H NMR (500 MHz, DMSO-d6) δ: 9.78 (s, 1H), 8.65 (s, 1H), 8.52 (d, J=0.98 Hz, 1H), 8.06 (d, J=2.44 Hz, 1H), 8.01 (dd, J=0.98, 7.81 Hz, 1H), 7.83 (dt, J=1.46, 9.03 Hz, 1H), 7.76-7.81 (m, 3H), 7.72 (dt, J=1.22, 7.69 Hz, 1H), 7.54 (dd, J=0.98, 7.81 Hz, 1H), 7.45-7.50 (m, 1H), 7.30-7.34 (m, 2H), 7.27 (d, J=8.79 Hz, 1H), 7.19 (dt, J=2.44, 8.55 Hz, 1H), 5.26 (s, 2H), 2.45 (s, 6H). MS: m/z=563.2 (M+H)+.

N-(tert-Butyl)-2-(4-((3-chloro-4-((3-fluorobenzyl)oxy)-phenyl)amino)quinazolin-6-yl)benzenesulfonamide (10w). Yield: 12.5%. 1H NMR (500 MHz, DMSO-d6) δ: 9.79 (s, 1H), 8.64 (s, 1H), 8.53 (d, J=1.95 Hz, 1H), 8.11 (dd, J=1.22, 8.06 Hz, 1H), 8.05 (d, J=2.44 Hz, 1H), 7.86 (dd, J=1.71, 8.55 Hz, 1H), 7.70-7.79 (m, 3H), 7.63-7.67 (m, 1H), 7.45-7.50 (m, 2H), 7.30-7.35 (m, 2H), 7.27 (d, J=9.28 Hz, 1H), 7.19 (dt, J=2.44, 8.55 Hz, 1H), 6.90 (s, 1H), 5.26 (s, 2H), 1.04 (s, 9H). MS: m/z=591.2(M+H)+.

N-(3-Chloro-4-((3-fluorobenzyl)oxy)phenyl)-6- (3-morpholinophenyl)quinazolin-4-amine (23a). Yield: 38%. 1H NMR (500 MHz, DMSO-d6) δ: 9.90 (s, 1H), 8.76 (d, J=1.95 Hz, 1H), 8.60 (s, 1H), 8.18 (dd, J=1.71, 8.55 Hz, 1H), 8.04 (d, J=2.93 Hz, 1H), 7.84 (d, J=8.79 Hz, 1H), 7.76 (dd, J=2.44, 8.79 Hz, 1H), 7.45-7.51 (m, 1H), 7.39-7.42 (m, 1H), 7.28-7.35 (m, 5H), 7.19 (dt, J=2.44, 8.55 Hz, 1H), 7.03 (dd, J=1.95, 8.30 Hz, 1H), 5.27 (s, 2H), 3.79 (t, J=4.9 Hz, 4H), 3.24 (t, J=4.86 Hz, 4H). MS: m/z=541.2 (M+H)+.

N-(3-Chloro-4-((3-fluorobenzyl)oxy)phenyl)-6-(4-morpholinophenyl)quinazolin-4-amine (23b). Yield: 35.2%. 1H NMR (500 MHz, DMSO-d6) δ: 9.87 (s, 1H), 8.72 (d, J=1.95 Hz, 1H), 8.55 (s, 1H), 8.16 (dd, J=1.95, 8.79 Hz, 1H), 8.03 (d, J=2.93 Hz, 1H), 7.78-7.82 (m, 3H), 7.76 (dd, J=2.44, 8.79 Hz, 1H), 7.45-7.51 (m, 1H), 7.28-7.35 (m, 3H), 7.19 (dt, J=2.69, 8.67 Hz, 1H), 7.11 (d, J=8.79 Hz, 2H), 5.27 (s, 2H), 3.76-3.80 (m, 4H), 3.19-3.23 (m, 4H). MS: m/z=541.04 (M+H)+.

N-(3-Chloro-4-((3-fluorobenzypoxy)phenyl)-6-(3-(morpholinomethyl)phenyl)quinazolin-4-amine (23c). Yield: 54.5%. 1H NMR (500 MHz, DMSO-d6) δ: 9.92 (s, 1H), 8.77 (s, 1H), 8.59 (s, 1H), 8.15 (d, J=8.79 Hz, 1H), 8.03 (d, J=1.95 Hz, 1H), 7.85 (d, J=8.79 Hz, 1H), 7.72-7.80 (m, 3H), 7.43-7.54 (m, 2H), 7.39 (d, J=7.32 Hz, 1H), 7.25-7.35 (m, 3H), 7.14-7.22 (m, 1H), 5.26 (s, 2H), 3.52-3.68 (m, 6H), 2.40 (br s, 4H). MS: m/z=554.3 (M)+.

N-(3-Chloro-4-((3-fluorobenzypoxy)phenyl)-6-(4-(morpholinomethyl)phenyl)quinazolin-4-amine (23d). Yield: 36.8%. 1H NMR (500 MHz, DMSO-d6) δ: 9.92 (s, 1H), 8.80 (d, J=1.95 Hz, 1H), 8.60 (s, 1H), 8.20 (dd, J=1.95, 8.79 Hz, 1H), 8.04 (d, J=2.44 Hz, 1H), 7.84-7.87 (m, 3H), 7.77 (dd, J=2.69, 9.03 Hz, 1H), 7.46-7.54 (m, 3H), 7.28-7.36 (m, 3H), 7.20 (dt, J=1.95, 8.55 Hz, 1H), 5.28 (s, 2H), 3.61 (t, J=4.64 Hz, 4H), 3.56 (s, 2H), 2.40 (br s, 4H). MS: m/z=555.2 (M+H)+.

(3-(4-((3-Chloro-4-((3-fluorobenzyl)oxy)phenyl)amino)-quinazolin-6-yl)phenyl)(morpholino)methanone (23e). Yield: 53.2%. 1H NMR (500 MHz, DMSO-d6) δ: 9.99 (br s, 1H), 8.83 (d, J=1.46 Hz, 1H), 8.61 (s, 1H), 8.23 (dd, J=1.46, 8.79 Hz, 1H), 8.01 (d, J=2.44 Hz, 1H), 7.97 (d, J=7.81 Hz, 1H), 7.92 (s, 1H), 7.86 (d, J=8.79 Hz, 1H), 7.73 (dd, J=2.44, 8.79 Hz, 1H), 7.63 (t, J=7.81 Hz, 1H), 7.47 (q, J=7.65 Hz, 2H), 7.26-7.36 (m, 3H), 7.18 (dt, J=2.20, 8.67 Hz, 1H), 5.26 (s, 2H), 3.20-3.78 (m, 8H). MS: m/z=568.2 (M)+.

(4-(4-((3-Chloro-4-((3-fluorobenzyl)oxy)phenyl)amino)-quinazolin-6-yl)phenyl)(morpholino)methanone (23f). Yield: 58.9%. 1H NMR (500 MHz, DMSO-d6) δ: 9.93 (br s, 1H), 8.84 (br s, 1H), 8.60 (s, 1H), 8.21 (d, J=8.30 Hz, 1H), 7.93-8.05 (m, 3H), 7.86 (d, J=8.30 Hz, 1H), 7.76 (d, J=7.81 Hz, 1H), 7.60 (d, J=7.81 Hz, 2H), 7.43-7.51 (m, 1H), 7.26-7.36 (m, 3H), 7.18 (t, J=8.06 Hz, 1H), 5.26 (s, 2H), 3.37-3.80 (m, 8H). MS: m/z=568.2183 (M).

N-(3-Chloro-4-((3-fluorobenzyl)oxy)phenyl)-6-(3-(piperidin-1-yl)phenyl)quinazolin-4-amine (23g). Yield: 21%. 1H NMR (500 MHz, DMSO-d6) δ: 9.90 (s, 1H), 8.74 (d, J=1.95 Hz, 1H), 8.58 (s, 1H), 8.16 (dd, J=1.71, 8.55 Hz, 1H), 8.03 (d, J=2.44 Hz, 1H), 7.83 (d, J=8.79 Hz, 1H), 7.75 (dd, J=2.44, 8.79 Hz, 1H), 7.44-7.51 (m, 1H), 7.27-7.39 (m, 5H), 7.23 (d, J=7.81 Hz, 1H), 7.19 (dt, J=2.44, 8.55 Hz, 1H), 7.00 (dd, J=1.95, 8.30 Hz, 1H), 5.27 (s, 2H), 3.23-3.27 (m, 4H), 1.64-1.69 (m, 4H), 1.53-1.59 (m, 2H). MS: m/z=539.16 (M+H)+.

N-(3-Chloro-4-((3-fluorobenzyl)oxy)phenyl)-6-(4-(piperidin-1-yl)phenyl)quinazolin-4-amine (23h). Yield: 41.4%. 1H NMR (500 MHz, DMSO-d6) δ: 9.87 (s, 1H), 8.69 (d, J=0.98 Hz, 1H), 8.55 (s, 1H), 8.12 (dd, J=1.71, 8.55 Hz, 1H), 8.03 (d, J=2.44 Hz, 1H), 7.72-7.81 (m, 4H), 7.43-7.51 (m, 1H), 7.25-7.37 (m, 3H), 7.18 (dt, J=2.44, 8.55 Hz, 1H), 7.06 (d, J=8.79 Hz, 2H), 5.26 (s, 2H), 3.18-3.26 (m, 4H), 1.49-1.70 (m, 6H). MS: m/z=532.4 (M)+.

N-(3-Chloro-4-((3-fluorobenzypoxy)phenyl)-6-(3-(piperidin-1-ylmethyl)phenyl)quinazolin-4-amine (23i). Yield: 66.7%. 1H NMR (500 MHz, DMSO-d6) δ: 9.91 (br s, 1H), 8.81 (d, J=1.95 Hz, 1H), 8.60 (s, 1H), 8.19 (dd, J=1.95, 8.79 Hz, 1H), 8.01 (d, J=2.93 Hz, 1H), 7.87-7.97 (m, 3H), 7.74 (dd, J=2.90, 9.25 Hz, 1H), 7.63 (t, J=7.57 Hz, 1H), 7.52 (d, J=7.32 Hz, 1H), 7.46 (dt, J=6.35, 8.06 Hz, 1H), 7.26-7.35 (m, 3H), 7.17 (dt, J=2.44, 8.55 Hz, 1H), 5.25 (s, 2H), 4.22 (br s, 2H), 3.00 (d, J=5.86 Hz, 4H), 1.42-1.77 (m, 6H). MS: m/z=552.3 (M)+.

N-(3-Chloro-4-((3-fluorobenzypoxy)phenyl)-6-(4-(piperidin-1-ylmethyl)phenyl)quinazolin-4-amine (23j). Yield: 52.3%. 1H NMR (500 MHz, DMSO-d6) δ: 9.89 (br s, 1H), 8.80 (d, J=1.46 Hz, 1H), 8.58 (s, 1H), 8.19 (dd, J=1.95, 8.79 Hz, 1H), 8.01 (d, J=2.44 Hz, 1H), 7.90 (d, J=8.30 Hz, 2H), 7.85 (d, J=8.79 Hz, 1H), 7.74 (dd, J=2.44, 8.79 Hz, 1H), 7.56 (d, J=8.30 Hz, 2H), 7.41-7.50 (m, 1H), 7.25-7.35 (m, 3H), 7.17 (dt, J=2.20, 8.67 Hz, 1H), 5.25 (s, 2H), 3.93 (br s, 2H), 2.74 (br s, 4H), 1.56-1.69 (m, 4H), 1.45 (br s, 2H). MS: m/z=552.3 (M)+.

(3-(4-((3-Chloro-4-((3-fluorobenzyl)oxy)phenyl)amino)-quinazolin-6-yl)phenyl)(piperidin-1-yl)methanone (23k). Yield: 66.3%. 1H NMR (500 MHz, DMSO-d6) δ: 8.91 (s, 1H), 8.76 (br s, 1H), 8.35 (d, J=9.28 Hz, 1H), 7.96-8.00 (m, 2H), 7.87-7.93 (m, 2H), 7.70 (dd, J=2.45, 8.80 Hz, 1H), 7.59-7.65 (m, 2H), 7.44-7.52 (m, 2H), 7.32-7.36 (m, 3H), 7.20 (dt, J=2.44, 8.79 Hz, 1H), 5.30 (s, 2H), 3.65 (br s, 3H), 1.41-1.70 (m, 7H). MS: m/z=567.3 (M+H)+.

(4-(4-((3-Chloro-4-((3-fluorobenzyl)oxy)phenyl)amino)-quinazolin-6-yl)phenyl)(piperidin-1-yl)methanone (231). Yield: 73.3%. 1H NMR (500 MHz, DMSO-d6) δ: 9.93 (s, 1H), 8.83 (s, 1H), 8.59 (s, 1H), 8.21 (dd, J=1.95, 8.30 Hz, 1H), 8.01 (d, J=2.44 Hz, 1H), 7.93 (d, J=8.30 Hz, 2H), 7.86 (d, J=8.79 Hz, 1H), 7.74 (dd, J=2.44, 8.79 Hz, 1H), 7.53 (d, J=8.30 Hz, 3H), 7.43-7.49 (m, 1H), 7.27-7.34 (m, 3H), 7.17 (dt, J=1.95, 8.55 Hz, 1H), 5.25 (s, 2H), 3.60 (br s, 2H), 1.38-1.67 (m, 7H). MS: m/z=567.3 (M+H)+.

N-(4-(Benzyloxy)-3-chlorophenyl)-6-(3-morpholinophenyl)-quinazolin-4-amine (23m). Yield: 38%. 1H NMR (500 MHz, DMSO-d6) δ: 9.88 (s, 1H), 8.75 (d, J=1.95 Hz, 1H), 8.58 (s, 1H), 8.18 (dd, J=1.95, 8.79 Hz, 1H), 8.01 (d, J=2.93 Hz, 1H), 7.84 (d, J=8.30 Hz, 1H), 7.74 (dd, J=2.69, 9.03 Hz, 1H), 7.50 (d, J=7.32 Hz, 2H), 7.39-7.45 (m, 3H), 7.33-7.37 (m, 2H), 7.30 (d, J=8.79 Hz, 2H), 7.03 (dd, J=1.95, 8.30 Hz, 1H), 5.24 (s, 2H), 3.77-3.81 (m, 4H), 3.22-3.25 (m, 4H). MS: m/z=523.07 (M+H)+.

N-(4-(Benzyloxy)phenyl)-6-(3-morpholinophenyl)-quinazolin-4-amine (23n). Yield: 18%. 1H NMR (500 MHz, DMSO-d6) δ: 9.84 (s, 1H), 8.76 (d, J=1.46 Hz, 1H), 8.51 (s, 1H), 8.16 (dd, J=1.95, 8.79 Hz, 1H), 7.81 (d, J=8.30 Hz, 1H), 7.68 (d, J=9.30 Hz, 2H), 7.48 (d, J=7.32 Hz, 2H), 7.38-7.43 (m, 3H), 7.28-7.37 (m, 3H), 7.07 (d, J=8.80 Hz, 2H), 7.02 (dd, J=1.95, 7.80 Hz, 1H), 5.14 (s, 2H), 3.78 (t, J=4.87 Hz, 4H), 3.24 (t, J=4.87 Hz, 4H). MS: m/z=489.11 (M+H)+.

6-(3-Morpholinophenyl)-N-phenylquinazolin-4-amine (23o). Yield: 57.5%. 1H NMR (500 MHz, DMSO-d6) δ: 9.92 (s, 1H), 8.80 (d, J=1.46 Hz, 1H), 8.58 (s, 1H), 8.19 (dd, J=1.95, 8.79 Hz, 1H), 7.82-7.87 (m, 3H), 7.39-7.45 (m, 3H), 7.36 (d, J=2.44 Hz, 1H), 7.30-7.33 (m, 1H), 7.15-7.18 (m, 1H), 7.03 (dd, J=2.20, 8.06 Hz, 1H), 3.79 (t, J=4.85 Hz, 4H), 3.24 (t, J=4.85 Hz, 4H). MS: m/z=383.06 (M+H)+.

Aniline Synthesis. Anilines 17 were synthesized using general procedure B. In a 20 mL glass vial, a solution of substituted 4-nitrophenol (1.5 mmol) in 5 mL of acetonitrile was combined with potassium carbonate (3 mmol) and the appropriate benzyl bromide (1.5 mmol) at 25° C. The reaction mixture was stirred at 50° C. for 12 h. After completion of the alkylation reaction, the reaction mixture was triturated with 10 mL of 10% MeOH/DCM and was filtered through a silica gel plug. The organic filtrate was evaporated and redissolved in 5 mL of MeOH:H2O (5:1). To this solution was added zinc (0.29 g, 4.5 mmol), followed by ammonium chloride (0.48 g, 9 mmol) at 25° C. The temperature was raised to 50° C., and the stirring was continued for 6 h. Progress of the reaction was followed by LC-MS. Upon completion of the reaction, 15 mL of DCM:MeOH (1:1) was added to the reaction mixture and inorganic residues were removed by filtration. The filtrate was evaporated, and the residue was purified via flash chromatography (0-50% EtOAc/hexanes) to afford the desired substituted anilines.

4-(Benzyloxy)-3-chloroaniline (17e). Yield: 24.3%. 1H NMR (500 MHz, CDCl3) δ: 7.47 (d, J=7.32 Hz, 2H), 7.37-7.41 (m, 2H), 7.30-7.35 (m, 1H), 6.81 (d, J=8.79 Hz, 1H), 6.76 (d, J=2.93 Hz, 1H), 6.51 (dd, J=2.69, 8.55 Hz, 1H), 5.06 (s, 2H), 3.49 (br s, 2H). MS: m/z=234.02 (M+H)+. 4-((3-Bromobenzyl)oxy)-3-chloroaniline (17f). Yield: 25%. 1H NMR (500 MHz, CDCl3) δ: 7.62 (s, 1H), 7.45 (d, J=8.30 Hz, 1H), 7.39 (d, J=7.81 Hz, 1H), 7.22-7.26 (m, 1H), 6.75-6.80 (m, 2H), 6.52 (dd, J=2.93, 8.79 Hz, 1H), 5.01 (s, 2H), 3.52 (br s, 2H). MS: m/z=311.89 (M+H)+.

3-Chloro-4-((3-chlorobenzyl)oxy)aniline (17g). Yield: 38%. 1H NMR (500 MHz, CDCl3) δ: 7.47 (s, 1H), 7.29-7.36 (m, 3H), 6.78 (d, J=8.30 Hz, 1H), 6.75 (d, J=2.44 Hz, 1H), 6.50 (dd, J=2.93, 8.79 Hz, 1H), 5.01 (s, 2H), 3.52 (br s, 2H). MS: m/z=267.96 (M+H)+.

3-Chloro-4-((2,3-difluorobenzyl)oxy)aniline (17h). Yield: 31%. 1H NMR (500 MHz, CDCl3) δ: 7.35 (t, J=6.59 Hz, 1H), 7.07-7.16 (m, 2H), 6.83 (d, J=8.79 Hz, 1H), 6.75 (d, J=2.93 Hz, 1H), 6.52 (dd, J=2.69, 8.55 Hz, 1H), 5.12 (s, 2H), 3.51 (br s, 2H). MS: m/z=270.0 (M+H)+.

3-Chloro-4-((2-fluorobenzyl)oxy)aniline (17i). Yield: 37%. 1H NMR (500 MHz, CDCl3) δ: 7.58 (dt, J=1.71, 7.45 Hz, 1H), 7.27-7.32 (m, 1H), 7.16 (dt, J=0.98, 7.57 Hz, 1H), 7.04-7.08 (m, 1H), 6.83 (d, J=8.79 Hz, 1H), 6.75 (d, J=2.93 Hz, 1H), 6.51 (dd, J=2.69, 8.55 Hz, 1H), 5.12 (s, 2H), 3.50 (br s, 2H). MS: m/z=251.99 (M+H)+.

3-Chloro-4-((4-fluorobenzyl)oxy)aniline (17j). Yield: 22%. 1H NMR (500 MHz, CDCl3) δ: 7.40-7.44 (m, 2H), 7.03-7.10 (m, 2H), 6.78 (d, J=8.79 Hz, 1H), 6.75 (d, J=2.44 Hz, 1H), 6.50 (dd, J=2.93, 8.79 Hz, 1H), 5.00 (s, 2H), 3.50 (br s, 2H). MS: m/z=252.0 (M+H)+.

3-Chloro-4-((3-methoxybenzyl)oxy)aniline (17k). Yield: %. 1H NMR (500 MHz, CDCl3) δ: 7.27-7.32 (m, 1H), 7.01-7.05 (m, 2H), 6.86 (dd, J=2.69, 8.06 Hz, 1H), 6.79 (d, J=8.79 Hz, 1H), 6.74 (d, J=2.93 Hz, 1H), 6.49 (dd, J=2.45, 8.80 Hz, 1H), 5.04 (s, 2H), 3.83 (s, 3H), 3.44 (br s, 2H). MS: m/z=264.01 (M+H)+.

3-Chloro-4-((3-fluoro-4-(trifluoromethyl)benzyl)oxy)aniline (17l). Yield: 35.2%. 1H NMR (500 MHz, CDCl3) δ: 7.60 (t, J=7.81 Hz, 1H), 7.29-7.36 (m, 2H), 6.77 (d, J=4.39 Hz, 1H), 6.76 (d, J=1.47 Hz, 1H), 6.51 (dd, J=2.93, 8.79 Hz, 1H), 5.06 (s, 2H), 3.54 (br s, 2H). MS: m/z=319.93 (M+H)+.

3-Chloro-4-((2,3,5-trifluorobenzyl)oxy)aniline (17m). Yield: 22%. 1H NMR (500 MHz, CDCl3) δ: 7.13-7.18 (m, 1H), 6.85-6.93 (m, 1H), 6.81 (d, J=8.30 Hz, 1H), 6.76 (d, J=2.93 Hz, 1H), 6.53 (dd, J=2.93, 8.79 Hz, 1H), 5.09 (s, 2H), 3.54 (br s, 2H). MS: m/ z=287.96 (M+H)+.

4-((3-Fluorobenzyl)oxy)aniline (17n). Yield: 28%. 1H NMR (500 MHz, CDCl3) δ: 7.34 (m, 1H), 7.14-7.21 (m, 2H), 7.01 (dt, J=2.44, 8.55 Hz, 1H), 6.81 (d, J=8.30 Hz, 2H), 6.64 (d, J=8.30 Hz, 2H), 4.99 (s, 2H), 3.32 (br s, 2H). MS: m/z=218.07 (M+H)+.

4-((3-Fluorobenzyl)oxy)-3-methoxyaniline (17o). Yield: 7.52%. 1H NMR (500 MHz, CDCl3) δ: 7.28-7.33 (m, 1H), 7.14-7.21 (m, 2H), 6.97 (dt, J=2.20, 8.67 Hz, 1H), 6.70 (d, J=8.30 Hz, 1H), 6.32 (d, J=2.93 Hz, 1H), 6.16 (dd, J=2.69, 8.55 Hz, 1H), 5.02 (s, 2H), 3.84 (s, 3H), 3.51 (br s, 2H). MS: m/z=248.06 (M+H)+.

6-(4-(Morpholinosulfonyl)phenyl)quinazolin-4(3H)-one (18). A mixture of 6-iodoquinazolin-4(3H)-one (4.5 g, 16.54 mmol), (4- (morpholinosulfonyl)phenyl)boronic acid (4.93 g, 18.20 mmol), sodium carbonate (10.52 g, 99 mmol), and tetrakis-(triphenylphosphine)palladium(0) (1.338 g, 1.158 mmol) were combined in a flask, and 400 mL of 1,2-dimethoxyethane was added with ethanol (26.7 mL) and water (33.3 mL). The reaction mixture was heated with stirring at 80° C. for 30 h. The reaction progress was monitored by LC-MS. Upon completion of the reaction, the mixture was cooled to room temperature and the product precipitated. Solids were collected by filtration, washed with cold water, and air-dried, affording 18 (5.35 g, 14.40 mmol, 87% yield). 1H NMR (500 MHz, DMSO-d6) δ: 12.40 (br s, 1H), 8.44 (d, J=2.44 Hz, 1H), 8.22 (dd, J=2.20, 8.55 Hz, 1H), 8.16 (d, J=3.42 Hz, 1H), 8.07 (d, J=8.30 Hz, 2H), 7.84 (d, J=8.30 Hz, 2H), 7.80 (d, J=8.79 Hz, 1H), 3.64 (t, J=4.65 Hz, 4H), 2.91 (t, J=4.65 Hz, 4H). MS: m/z=372.2 (M+H)+.

4-((4-(4-Chloroquinazolin-6-yl)phenyl)sulfonyl)morpholine Hydrochloride (19). Thionyl chloride (9.83 mL, 135 mmol) was added slowly to 1.0 g of 18 (1 g, 2.69 mmol), followed by N,N dimethylformamide (2.085 μL, 0.027 mmol). The reaction mixture was refluxed for 36 h, monitoring reaction progress with LC-MS. The volatile components were removed via distillation, providing 19 (1.12 g, 1.683 mmol, 80% pure, 78% yield), which was used for subsequent reactions without further purification. 1H NMR (500 MHz, DMSOd6) δ: 8.45 (d, J=1.95 Hz, 1H), 8.32 (s, 1H), 8.26 (dd, J=2.20, 8.55 Hz, 1H), 8.07 (d, J=8.79 Hz, 2H), 7.79-7.86 (m, 3H), 3.65 (t, J=4.60 Hz, 4H), 2.92 (t, J=4.65 Hz, 4H). MS: m/z=390.04 (M+H)+.

6-(4-(Morpholinosulfonyl)phenyl)-N-arylquinazolin-4-amines hydrochloride 20 were synthesized following general procedure C. To a solution of 19 (100 μmol) in N,N-dimethylformamide (0.5 mL) was added aryl amine (110 μmol), and the mixture was heated on a shaker plate at 80° C. for 12 h. After cooling the reaction mixture to room temperature, 0.5 mL of 2-propanol was added. The resulting yellowish precipitate was filtered and washed with 2 mL of 2-propanol, affording the amines.

6-(4-(Morpholinosulfonyl)phenyl)-N-phenylquinazolin-4- amine Hydrochloride (20a). Yield: 50.8%. 1H NMR (500 MHz, DMSO-d6) δ: 11.51 (br s, 1H), 9.20 (s, 1H), 8.93 (s, 1H), 8.50 (d, J=8.79 Hz, 1H), 8.21 (d, J=8.30 Hz, 2H), 8.01 (d, J=8.30 Hz, 1H), 7.95 (d, J=8.30 Hz, 2H), 7.75 (d, J=8.30 Hz, 2H), 7.51-7.55 (m, 2H), 7.34-7.37 (m, 1H), 3.65-3.67 (br m, 4H), 2.92-2.95 (br m, 4H). MS: m/z=447.2 (M+H)+.

6-(4-(Morpholinosulfonyl)phenyl)-N-(p-tolyl)quinazolin-4-amine Hydrochloride (20b). Yield: 54.9%. 1H NMR (500 MHz, DMSO-d6) δ: 11.62 (br s, 1H), 9.24 (s, 1H), 8.93 (s, 1H), 8.51 (d, J=8.30 Hz, 1H), 8.22 (d, J=8.30 Hz, 3H), 8.03 (d, J=8.79 Hz, 1H), 7.95 (d, J=8.79 Hz, 2H), 7.63 (d, J=7.81 Hz, 2H), 7.34 (d, J=7.81 Hz, 2H), 3.67 (t, J=4.4 Hz, 4H), 2.95 (t, J=4.4 Hz, 4H), 2.38 (s, 3H). MS: m/z=461.2 (M+H)+.

2-Chloro-4-((6-(4-(morpholinosulfonyl)phenyl)quinazolin-4-yl)amino)phenol Hydrochloride (20c). Yield: 52.7%. 1H NMR (500 MHz, DMSO-d6) δ: 11.60 (br s, 1H), 10.55 (br s, 1H), 9.22 (s, 1H), 8.96 (s, 1H), 8.51 (dd, J=1.47, 8.79 Hz, 1H), 8.22 (d, J=8.30 Hz, 2H), 8.02 (d, J=8.79 Hz, 1H), 7.95 (d, J=8.30 Hz, 2H), 7.81 (d, J=2.93 Hz, 1H), 7.52 (dd, J=2.44, 8.79 Hz, 1H), 7.12 (d, J=8.79 Hz, 1H), 3.7 (t, J=4.40 Hz, 4H), 2.95 (t, J=4.4 Hz, 4H). MS: m/z=497.2 (M+H)+.

N-(3-Chloro-4-methoxyphenyl)-6-(4-(morpholinosulfonyl)-phenyl)quinazolin-4-amine Hydrochloride (20d). Yield: 43.4%. 1H NMR (500 MHz, DMSO-d6) δ: 11.34 (br s, 1H), 9.14 (s, 1H), 8.94 (s, 1H), 8.49 (d, J=8.79 Hz, 1H), 8.20 (d, J=8.79 Hz, 2H), 8.00 (d, J=8.79 Hz, 1H), 7.96 (d, J=8.30 Hz, 2H), 7.93 (d, J=2.44 Hz, 1H), 7.70 (dd, J=2.44, 8.79 Hz, 1H), 7.31 (d, J=9.28 Hz, 1H), 3.93 (s, 3H), 3.67 (t, J=4.6 Hz, 4H), 2.95 (t, J=4.6 Hz, 4H). MS: m/z=511.1 (M+H)+.

N-(4-(Benzyloxy)-3-chlorophenyl)-6-(4-(morpholinosulfonyl)phenyl)quinazolin-4-amine Hydrochloride (20e). Yield: 70.1%. 1H NMR (500 MHz, DMSO-d6) δ: 11.49 (br s, 1H), 9.18 (s, 1H), 8.95 (s, 1H), 8.50 (dd, J=1.71, 8.55 Hz, 1H), 8.20-8.22 (m, 2H), 8.01 (d, J=8.79 Hz, 1H), 7.93-7.96 (m, 3H), 7.68 (dd, J=2.69, 9.03 Hz, 1H), 7.51-7.53 (m, 2H), 7.35-7.46 (m, 4H), 5.30 (s, 2H), 3.66-3.68 (t, J=4.9 Hz, 4H), 2.5 (t, J=4.4 Hz, 4H). MS: m/z=587.2 (M+H)+.

N-(4-((3-Bromobenzyl)oxy)-3-chlorophenyl)-6-(4-(morpholinosulfonyl)phenyl)quinazolin-4-amine Hydrochloride (20f). Yield: 51.6%. 1H NMR (500 MHz, DMSO-d6) δ: 11.45 (br s, 1H), 9.17 (s, 1H), 8.95 (s, 1H), 8.50 (d, J=8.79 Hz, 1H), 8.21 (d, J=8.79 Hz, 2H), 8.01 (d, J=8.30 Hz, 1H), 7.94-7.97 (m, 3H), 7.72 (s, 1H), 7.69 (dd, J=2.69, 9.03 Hz, 1H), 7.58 (d, J=7.81 Hz, 1H), 7.52 (d, J=7.81 Hz, 1H), 7.37-7.43 (m, 2H), 5.31 (s, 2H), 3.67 (t, J=4.4 Hz, 4H), 2.95 (t, J=4.35 Hz, 4H). MS: m/z=665.1 (M+H)+.

N-(3-Chloro-4-((3-chlorobenzyl)oxy)phenyl)-6-(4-(morpholinosulfonyl)phenyl)quinazolin-4-amine Hydrochloride (20g). Yield: 49.1%. 1H NMR (500 MHz, DMSO-d6) δ: 11.54 (br s, 1H), 9.19 (br s, 1H), 8.96 (br s, 1H), 8.50 (d, J=8.79 Hz, 1H), 8.21 (d, J=8.30 Hz, 2H), 7.98-8.08 (m, 1H), 7.94-7.96 (m, 3H), 7.69 (dd, J=2.44, 8.79 Hz, 1H), 7.58 (br s, 1H), 7.37-7.50 (m, 4H), 5.31 (br s, 2H), 3.67 (br s, 4H), 2.95 (br s, 4H). MS: m/z=621.1 (M+H)+.

N-(3-Chloro-4-((2,3-difluorobenzyl)oxy)phenyl)-6-(4-(morpholinosulfonyl)phenyl)quinazolin-4-amine Hydrochloride (20h). Yield: 53.3%. 1H NMR (500 MHz, DMSO-d6) δ: 11.58 (br s, 1H), 9.22 (s, 1H), 8.97 (s, 1H), 8.51 (dd, J=1.46, 8.79 Hz, 1H), 8.22 (d, J=8.30 Hz, 2H), 8.03 (d, J=8.79 Hz, 1H), 7.93-7.97 (m, 3H), 7.73 (dd, J=2.44, 8.79 Hz, 1H), 7.43-7.53 (m, 3H), 7.28-7.34 (m, 1H), 5.38 (s, 2H), 3.67 (t, J=4.4 Hz, 4H), 2.95 (t, J=4.6 Hz, 4H). MS: m/z=623.2 (M+H)+.

N-(3-Chloro-4-((2-fluorobenzypoxy)phenyl)-6-(4-(morpholinosulfonyl)phenyl)quinazolin-4-amine Hydrochloride (20i). Yield: 41.2%. 1H NMR (500 MHz, DMSO-d6) δ: 11.32 (br s, 1H), 9.12 (br s, 1H), 8.93 (br s, 1H), 8.47 (d, J=8.79 Hz, 1H), 8.18 (d, J=8.30 Hz, 2H), 7.98 (d, J=8.79 Hz, 1H), 7.92-7.96 (m, 3H), 7.70 (dd, J=2.44, 8.79 Hz, 1H), 7.60-7.63 (m, 1H), 7.43-7.46 (m, 2H), 7.25-7.32 (m, 2H), 5.31 (s, 2H), 3.65 (t, J=4.6 Hz, 4H), 2.93 (t, J=4.6 Hz, 4H). MS: m/z=605.2 (M+H)+.

N-(3-Chloro-4-((4-fluorobenzyl)oxy)phenyl)-6-(4-(morpholinosulfonyl)phenyl)quinazolin-4-amine Hydrochloride (20j). Yield: 67.1%. 1H NMR (500 MHz, DMSO-d6) δ: 11.66 (br s, 1H), 9.24 (br s, 1H), 8.97 (s, 1H), 8.52 (d, J=8.79 Hz, 1H), 8.23 (d, J=8.30 Hz, 2H), 8.03 (d, J=8.79 Hz, 1H), 7.94-7.96 (m, 3H), 7.70 (dd, J=2.44, 8.79 Hz, 1H), 7.55-7.58 (m, 2H), 7.41 (d, J=8.79 Hz, 1H), 7.26-7.30 (m, 2H), 5.28 (s, 2H), 3.7 (t, J=4.9 Hz, 4H), 2.95 (t, J=4.6 Hz, 4H). MS: m/z=605.2 (M+H)+.

N-(3-Chloro-4-((3-methoxybenzyl)oxy)phenyl)-6-(4-(morpholinosulfonyl)phenyl)quinazolin-4-amine Hydrochloride (20k). Yield: 43.1%. 1H NMR (500 MHz, DMSO-d6) δ: 11.40 (br s, 1H), 9.15 (s, 1H), 8.94 (s, 1H), 8.49 (d, J=8.30 Hz, 1H), 8.20 (d, J=8.30 Hz, 2H), 8.00 (d, J=8.79 Hz, 1H), 7.93-7.97 (m, 3H), 7.68 (dd, J=2.44, 8.79 Hz, 1H), 7.33-7.39 (m, 2H), 7.06-7.08 (m, 2H), 6.91-6.96 (m, 1H), 5.27 (s, 2H), 3.78 (s, 3H), 3.67 (t, J=4.9 Hz, 4H), 2.95 (t, J=4.4 Hz, 4H). MS: m/z=617.2 (M+H)+.

N-(3-Chloro-4-((3-fluoro-4-(trifluoromethyl)benzyl)oxy)-phenyl)-6-(4-(morpholinosulfonyl)phenyl)quinazolin-4-amine Hydrochloride (201). Yield: 40.9%. 1H NMR (500 MHz, DMSO-d6) δ: 11.74 (br s, 1H), 9.29 (s, 1H), 8.98 (s, 1H), 8.52 (dd, J=1.46, 8.79 Hz, 1H), 8.24 (d, J=8.79 Hz, 2H), 8.05 (d, J=8.79 Hz, 1H), 7.99 (d, J=2.44 Hz, 1H), 7.95 (d, J=8.30 Hz, 2H), 7.89 (t, J=7.81 Hz, 1H), 7.72 (dd, J=2.44, 8.79 Hz, 1H), 7.63 (d, J=11.23 Hz, 1H), 7.55 (d, J=7.81 Hz, 1H), 7.38 (d, J=8.79 Hz, 1H), 5.43 (s, 2H), 3.67 (t, J=4.4 Hz, 4H), 2.95 (t, J=4.4 Hz, 4H). MS: m/z=673.2 (M+H)+.

N-(3-Chloro-4-((2,3,5-trifluorobenzyl)oxy)phenyl)-6-(4-(morpholinosulfonyl)phenyl)quinazolin-4-amine Hydrochloride (20m). Yield: 42%. 1H NMR (500 MHz, DMSO-d6) δ: 11.49 (br s, 1H), 9.19 (s, 1H), 8.96 (s, 1H), 8.51 (d, J=9.77 Hz, 1H), 8.21 (d, J=8.30 Hz, 2H), 8.02 (d, J=8.79 Hz, 1H), 7.95-7.98 (m, 3H), 7.73 (dd, J=2.69, 9.03 Hz, 1H), 7.56-7.65 (m, 1H), 7.48 (d, J=8.79 Hz, 1H), 7.34-7.36 (m, 1H), 5.38 (s, 2H), 3.67 (t, J=4.9 Hz, 4H), 2.95 (t, J=4.4 Hz, 4H). MS: m/z=641.1 (M+H)+.

N-(4-((3-Fluorobenzyl)oxy)phenyl)-6-(4-(morpholinosulfonyl)phenyl)quinazolin-4-amine (20n). Yield: 45.8%. 1H NMR (500 MHz, DMSO-d6) δ: 11.40 (br s, 1H), 9.15 (s, 1H), 8.88 (s, 1H), 8.49 (d, J=8.79 Hz, 1H), 8.20 (d, J=8.79 Hz, 2H), 7.92-8.02 (m, 3H), 7.65 (d, J=9.30 Hz, 2H), 7.45-7.51 (m, 1H), 7.31-7.35 (m, 2H), 7.16-7.22 (m, 3H), 5.21 (s, 2H), 3.67 (t, J=4.4 Hz, 4H), 2.95 (t, J=4.4 Hz, 4H). MS: m/z=571.2 (M+H)+.

N-(4-((3-Fluorobenzyl)oxy)-3-methoxyphenyl)-6-(4-(morpholinosulfonyl)phenyl)quinazolin-4-amine (20o). Yield: 18%. 1H NMR (500 MHz, DMSO-d6) δ: 11.32 (br s, 1H), 9.14 (s, 1H), 8.90 (s, 1H), 8.49 (d, J=8.30 Hz, 1H), 8.20 (d, J=8.79 Hz, 2H), 7.99 (d, J=8.79 Hz, 1H), 7.96 (d, J=8.30 Hz, 2H), 7.45-7.50 (m, 1H), 7.41 (d, J=2.44 Hz, 1H), 7.28-7.34 (m, 3H), 7.15-7.22 (m, 2H), 5.19 (s, 2H), 3.84 (s, 3H), 3.67 (t, J=4.9 Hz, 4H), 2.95 (t, J=4.4 Hz, 4H). MS: m/z=601.1 (M+H)+.

### Trypanosome Replication Assays.

Bloodstream T. brucei brucei Lister 427 cells were seeded at a density of 2×103 cells/mL and cultured in HMI-9 medium in a 24-well plate. Then 2 μL of DMSO (control) or different concentrations of drugs prepared from 200× DMSO stocks were added to the cultures. Cells were incubated at 37° C. for 48 h and counted with a hemocytometer. Drugs were initially tested at concentrations of 10 μM, 1 μM, 100 nM, and 10 nM to determine the range of potency of each compound. Thereafter, a set of five concentrations centered around a dose that prevented replication of 50% of cells (compared to DMSO) was used to establish the EC50. All experiments were repeated thrice in independent studies where each dose was administered in duplicate (total n=6). HepG2 Cell Toxicity Assay. The 384 well MTT cytotoxicity assay is a modification of the MTT method described by Ferrari et al. optimized for 384-well throughput, with modifications described below. The 50% inhibitory concentrations (IC50) were generated for each toxicity dose response test using GraphPad Prism (GraphPad Software Inc., San Diego, Calif.) using the nonlinear regression (sigmoidal dose-response/variable slope) equation.

Biological Assay Details.

HepG2 Cell Toxicity assay. The 384 well MTT cytotoxicity assay is a modification of the MTT method described by Ferrari et al.¹ ENREF_{—}26_ENREF_{—}26_ENREF_{—}26 optimized for 384 well throughput. HepG2 cells were cultured in complete Minimal Essential Medium (Minimum Essential Medium (Gibco-Invitrogen, #11090-099) prepared by supplementing MEM with 0.19% sodium bicarbonate (Gibco-BRL Cat #25080-094), 10% heat inactivated FBS (Gibco-Invitrogen #16000-036), 2 mM L-glutamine (Gibco-Invitrogen #25030-081), 0.1 mM MEM non-essential amino acids (Gibco-Invitrogen #11140-050), 0.009 mg/ml insulin (Sigma #I1882), 1.76 mg/ml bovine serum albumin (Sigma #A1470), 20 units/ml penicillin-streptomycin (Gibco-Invitrogen #15140-148), and 0.05 mg/ml gentamycin (Gibco-Invitrogen #15710-064). HepG2 cells cultured in complete MEM were first washed with 1× Hank's Balanced Salt Solution (Invitrogen #14175-095), trypsonized using a 0.25% trypsin/EDTA solution (Invitrogen #25200-106), assessed for viability using trypan blue, and resuspended at 250,000 cells/ml. Using a Tecan EVO Freedom robot, 38.3 μL of cell suspension were added to each well of clear, cell culture-treated 384-well microtiter plates (Nunc Cat#164688) for a final concentration of 9570 liver cells per well, and plated cells were incubated overnight in 5% CO2 at 37° C. Drug plates were prepared with the Tecan EVO Freedom using sterile 96 well plates containing twelve duplicate 1.6-fold serial dilutions of each test compound suspended in DMSO. 4.25 μL of diluted test compound was then added to the 38.3 μL of media in each well providing a 10 fold final dilution of compound. Compounds were tested from a range of 57 ng/ml to 10,000 ng/ml for all assays. Mefloquine was used as a plate control for all assays with a concentration ranging from 113 ng/ml to 20,000 ng/ml. After a 48 hour incubation period, 8 μL of a 1.5 mg/ml solution of MTT diluted in complete MEM media was added to each well. All plates were subsequently incubated in the dark for 1 hour at room temperature. After incubation, the media and drugs in each well was removed by shaking plate over sink, the plates are then left to dry in hood for 15 minutes. Next, 304 of isopropanol acidified by addition of HCl at a final concentration of 0.36% was added to dissolve the formazan dye crystals created by reduction of MTT. Plates are put on a 3-D rotator for 15-30 minutes. Absorbance was determined in all wells using a Tecan iControl 1.6 Infinite plate reader. The 50% inhibitory concentrations (IC50s) were then generated for each toxicity dose response test using GraphPad Prism (GraphPad Software Inc., San Diego, Calif.) using the nonlinear regression (sigmoidal dose-response/variable slope) equation.

### Pharmacokinetic Analysis.

Test system. Healthy male BALB/c mice (8-12 weeks old) weighing between 20 to 35 g were procured from In vivo biosciences, Bengaluru, India. Maximum three mice were housed in each polycarbonate cage. Temperature and humidity were maintained at 22±3° C. and 40-70%, respectively and illumination was controlled to give a sequence of 12 hr light and 12 hr dark cycle. The temperature and humidity were recorded by auto-controlled data logger system. Animals were provided laboratory rodent diet (Vetcare India Pvt. Ltd, Bengaluru) ad libitum and were provided fresh water ad libitum (reverse osmosis water treated with UV light).

Study Design. Twenty seven male BALB/c mice were weighed and administered orally with NEU-617 suspension formulation at a dose of 40 mg/kg. The dosing volume administered for was 10 mL/kg.

Formulation Preparation. Formulation of NEU-617 was given orally. The strength of oral suspension formulation was 4 mg/mL. The weighed quantity (39.22 mg) of compound NEU-617 for p.o. dosing was added in a mortar. The volume of 0.01 mL of Tween 80 and 9.795 mL of 0.5% NaCMC in water was added with continuous trituration, this prepared suspension was then transferred to labeled bottle and vortexed for 2 minutes followed by sonication for 2 minutes to obtain homogenous suspension.

Formulation results. After preparation of formulations for dosing, a volume of 200 μL was aliquoted for analysis. The formulation was analyzed and the concentration was found to be 4.185 mg/ml, which is within the acceptance criteria (in-house acceptance criteria is ±20% from the nominal value).

Sample Collection. Blood samples were collected from a set of three mice at each time point [pre-dose, 0.25, 0.5, 1, 2, 4, 6, 8 and 24 hr]. The blood samples (approximately 60 μL) were collected from the retro-orbital plexus into labeled tubes, containing K₂EDTA solution, as an anticoagulant. Plasma was harvested from the blood by centrifugation at 4000 rpm for 10 min at 4±2° C. and stored below −70° C. until bioanalysis. After collecting the blood samples, mice were humanely euthanized by CO₂ asphyxiation and brain was collected at pre-dose, 0.25, 0.5, 1, 2, 4, 6, 8 and 24 hr. Following collection, the brain samples were washed by dipping in 20 mL of ice-cold phosphate buffer saline (pH 7.4), dried gently on a filter paper, weighed and placed in polypropylene tubes. Further brain samples were homogenized using ice-cold phosphate buffer saline (pH 7.4) and the total homogenate volume was thrice the brain weight. The brain samples were stored at −70° C. until bioanalysis.

Data analysis. The Non-Compartmental-Analysis module in WinNonlin® (Version 5.2) was used to assess the pharmacokinetic parameters. Peak plasma concentrations (Cmax) and time for the peak plasma concentrations (Tmax) were the observed values. The areas under the concentration time curve (AUClast and AUCinf) were calculated by linear trapezoidal rule.

TABLE 15
Pharmacokinetic parameters of NEU-617 (23a) in plasma and
brain following a single oral administration in male BALB/c
mice (Dose: 40 mg/kg)

| Dose | | | | | | |
|---|---|---|---|---|---|---|
| (mg/kg) | Route | Matrix | Tₘₐₓ(h) | Cₘₐₓ(ng/mL) | AUCₗₐₛₜ | AUC_{inf} |
| 40 | PO | Plasma | 2.00 | 152.62 | 1952.07 | 1998.63 |
| | | Brain | 0.50 | 26.0 | 92.48 | Not |
| | | | | | | calc |
| | | | Brain:Plasma | 0.17 | 0.05 | |

### Plasma Protein Binding Experiments.

Mice plasma (BALB/c mice, male—pool of 3 animals) was collected in-house using K₂EDTA as anticoagulant. Warfarin (Lot #376-34A, 99.5%) was procured from Supelco, West Chester, Pa. Glipizide (Cat #50402G) was purchased from Apin chemicals, Abingdon, Oxon. Albendazole (Cat #A4673) was purchased from Sigma. Phosphate buffered saline pH 7.4 (0.1 M sodium phosphate and 0.15 M sodium chloride) and RED device inserts were procured from Thermo Scientific, Meridian Rd., Rockford, Ill. DMSO (Cat #D5879, >99.5% pure) were procured from Sigma, Germany.

From 20 mM DMSO stock sub stock of 1 mM was prepared in DMSO for 23a and warafarin. Further 1 mM stock was diluted 200-fold in mice plasma to prepare a concentration of 5 μM. The final DMSO concentration in plasma was 0.5%.

Rapid equilibrium dialysis was performed with a rapid equilibrium dialysis (RED) device containing dialysis membrane with a molecular weight cut-off of 8,000 Daltons. Each dialysis insert contains two chambers. The red chamber is for plasma while the white chamber is for buffer. A 200 μL aliquot of warfarin or test compound at 5 μM (triplicates) was separately added to the plasma chamber and 350 μL of phosphate buffer saline (pH 7.4) was added to the buffer chamber of the inserts. After sealing the RED device with an adhesive film, dialysis was performed at 37° C. with shaking at 100 rpm for 4 hours as per manufacturer's recommendation.

Recovery and stability: A 50 μL aliquot of warfarin or test compound was added to four 0.5 mL microfuse tubes. Two aliquots were frozen immediately (0 minute sample). The other two aliquots were incubated at 37° C. for 4 hours along with the RED device.

Following dialysis, an aliquot of 50 μL was removed from each well (both plasma and buffer side) and diluted with equal volume of opposite matrix (dialyzed with the other matrix) to nullify the matrix effect. Similarly, 50 μL of buffer was added to recovery and stability samples. An aliquot of 100 μL was submitted for LC-MS/MS analysis.

A 25 μL aliquot of warfarin and test compounds were crashed with 100 μL of acetonitrile containing internal standard (glipizide for warfarin and test compound) and vortexed for 5 minutes. The samples were centrifuged at 15,000 rpm at 4° C. for 10 min and 100 μL of supernatant was submitted for LCMS/MS analysis. Samples were monitored for parent compound in MRM mode using LC-MS/MS. The LC-MS/MS conditions and MRM chromatogram are summarized in Table 16.

TABLE 16
Plasma protein binding results.

| | % Bound | | | | % | % compound |
|---|---|---|---|---|---|---|
| Compound | R1 | R2 | R3 | Mean (SD) | recovery | remaining at 4 h |
| Warfarin | 94.9 | 94.2 | 94.1 | 84.4 (0.5) | 95 | 100 |
| 23a | 99.5 | 99.7 | 99.6 | 99.6 (0.01) | 100 | 85 |

### Drug Efficacy in a Mouse Model of Human African Trypanosomiasis:

Swiss Webster (female) mice, aged 8-10 weeks, were intraperitoneally inoculated with 10³ T. brucei CA427 strain using a 1 ml hypodermic syringe and 26G ½″ needle (day 0). The mice were assigned to control or NEU617 -treated groups (four per group). Treatment was initiated one-day post infection for two weeks. Control group received the vehicle only. Drug-treated mice were given 10 mg/kg NEU617 twice daily at 12 h interval (orally or intraperitoneally) based on their body weights. NEU617 was reconstituted in (i) DMSO for intraperitoneal (I.P.) administration, and (ii) N-methyl-2-pyrrolidone and polyethylene glycol 300 (1:9, v/v) for oral (P.O.) dosing. Drugs were prepared fresh daily and the concentration was adjusted so that the animals received <200 μl of solvent/dose/animal. Parasitemia was monitored daily by collecting 2 μl of blood (tail prick) into 18 μl of 0.85% ammonium chloride (NH₄Cl) (1:10 dilution) and counting the parasites with a hemocytometer. Further dilutions of blood were made with bicine-buffered saline with glucose, as needed. Animals showing distress symptoms at any stage of the study were euthanized. All experiments were conducted following protocols approved by the Institutional Animal Care and Use Committee (IACUC).

### Additional Synthesis and Compounds:

Chemical synthesis. Unless otherwise noted, reagents were obtained from Sigma-Aldrich, Inc. (St. Louis, Mo.), Fisher Scientific, Frontier Scientific Services, Inc. (Newark, Del.), Matrix Scientific (Columbia, S.C.) and used as received. Boronic acids/esters and aniline reagents were typically purchased. Reaction solvents were purified by passage through alumina columns on a purification system manufactured by Innovative Technology (Newburyport, Mass.). Microwave reactions were performed using a Biotage Initiatior-8 instrument. NMR spectra were obtained with Varian NMR systems, operating at 400 or 500 MHz for ¹H acquisitions as noted. LCMS analysis was performed using a Waters Alliance reverse-phase HPLC, with single-wavelength UV-visible detector and LCT Premier time-of-flight mass spectrometer (electrospray ionization). All newly synthesized compounds were that were submitted for biological testing were deemed >95% pure by LCMS analysis (UV and ESI-MS detection) prior to submission for biological testing. Preparative LCMS was performed on a Waters FractionLynx system with a Waters MicroMass ZQ mass spectrometer (electrospray ionization) and a single-wavelength UV-visible detector, using acetonitrile/H₂O gradients with 0.1% formic acid. Fractions were collected on the basis of triggering using UV and mass detection. Yields reported for products obtained by preparative HPLC represent the amount of pure material isolated; impure fractions were not repurified.

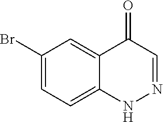 [SMILES: O=c1cn[nH]c2ccc(Br)cc12]

6-Bromocinnolin-4(1H)-one (15). In a 250 mL round bottom flask was added 1-(2-amino-5-bromophenyl)ethanone (8.34 g, 39.0 mmol), water (30 mL), and conc. hydrochloric acid (30 mL, 987 mmol). The mixture was cooled to 0° C. in an ice bath and allowed to stir for 15 minutes until a suspension resulted. Aqueous sodium nitrite (2M, 20 mL, 40.0 mmol) was then added dropwise with an addition funnel. The resulting solution was allowed to warm to room temperature over 1.5 hours and was stirred at room temperature overnight, then refluxed for 6 hours. The mixture was cooled to room temperature, water (200 mL) was added, and the mixture was extracted with ethyl acetate (3×200 mL). The combined organic layers were then washed with brine (50 mL), dried over sodium sulfate, filtered, and concentrated on to silica. The crude product was then purified by flash column chromatography using a gradient of 1-10% methanol in dichloromethane to yield 15 as a dark brown solid in 82% yield. ¹H NMR (500 MHz, DMSO-d₆) δ 14.09 (br. s., 1H), 8.09 (d, J=2.2 Hz, 1H), 7.92 (dd, J=8.8, 2.2 Hz, 1H), 7.79 (s, 1H), 7.71 (d, J=9.1 Hz, 1H). LCMS found 224.9 [M+H]⁺.

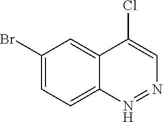 [SMILES: Clc1cnnc2ccc(Br)cc12]

6-Bromo-4-chlorocinnoline (8a) In a flame dried 250 mL round bottom flask was added 6-bromocinnolin-4(1H)-one (1.00 g, 4.44 mmol), anhydrous tetrahydrofuran (45 mL), and phosphorus oxychloride (1.25 mL, 13.41 mmol). The mixture was refluxed for 1 hour at which point a deep green/blue solution had resulted. The solution was cooled to 0° C. and was quenched by the dropwise addition of sat. aq. NaHCO₃ (70 mL). The mixture was allowed to warm to room temperature and stir for an additional 1 hour. Water (50 mL) was added and the mixture was extracted with dichloromethane (3×100 mL). The combined organic layers were washed with sat. aq. NaHCO₃ (50 mL), washed with brine (50 mL), dried over Na₂SO₄, and concentrated on to silica, and purified by flash column chromatography using a gradient of 1-5% methanol in dichloromethane to yield an inseparable 10:1 mixture of 8a and 8b as a brown solid in 85% yield. ¹H NMR (500 MHz, CDCl₃) δ 9.36 (s, 1H), 8.43 (d, J=8.8 Hz, 1H), 8.36 (d, J=2.0 Hz, 1H), 7.98 (dd, J=9.3, 2.0 Hz, 1H). LCMS found 242.9/199.0 [M+H]⁻.

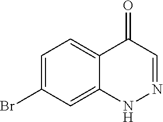 [SMILES: O=c1cn[nH]c2cc(Br)ccc12]

7-Bromocinnolin-4(1H)-one (18) In a 50 mL round bottom flask was added 1-(2-amino-4-bromophenyl)ethanone (712 mg, 3.33 mmol), water (3 mL), and conc. hydrochloric acid (3 mL, 99 mmol). The mixture was cooled to 0° C. in an ice bath and allowed to stir for 15 minutes until a suspension resulted. Aqueous sodium nitrite (2M, 1.84 mL, 3.68 mmol) was then added dropwise with an addition funnel. The resulting solution was allowed to warm to room temperature over 1.5 hours and was stirred at room temperature overnight, then refluxed for 6 hours. The mixture was cooled to room temperature, water (35 mL) was added, and the mixture was extracted with ethyl acetate (3×40 mL). The combined organic layers were then washed with brine (20 mL), dried over sodium sulfate, filtered, and concentrated on to silica. The crude product was then purified by flash column chromatography using a gradient of 20-50% ethyl acetate in hexanes, then ethyl acetate to yield 18 as a light brown solid in 26% yield. ¹H NMR (500 MHz, DMSO-d₆) δ 13.49 (s, 1H), 7.92 (d, J=8.8 Hz, 1H), 7.76 (s, 1H), 7.73 (d, J=2.0 Hz, 1H), 7.53 (dd, J=8.8, 2.0 Hz, 1H). LCMS found 225.0 [M+H]⁺.

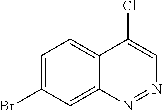 [SMILES: Clc1cnnc2cc(Br)ccc12]

7-Bromo-4-chlorocinnoline (8c) In a flame dried 25 mL round bottom flask was added 7-bromocinnolin-4(1H)-one (166 mg, 0.74 mmol), anhydrous tetrahydrofuran (7 mL), and phosphorus oxychloride (0.2 mL, 2.15 mmol). The mixture was refluxed for 1 hour at which point a deep green/blue solution had resulted. The solution was cooled to 0° C. and was quenched by the dropwise addition of sat. aq. NaHCO₃ (12 mL). The mixture was allowed to warm to room temperature and stir for an additional 1 hour. Water (12 mL) was added and the mixture was extracted with dichloromethane (3×25 mL). The combined organic layers were washed with sat. aq. NaHCO₃ (20 mL), washed with brine (20 mL), dried over Na₂SO₄, and to yield 8c as a dark brown solid in 92% yield. ¹H NMR (500 MHz, CDCl₃) δ 9.39 (s, 1H), 8.76 (d, J=2.0 Hz, 1H), 8.09 (d, J=9.3 Hz, 1H), 7.95 (dd, J=9.0, 1.7 Hz, 1H). LCMS found 242.9 [M+H]⁺.

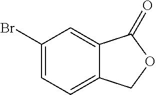 [SMILES: O=C1OCc2ccc(Br)cc21]

6-Bromoisobenzofuran-1(3H)-one (20a) In a 100 mL round bottom flask was dissolved isobenzofuran-1(3H)-one (4.01 g, 29.9 mmol) in trifluoroacetic acid (14 mL, 182 mmol) and sulfuric acid (6.5 mL, 122 mmol). N-Bromosuccinimide (7.95 g, 1.49 mmol) was added portionwise over 8 hours and the solution was stirred at room temperature for an additional 87 hours. The solution was diluted with water (40 mL) and ethyl acetate (40 mL). The pH of the aqueous layer was neutralized with 1M aq. NaOH and sat. aq. NaHCO₃. The organic layer was separated and the aqueous layer was extracted with ethyl acetate (3×50 mL). The combined organic layers were washed with brine (25 mL), dried over Na₂SO₄, and concentrated on to silica. The crude product was then purified by flash column chromatography using 10-20% ethyl acetate in hexanes to yield 20a as white solid in 57% yield. ¹H NMR (500 MHz, CDCl₃) δ 7.98 (d, J=1.5 Hz, 1H), 7.77 (dd, J=8.3, 1.5 Hz, 1H), 7.40 (d, J=8.3 Hz, 1H), 5.27 (s, 2H). LCMS found 212.9 [M+H]⁺.

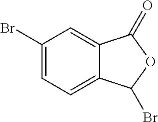 [SMILES: O=C1OC(Br)c2ccc(Br)cc21]

3,6-Dibromoisobenzofuran-1(3H)-one (21a) In a 50 mL round bottom flask was added 6-bromoisobenzofuran-1(3H)-one (1.00 g, 4.69 mmol), N-bromosuccinimide (958 mg, 5.38 mmol), 2,2′-azobis(2-methylpropionitrile) (75 mg, 0.46 mmol), and chloroform (23 mL). The mixture was refluxed for 2.5 hours, then cooled to room temperature and quenched with sat. aq. NaHCO₃ (25 mL). The organic layer was removed, washed with water (20 mL), washed with brine (15 mL), and concentrated on to silica. The crude product was purified by flash column chromatography using a gradient of 5-10% ethyl acetate in hexanes to yield 21a as a white solid in 61% yield. ¹H NMR (500 MHz, CDCl₃) δ 8.06 (d, J=1.5 Hz, 1H), 7.90 (dd, J=8.1, 1.7 Hz, 1H), 7.52 (d, J=8.3 Hz, 1H), 7.37 (s, 1H). LCMS does not ionize.

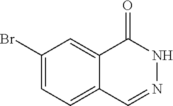 [SMILES: O=c1[nH]ncc2ccc(Br)cc12]

7-Bromophthalazin-1(2H)-one (22a) In a 25 mL round bottom flask was dissolved 3,6-dibromoisobenzofuran-1(3H)-one (143 mg, 0.49 mmol) in ethanol (5 mL). Hydrazine monohydrate (0.12 mL, 2.48 mmol) was then added via a syringe and the solution was refluxed for 1.5 hours. The solution was cooled to room temperature and ice water (15 mL) was added to the reaction mixture. The precipitate was vacuum filtered and dried under a vacuum overnight to yield 22a as a white solid in 56% yield. ¹H NMR (500 MHz, DMSO-d₆) 6 12.82 (br. s., 1H), 8.39 (s, 1H), 8.30 (d, J=2.0 Hz, 1H), 8.11 (dd, J=8.5, 2.2 Hz, 1H), 7.90 (d, J=8.3 Hz, 1H). LCMS found 225.0 [M+H]¹.

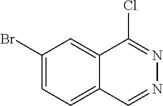 [SMILES: Clc1nncc2ccc(Br)cc12]

7-Bromo-1-chlorophthalazine (9a) In a flame dried 25 mL round bottom flask was added 7-bromophthalazin-1(2H)-one (205 mg, 0.91 mmol), anhydrous acetonitrile (9 mL), and phosphorus oxychloride (0.3 mL, 3.22 mmol). The mixture was refluxed for 2 hours, then cooled to 0° C., diluted with dichloromethane (20 mL), and quenched with a dropwise addition of sat. aq. NaHCO₃ (20 mL). The biphasic mixture was stirred vigorously and allowed to warm to room temperature. After 1 hour the layers were separated and the aqueous was extracted with dichloromethane (2×30 mL). The combined organic layers were washed with sat. aq. NaHCO₃ (25 mL), washed with brine (20 mL), dried over Na₂SO₄, and concentrated to yield 9a as an orange solid in 91% yield. ¹H NMR (500 MHz, CDCl₃) δ 9.45 (s, 1H), 8.49-8.51 (m, 1H), 8.10 (dd, J=8.8, 2.0 Hz, 1H), 7.91 (d, J=8.8 Hz, 1H). LCMS found 242.9 [M+H]⁺.

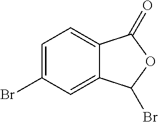 [SMILES: O=C1OC(Br)c2cc(Br)ccc21]

3,5-Dibromoisobenzofuran-1(3H)-one (21b) In a 25 mL round bottom flask was added 5-bromoisobenzofuran-1(3H)-one (499 mg, 2.34 mmol), N-bromosuccinimide (421 mg, 2.37 mmol), 2,2′-azobis(2-methylpropionitrile) (38 mg, 0.23 mmol), and chloroform (10 mL). The mixture was refluxed for 2.5 hours, then cooled to room temperature and quenched with sat. aq. NaHCO₃ (10 mL). The organic layer was removed, washed with water (10 mL), washed with brine (5 mL), and concentrated on to silica. The crude product was purified by flash column chromatography using 10% ethyl acetate in hexanes to yield 21b as a white solid in 49% yield.

¹H NMR (500 MHz, CDCl₃) δ 7.74-7.83 (m, 3H), 7.36 (s, 1H). LCMS does not ionize.

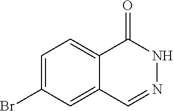 [SMILES: O=c1[nH]ncc2cc(Br)ccc12]

6-Bromophthalazin-1(2H)-one (22b) In a 25 mL round bottom flask was dissolved 3,5-dibromoisobenzofuran-1(3H)-one (302 mg, 1.04 mmol) in ethanol (10 mL). Hydrazine monohydrate (0.25 mL, 5.18 mmol) was then added via a syringe and the solution was refluxed for 1.5 hours. The solution was cooled to room temperature and ice water (30 mL) was added to the reaction mixture. The precipitate was vacuum filtered and dried under a vacuum overnight to yield 22b as a white solid in 73% yield. ¹H NMR (500 MHz, DMSO-d₆) δ 12.78 (br. s., 1H), 8.33 (s, 1H), 8.23 (d, J=2.0 Hz, 1H), 8.12 (d, J=8.3 Hz, 1H), 8.00 (dd, J=8.3, 2.0 Hz, 1H). LCMS found 224.9 [M+H]⁺.

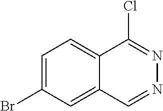 [SMILES: Clc1nncc2cc(Br)ccc12]

6-Bromo-1-chlorophthalazine (9c) In a flame dried 50 mL round bottom flask was added 6-bromophthalazin-1(2H)-one (402 mg, 1.78 mmol), anhydrous acetonitrile (18 mL), and phosphorus oxychloride (0.5 mL, 5.36 mmol). The mixture was refluxed for 2 hours, then cooled to 0° C., diluted with dichloromethane (40 mL), and quenched with a dropwise addition of sat. aq. NaHCO₃ (40 mL). The biphasic mixture was stirred vigorously and allowed to warm to room temperature. After 1 hour the layers were separated and the aqueous was extracted with dichloromethane (2×50 mL). The combined organic layers were washed with sat. aq. NaHCO₃ (40 mL), washed with brine (30 mL), dried over Na₂SO₄, and concentrated to yield 29c as a yellow solid in 94% yield. ¹H NMR (500 MHz, CDCl₃) δ 9.39 (s, 1H), 8.17-8.21 (m, 2H), 8.10 (dd, J=8.8, 2.0 Hz, 1H). LCMS found 242.9 [M+H]⁺.

General procedure A for the amination of 4-chloro-6-iodoquinoline-3-carbonitrile, and 4-chlorothienopyrimidines: To a solution of the appropriate aryl chloride (1 eq.) in 2-propanol (0.15M) was added 3-chloro-4-((3-fluorobenzypoxy)aniline or 4-(benzyloxy)-3-chloroaniline (1.1 eq.). The resulting mixture was refluxed overnight. The formed precipitate was collected by vacuum filtration to obtain the desired products.

General procedure B for the amination of 4-chloroquinolines and 1-chloroisoquinolines: To a solution of the appropriate aryl chloride (1 eq.) in 2-propanol (0.15M) was added 3-chloro-4-((3-fluorobenzyl)oxy)aniline or 4-(benzyloxy)-3-chloroaniline (1.1 eq.). The resulting mixture was refluxed overnight. The mixture was diluted with water, basified with 3M aq. NaOH to pH 12, and extracted with dichloromethane (3×). The combined organic layers were washed with water, washed with brine, dried over Na₂SO₄, and concentrated. The crude products were purified by flash column chromatography to obtain the desired products.

General Procedure C for the Amination of 4-Chlorocinnolines:

A solution of the appropriate 4-chlorocinnoline (1 eq.) and 3-chloro-4-((3-fluorobenzyl)oxy)aniline or 4-(benzyloxy)-3-chloroaniline (4 eq.) in toluene (0.1M) was refluxed for 2.5 hours and cooled to room temperature. Triethylamine (4 eq.) was added and the mixture was returned to reflux for an additional 30 minutes. The mixture was cooled back to room temperature and the formed yellow precipitate was vacuum filtered, washed with ethyl acetate, concentrated on to silica, and purified by flash column chromatography.

General Procedure D for the Amination of 1-Chlorophthalazines:

A solution of the appropriate 1-chlorophthalazine (1 eq.) and 3-chloro-4-((3-fluorobenzyl)oxy)aniline or 4-(benzyloxy)-3-chloroaniline (4 eq.) in anhydrous toluene (0.2M) was heated at 50° C. overnight. Water was added, the mixture was neutralized with 1M aq. NaOH, and was extracted with 5% methanol in dichloromethane. The combined organic layers were washed with brine, dried over Na₂SO₄, concentrated on to silica, and purified by flash column chromatography.

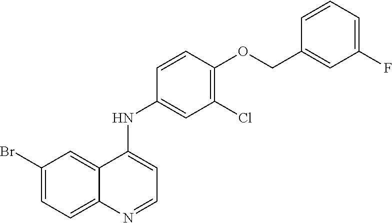 [SMILES: Fc1cccc(COc2ccc(Nc3ccnc4ccc(Br)cc34)cc2Cl)c1]

6-Bromo-N-(3-chloro-4-((3-fluorobenzyl)oxy)phenyl)quinolin-4-amine (23) Synthesized by general procedure B, FCC: 20-50% ethyl acetate in hexanes to yield 23 as a light brown solid in 90% yield. ¹H NMR (500 MHz, DMSO-d₆) δ 8.99 (s, 1H), 8.63 (s, 1H), 8.46 (d, J=5.4 Hz, 1H), 7.80 (d, J=1.5 Hz, 2H), 7.43-7.51 (m, 2H), 7.27-7.36 (m, 4H), 7.19 (td, J=8.7, 2.2 Hz, 1H), 6.80 (d, J=5.4 Hz, 1H), 5.26 (s, 2H). LCMS found 456.8 [M+H]⁺.

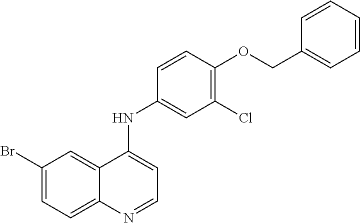 [SMILES: Clc1cc(Nc2ccnc3ccc(Br)cc23)ccc1OCc1ccccc1]

N-(4-(Benzyloxy)-3-chlorophenyl)-6-bromoquinolin-4-amine (24) Synthesized by general procedure B, FCC: 20-70% ethyl acetate in hexanes to yield 24 as a brown solid in 70% yield. ¹H NMR (500 MHz, DMSO-d₆) δ 8.98 (s, 1H), 8.63 (s, 1H), 8.45 (d, J=5.4 Hz, 1H), 7.80 (s, 2H), 7.50 (d, J=7.3 Hz, 2H), 7.39-7.46 (m, 3H), 7.35 (t, J=7.3 Hz, 1H), 7.30 (s, 2H), 6.79 (d, J=5.4 Hz, 1H), 5.23 (s, 2H). LCMS found 439.2 [M+H]⁺.

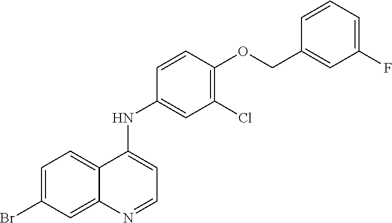 [SMILES: Fc1cccc(COc2ccc(Nc3ccnc4cc(Br)ccc34)cc2Cl)c1]

7-Bromo-N-(3-chloro-4-((3-fluorobenzyl)oxy)phenyl)quinolin-4-amine (25) Synthesized by general procedure B, FCC: 20-50% ethyl acetate in hexanes to yield 25 as a tan colored solid in 82% yield. ¹H NMR (399 MHz, DMSO-d₆) δ 9.05 (s, 1H), 8.43 (d, J=5.1 Hz, 1H), 8.31 (d, J=8.8 Hz, 1H), 8.05 (d, J=2.2 Hz, 1H), 7.67 (dd, J=8.8, 2.2 Hz, 1H), 7.41-7.52 (m, 2H), 7.25-7.37 (m, 4H), 7.18 (td, J=8.6, 2.6 Hz, 1H), 6.76 (d, J=5.9 Hz, 1H), 5.25 (s, 2H). LCMS found 456.8 [M+H]⁺.

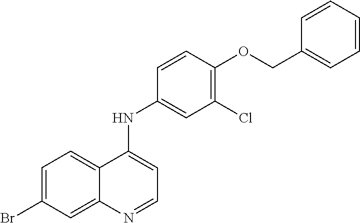 [SMILES: Clc1cc(Nc2ccnc3cc(Br)ccc23)ccc1OCc1ccccc1]

N-(4-(Benzyloxy)-3-chlorophenyl)-7-bromoquinolin-4-amine (26) Synthesized by general procedure B, FCC: 20-50% ethyl acetate in hexanes to yield 26 as an off-white solid in 83% yield. ¹H NMR (500 MHz, DMSO-d₆) δ 9.04 (s, 1H), 8.43 (d, J=4.9 Hz, 1H), 8.31 (d, J=9.3 Hz, 1H), 8.05 (d, J=2.0 Hz, 1H), 7.67 (dd, J=9.0, 2.2 Hz, 1H), 7.49 (d, J=6.8 Hz, 2H), 7.39-7.46 (m, 3H), 7.35 (t, J=7.3 Hz, 1H), 7.30 (s, 2H), 6.75 (d, J=5.4 Hz, 1H), 5.23 (s, 2H). LCMS found 439.2 [M+H]⁺.

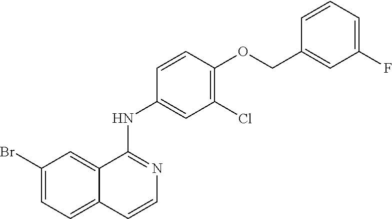 [SMILES: Fc1cccc(COc2ccc(Nc3nccc4ccc(Br)cc34)cc2Cl)c1]

7-Bromo-N-(3-chloro-4-((3-fluorobenzyl)oxy)phenyl)isoquinolin-1-amine (27) Synthesized by general procedure B, FCC: 10-30% ethyl acetate in hexanes to yield 27 as a pale red-brown solid in 97% yield. ¹H NMR (500 MHz, DMSO-d₆) δ 9.24 (s, 1H), 8.80 (s, 1H), 8.07 (d, J=2.4 Hz, 1H), 8.02 (d, J=5.9 Hz, 1H), 7.82 (dd, J=8.8, 1.5 Hz, 1H), 7.72-7.79 (m, 2H), 7.41-7.49 (m, 1H), 7.27-7.35 (m, 2H), 7.13-7.23 (m, 3H), 5.21 (s, 2H). LCMS found 456.8 [M+H]⁺.

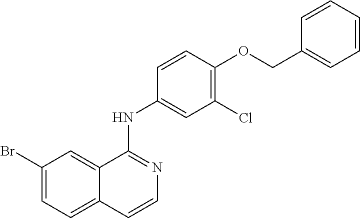 [SMILES: Clc1cc(Nc2nccc3ccc(Br)cc23)ccc1OCc1ccccc1]

N-(4-(Benzyloxy)-3-chlorophenyl)-7-bromoisoquinolin-1-amine (28) Synthesized by general procedure B, FCC: 10-30% ethyl acetate in hexanes to yield 28 as a light red solid in 93% yield. ¹H NMR (500 MHz, DMSO-d₆) δ 9.22 (s, 1H), 8.80 (s, 1H), 8.07 (d, J=2.4 Hz, 1H), 8.02 (d, J=5.9 Hz, 1H), 7.81 (dd, J=8.8, 1.5 Hz, 1H), 7.73-7.78 (m, 2H), 7.48 (d, J=7.3 Hz, 2H), 7.40 (t, J=7.6 Hz, 2H), 7.33 (t, J=7.3 Hz, 1H), 7.21 (d, J=8.8 Hz, 1H), 7.15 (d, J=5.9 Hz, 1H), 5.18 (s, 2H). LCMS found 439.2 [M+H]⁺.

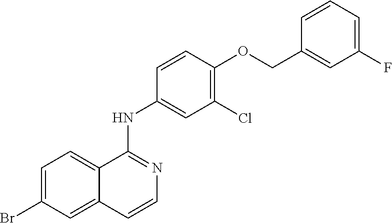 [SMILES: Fc1cccc(COc2ccc(Nc3nccc4cc(Br)ccc34)cc2Cl)c1]

6-Bromo-N-(3-chloro-4-((3-fluorobenzyl)oxy)phenyl)isoquinolin-1-amine (29) Synthesized by general procedure B, FCC: 10-30% ethyl acetate in hexanes to yield 29 as a salmon colored solid in 91% yield. ¹H NMR (500 MHz, DMSO-d₆) δ 9.27 (s, 1H), 8.45 (d, J=8.8 Hz, 1H), 8.08 (dd, J=12.0, 2.2 Hz, 2H), 8.01 (d, J=5.4 Hz, 1H), 7.75 (ddd, J=13.8, 9.2, 2.4 Hz, 2H), 7.46 (m, J=5.9 Hz, 1H), 7.27-7.35 (m, 2H), 7.10-7.23 (m, 3H), 5.22 (s, 2H). LCMS found 456.8 [M+H]⁺.

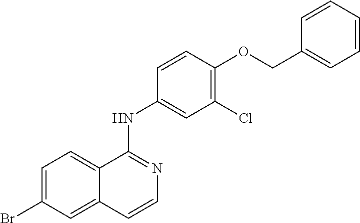 [SMILES: Clc1cc(Nc2nccc3cc(Br)ccc23)ccc1OCc1ccccc1]

N-(4-(Benzyloxy)-3-chlorophenyl)-6-bromoisoquinolin-1-amine (30) Synthesized by general procedure B, FCC: 10-30% ethyl acetate in hexanes to yield 30 as a burnt orange solid in 81% yield. ¹H NMR (500 MHz, DMSO-d₆) δ 9.24 (s, 1H), 8.45 (d, J=8.8 Hz, 1H), 8.10 (d, J=2.0 Hz, 1H), 8.05 (d, J=2.4 Hz, 1H), 8.01 (d, J=5.9 Hz, 1H), 7.76 (dd, J=9.0, 2.2 Hz, 1H), 7.72 (dd, J=8.8, 2.4 Hz, 1H), 7.48 (d, J=6.8 Hz, 2H), 7.41 (t, J=7.3 Hz, 2H), 7.34 (t, J=7.8 Hz, 1H), 7.21 (d, J=9.3 Hz, 1H), 7.13 (d, J=5.9 Hz, 1H), 5.19 (s, 2H). LCMS found 439.2 [M+H]⁺.

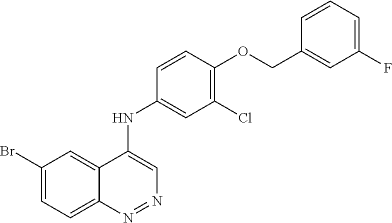 [SMILES: Fc1cccc(COc2ccc(Nc3cnnc4ccc(Br)cc34)cc2Cl)c1]

6-Bromo-N-(3-chloro-4-((3-fluorobenzyl)oxy)phenyl)cinnolin-4-amine (31) Synthesized by general procedure C, FCC: 5% methanol in dichloromethane to yield an inseparable 10:1 mixture of 31 and dichloro side product as a vibrant yellow solid in 54% yield. ¹H NMR (500 MHz, DMSO-d₆) δ 9.33 (s, 1H), 8.78 (s, 1H), 8.70 (d, J=1.5 Hz, 1H), 8.14 (d, J=8.8 Hz, 1H), 7.97 (dd, J=9.3, 1.5 Hz, 1H), 7.54 (d, J=2.0 Hz, 1H), 7.48 (td, J=8.3, 6.3 Hz, 1H), 7.40 (dd, J=8.8, 2.4 Hz, 1H), 7.30-7.37 (m, 3H), 7.20 (td, J=9.3, 2.0 Hz, 1H), 5.29 (s, 2H). LCMS found 458.0/414.0 [M+H]⁺.

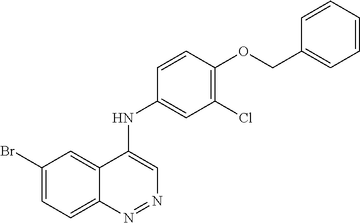 [SMILES: Clc1cc(Nc2cnnc3ccc(Br)cc23)ccc1OCc1ccccc1]

N-(4-(Benzyloxy)-3-chlorophenyl)-6-bromocinnolin-4-amine (32) Synthesized by general procedure C, FCC: 5% methanol in dichloromethane to yield an inseparable 10:1 mixture of 32 and dichloro side product as a yellow solid in 68% yield. ¹H NMR (500 MHz, DMSO-d₆) δ 9.32 (s, 1H), 8.78 (s, 1H), 8.71 (d, J=2.0 Hz, 1H), 8.14 (d, J=9.3 Hz, 1H), 7.97 (dd, J=9.0, 1.7 Hz, 1H), 7.47-7.55 (m, 3H), 7.44 (t, J=7.6 Hz, 2H), 7.33-7.41 (m, 3H), 5.26 (s, 2H). LCMS found 439.9/396.0 [M+H]⁺.

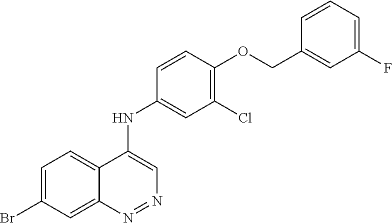 [SMILES: Fc1cccc(COc2ccc(Nc3cnnc4cc(Br)ccc34)cc2Cl)c1]

7-Bromo-N-(3-chloro-4-((3-fluorobenzyl)oxy)phenyl)cinnolin-4-amine (33) Synthesized by general procedure C, FCC: 5% methanol in dichloromethane to yield 33 as a brown solid in 55% yield with 84% purity. ¹H NMR (500 MHz, DMSO-d₆) δ 9.43 (s, 1H), 8.76 (s, 1H), 8.41 (d, J=2.0 Hz, 1H), 8.35 (d, J=9.3 Hz, 1H), 7.90 (dd, J=9.0, 2.2 Hz, 1H), 7.55 (d, J=2.4 Hz, 1H), 7.49 (m, J=6.3 Hz, 1H), 7.41 (dd, J=8.8, 2.4 Hz, 1H), 7.31-7.37 (m, 3H), 7.20 (td, J=8.4, 2.7 Hz, 1H), 5.29 (s, 2H). LCMS found 457.9 [M+H]⁺.

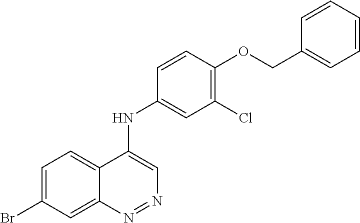 [SMILES: Clc1cc(Nc2cnnc3cc(Br)ccc23)ccc1OCc1ccccc1]

N-(4-(Benzyloxy)-3-chlorophenyl)-7-bromocinnolin-4-amine (34) Synthesized by general procedure C, FCC: 5% methanol in dichloromethane to yield 34 as a metallic bronze colored solid in 57% yield with 85% purity. ¹H NMR (500 MHz, DMSO-d₆) δ 9.42 (s, 1H), 8.75 (s, 1H), 8.41 (d, J=2.0 Hz, 1H), 8.35 (d, J=8.8 Hz, 1H), 7.90 (dd, J=9.0, 2.2 Hz, 1H), 7.54 (d, J=2.4 Hz, 1H), 7.51 (d, J=7.3 Hz, 2H), 7.43 (t, J=7.6 Hz, 2H), 7.40 (dd, J=8.8, 2.4 Hz, 1H), 7.33-7.38 (m, 2H), 5.26 (s, 2H). LCMS found 440.0 [M+H]⁺.

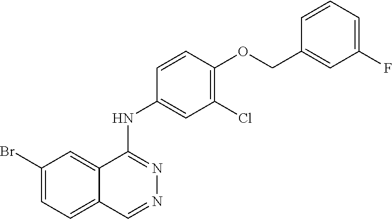 [SMILES: Fc1cccc(COc2ccc(Nc3nncc4ccc(Br)cc34)cc2Cl)c1]

7-Bromo-N-(3-chloro-4-((3-fluorobenzyl)oxy)phenyl)phthalazin-1-amine (35) Synthesized by general procedure D, FCC: 5-20% ethyl acetate in dichloromethane to yield 35 as a yellow solid in 69% yield. ¹H NMR (500 MHz, DMSO-d₆) δ 9.21 (s, 1H), 9.13 (s, 1H), 8.87 (s, 1H), 8.16 (d, J=2.4 Hz, 1H), 8.11 (dd, J=8.8, 1.5 Hz, 1H), 7.99 (d, J=8.8 Hz, 1H), 7.81 (dd, J=8.8, 2.4 Hz, 1H), 7.47 (td, J=7.9, 6.1 Hz, 1H), 7.29-7.36 (m, 2H), 7.25 (d, J=9.3 Hz, 1H), 7.17 (td, J=8.7, 2.2 Hz, 1H), 5.24 (s, 2H). LCMS found 457.9 [M+H]⁺.

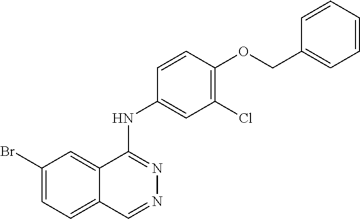 [SMILES: Clc1cc(Nc2nncc3ccc(Br)cc23)ccc1OCc1ccccc1]

N-(4-(Benzyloxy)-3-chlorophenyl)-7-bromophthalazin-1-amine (36) Synthesized by general procedure D, FCC: 5-20% ethyl acetate in dichloromethane to yield 36 as a light greenish brown solid in 78% yield. ¹H NMR (500 MHz, DMSO-d₆) δ 9.20 (s, 1H), 9.14 (s, 1H), 8.88 (s, 1H), 8.15 (d, J=2.4 Hz, 1H), 8.13 (dd, J=8.5, 1.7 Hz, 1H), 8.00 (d, J=8.8 Hz, 1H), 7.80 (dd, J=9.0, 2.7 Hz, 1H), 7.50 (d, J=7.3 Hz, 2H), 7.42 (t, J=7.6 Hz, 2H), 7.35 (t, J=7.3 Hz, 1H), 7.27 (d, J=8.8 Hz, 1H), 5.21 (s, 2H). LCMS found 440.0 [M+H]⁺.

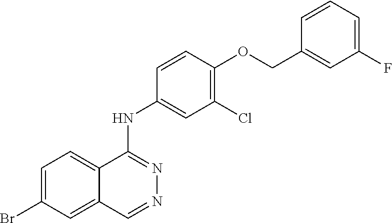 [SMILES: Fc1cccc(COc2ccc(Nc3nncc4cc(Br)ccc34)cc2Cl)c1]

6-Bromo-N-(3-chloro-4-((3-fluorobenzyl)oxy)phenyl)phthalazin-1-amine (37) Synthesized by general procedure D, FCC: 5-20% ethyl acetate in dichloromethane to yield 37 as a light brown solid in 64% yield. ¹H NMR (500 MHz, DMSO-d₆) δ 9.42 (s, 1H), 9.09 (s, 1H), 8.62 (d, J=9.3 Hz, 1H), 8.33 (d, J=2.0 Hz, 1H), 8.19 (d, J=2.4 Hz, 1H), 8.16 (dd, J=9.0, 2.2 Hz, 1H), 7.83 (dd, J=8.8, 2.4 Hz, 1H), 7.47 (td, J=7.8, 5.9 Hz, 1H), 7.32 (m, J=7.3 Hz, 2H), 7.25 (d, J=9.3 Hz, 1H), 7.17 (td, J=8.5, 2.0 Hz, 1H), 5.24 (s, 2H). LCMS found 458.0 [M+H]⁺.

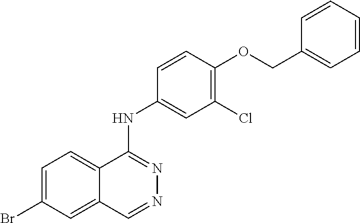 [SMILES: Clc1cc(Nc2nncc3cc(Br)ccc23)ccc1OCc1ccccc1]

N-(4-(Benzyloxy)-3-chlorophenyl)-6-bromophthalazin-1-amine (38) Synthesized by general procedure D, FCC: 5-30% ethyl acetate in dichloromethane to yield 38 as a green-gray colored solid in 75% yield. ¹H NMR (500 MHz, DMSO-d₆) δ 9.25 (s, 1H), 9.09 (s, 1H), 8.51 (d, J=8.8 Hz, 1H), 8.33 (d, J=2.0 Hz, 1H), 8.17 (dd, J=8.8, 2.0 Hz, 1H), 8.14 (d, J=2.9 Hz, 1H), 7.78 (dd, J=9.0, 2.7 Hz, 1H), 7.49 (d, J=6.8 Hz, 2H), 7.42 (t, J=7.6 Hz, 2H), 7.35 (t, J=7.3 Hz, 1H), 7.26 (d, J=8.8 Hz, 1H), 5.21 (s, 2H). LCMS found 440.0 [M+H]⁺.

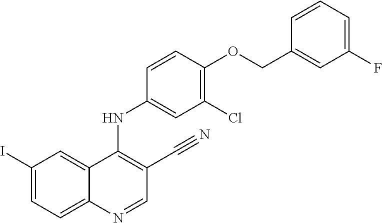 [SMILES: N#Cc1cnc2ccc(I)cc2c1Nc1ccc(OCc2cccc(F)c2)c(Cl)c1]

4-((3-Chloro-4-((3-fluorobenzyl)oxy)phenyl)amino)-6-iodoquinoline-3-carbonitrile (39) Synthesized by general procedure A, collected as a yellow solid in 80% yield. ¹H NMR (400 MHz, DMSO-d₆) δ 9.03 (s, 1H), 8.85 (br, s, 1H), 8.23 (d, J=8.8 Hz, 1H), 7.73 (d, J=8.8, 1H), 7.59 (d, J=2.4, 1H), 7.45-7.50 (m, 1H), 7.30-7.39 (m, 4H), 7.18-7.22 (m, 1H), 5.30 (s, 2H). LCMS found 530.7 [M+H]⁺.

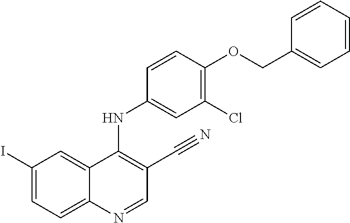 [SMILES: N#Cc1cnc2ccc(I)cc2c1Nc1ccc(OCc2ccccc2)c(Cl)c1]

4-((4-(Benzyloxy)-3-chlorophenyl)amino)-6-iodoquinoline-3-carbonitrile (40) Synthesized by general procedure A, collected as a yellow solid in 52% yield. ¹H NMR (500 MHz, DMSO-d₆) δ ppm 9.02 (s, 1H), 8.82 (br, s, 1H), 8.22 (d, J=9 Hz, 1H), 7.72 (d, J=8.5 Hz, 1H), 7.57 (s, 1H), 7.5 (d, J=7 Hz, 2H), 7.43 (t, J=7.5 Hz, 2H), 7.35 (m, 3H), 5.26 (s, 2H). LCMS found 512.7 [M+H]⁺.

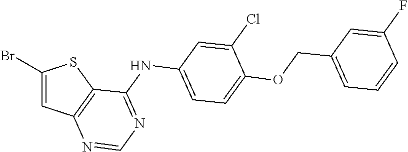 [SMILES: Fc1cccc(COc2ccc(Nc3ncnc4cc(Br)sc34)cc2Cl)c1]

6-bromo-N-(3-chloro-4-((3-fluorobenzyl)oxy)phenyl)thieno [3,2-d]pyrimidin-4-amine (41). Synthesized by general procedure A in 88% yield. ¹H NMR (400 MHz, DMSO-d6) δ ppm 5.28 (s, 2H), 7.19 (td, J=8.1, 2.2 Hz, 1H), 7.31 (m, 3H), 7.47 (m, 1H), 7.57 (dd, J=8.8, 2.2 Hz, 1H), 7.74 (s, 1H), 7.87 (d, J=2.9 Hz, 1H), 8.71 (s, 1H), 10.64 (s, 1H). LCMS found 463.9, [M+H]⁺.

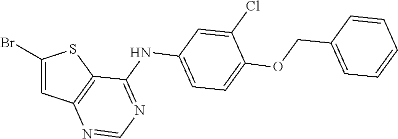 [SMILES: Clc1cc(Nc2ncnc3cc(Br)sc23)ccc1OCc1ccccc1]

N-(4-(benzyloxy)-3-chlorophenyl)-6-bromothieno[3,2-d]pyrimidin-4-amine (42). Synthesized by general procedure A in 74% yield. ¹H NMR (500 MHz, DMSO-d6) δ ppm 5.23 (s, 2H), 7.26 (d, J=8.8 Hz, 1H), 7.35 (t, J=7.3 Hz, 1H), 7.42 (t, J=7.6 Hz, 2H), 7.49 (d, J=7.8 Hz, 2H), 7.58 (dd, J=8.8, 2.9 Hz, 1H), 7.68 (s, 1H), 7.89 (d, J=2.0 Hz, 1H), 8.54 (s, 1H), 9.71 (s, 1H). LCMS found 445.9, [M+H]⁺.

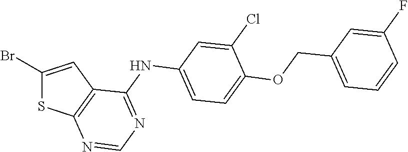 [SMILES: Fc1cccc(COc2ccc(Nc3ncnc4sc(Br)cc34)cc2Cl)c1]

6-bromo-N-(3-chloro-4-((3-fluorobenzyl)oxy)phenyl)thieno[2,3-d]pyrimidin-4-amine (43). Synthesized by general procedure A in 88% yield. ¹H NMR (500 MHz, DMSO-d₆) δ 9.80 (s, 1H), 8.49 (s, 1H), 8.12 (s, 1H), 8.02 (d, J=2.44 Hz, 1H), 7.67 (dd, J=2.69, 9.03 Hz, 1H), 7.43-7.49 (m, 1H), 7.24-7.35 (m, 3H), 7.18 (dt, J=2.44, 8.55 Hz, 1H), 5.24 (s, 2H). ¹H NMR LCMS found 463.8, [M+H]⁺.

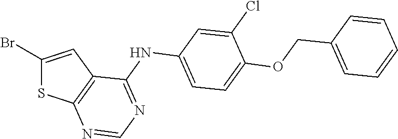 [SMILES: Clc1cc(Nc2ncnc3sc(Br)cc23)ccc1OCc1ccccc1]

N-(4-(benzyloxy)-3-chlorophenyl)-6-bromothieno [2,3-d]pyrimidin-4-amine (44). Synthesized by general procedure A in 63% yield. ¹H NMR (500 MHz, DMSO-d6) δ ppm 5.21 (s, 2H), 7.27 (d, J=9.3 Hz, 1H), 7.34 (m, 1H), 7.41 (m, 2H), 7.49 (m, 2H), 7.68 (dd, J=8.8, 2.4 Hz, 1H), 8.02 (d, J=2.4 Hz, 1H), 8.19 (s, 1H), 8.49 (s, 1H), 9.94 (s, 1H). LCMS found 445.9, [M+H]⁺.

### General Procedure A for Suzuki Couplings:

In a 2-5 mL microwave vial equipped with a stir bar was added aryl bromide (1 eq.), boronic ester (1.3 eq.), 1:1 water/ethanol (0.04M), triethylamine (3 eq.), and palladium(II) acetate (0.01M in acetone, 1 mol %). The vial was sealed with a septum and the contents were irradiated to and held at 120° C. with stirring for 1 hour. The reaction mixture was cooled to room temperature, diluted with water (8 mL), and extracted with dichloromethane (3×8 mL). The combined organic layers were washed with aq. NaOH (1M, 2×5 mL), water (5 mL), and brine (5 mL). The organic layer was then dried over Na₂SO₄ and concentrated on to silica. The crude product was purified by flash column chromatography.

### General Procedure B for Suzuki Couplings:

In a 2-5 mL microwave vial equipped with a stir bar was added aryl bromide (1 eq.), boronic ester (1.3 eq.), 1:1 water/ethanol (0.04M), triethylamine (3 eq.), and bis(triphenylphosphine)palladium(II) chloride (2.5 mol %). The vial was sealed with a septum and the contents were irradiated to and held at 120° C. with stirring for 1 hour. The reaction mixture was cooled to room temperature, diluted with water (8 mL), and extracted with dichloromethane (3×8 mL). The combined organic layers were washed with aq. NaOH (1M, 2×5 mL), water (5 mL), and brine (5 mL). The organic layer was then dried over Na₂SO₄ and concentrated on to silica. The crude product was purified by flash column chromatography.

### General Procedure C for Suzuki Couplings:

In a 2-5 mL microwave vial equipped with a stir bar was added aryl bromide (1 eq.), boronic ester (1.3 eq.), 1:1 water/ethanol (0.04M), triethylamine (3 eq.), and palladium(II) acetate (5 mol %). The vial was sealed with a septum and the contents were irradiated to and held at 120° C. with stirring for 3 hours. The reaction mixture was cooled to room temperature, diluted with water (8 mL), and extracted with dichloromethane (3×8 mL). The combined organic layers were washed with aq. NaOH (1M, 2×5 mL), water (5 mL), and brine (5 mL). The organic layer was then dried over Na₂SO₄ and concentrated on to silica. The crude product was purified by flash column chromatography.

### General Procedure D for Suzuki Couplings:

To a solution of the appropriate aryl iodide (1 eq.) in 3:2 dimethoxyethane/ethanol (0.05M) was added the appropriate aryl boronic ester (1.1 eq.), aq. 2M Na₂CO₃ (6 eq.), and Pd(PPh₃)₄ (5 mol %). The mixture was purged with nitrogen and heated at 85° C. for 7 hours. The mixture was cooled to room temperature, filtered, and the filtrate concentrated. The residue was dissolved in ethyl acetate, washed with water, washed with brine, dried over Na₂SO₄, and purified by flash column chromatography.

### General Procedure E for Suzuki Couplings:

To a solution of the appropriate aryl bromide (1 eq.) in 3:2 dimethoxyethane/ethanol (0.05M) was added the appropriate aryl boronic ester (1.2 eq.), aq. 2M Na₂CO₃ (6 eq.), and Pd(PPh₃)₄ (7 mol %). The mixture was heated at 85° C. for 12 hours, then cooled to room temperature and the solvents removed under reduced pressure. The residue was purified by silica column chromatography (hexanes/ethyl acetate) then by reverse phase chromatography (water/acetonitrile) unless otherwise mentioned.

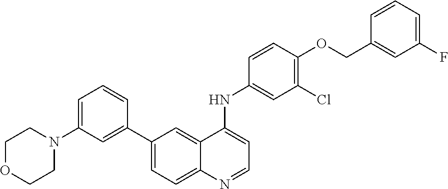 [SMILES: Fc1cccc(COc2ccc(Nc3ccnc4ccc(-c5cccc(N6CCOCC6)c5)cc34)cc2Cl)c1]

N-(3-Chloro-4-((3-fluorobenzyl)oxy)phenyl)-6-(3-morpholinophenyl)quinolin-4-amine (45) General procedure A, FCC: 2-5% methanol in dichloromethane, isolated as a yellow solid in 27% yield. ¹H NMR (500 MHz, DMSO-d₆) δ ppm 9.10 (br. s., 1H), 8.61 (d, J=2.0 Hz, 1H), 8.43 (d, J=5.4 Hz, 1H), 8.04 (dd, J=8.8, 2.0 Hz, 1H), 7.92 (d, J=8.8 Hz, 1H), 7.45-7.52 (m, 2H), 7.39 (t, J=7.8 Hz, 1H), 7.29-7.37 (m, 6H), 7.20 (td, J=8.8, 2.0 Hz, 1H), 7.00 (dd, J=8.1, 1.7 Hz, 1H), 6.77 (d, J=5.4 Hz, 1H), 5.28 (s, 2H), 3.75-3.81 (m, 4H), 3.19-3.26 (m, 4H). LCMS found 540.1 [M+H]⁺.

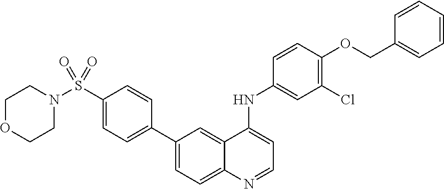 [SMILES: O=S(=O)(c1ccc(-c2ccc3nccc(Nc4ccc(OCc5ccccc5)c(Cl)c4)c3c2)cc1)N1CCOCC1]

N-(4-(Benzyloxy)-3-chlorophenyl)-6-(4-(morpholinosulfonyl)phenyl)quinolin-4-amine (47) General procedure A, FCC: 0-5% methanol in dichloromethane, isolated as a tan colored solid in 65% yield. ¹H NMR (500 MHz, DMSO-d₆) δ ppm 9.14 (s, 1H), 8.78 (d, J=2.0 Hz, 1H), 8.47 (d, J=5.4 Hz, 1H), 8.17 (d, J=8.8 Hz, 2H), 8.11 (dd, J=8.8, 2.0 Hz, 1H), 7.98 (d, J=8.8 Hz, 1H), 7.88 (d, J=8.3 Hz, 2H), 7.51 (d, J=7.3 Hz, 2H), 7.48 (d, J=1.5 Hz, 1H), 7.43 (t, J=7.6 Hz, 2H), 7.32-7.39 (m, 3H), 6.80 (d, J=5.4 Hz, 1H), 5.25 (s, 2H), 3.61-3.70 (m, 4H), 2.88-2.97 (m, 4H). LCMS found 586.1 [M+H]⁺.

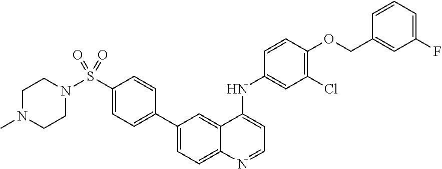 [SMILES: CN1CCN(S(=O)(=O)c2ccc(-c3ccc4nccc(Nc5ccc(OCc6cccc(F)c6)c(Cl)c5)c4c3)cc2)CC1]

N-(3-Chloro-4-((3-fluorobenzyl)oxy)phenyl)-6-(4-((4-methylpiperazin-1-yl)sulfonyl)phenyl)quinolin-4-amine (49) General procedure A, FCC: 5-10% methanol in dichloromethane, isolated as a yellow solid in 40% yield. ¹H NMR (500 MHz, DMSO-d₆) δ ppm 9.19 (br. s., 1H), 8.78 (d, J=2.0 Hz, 1H), 8.47 (d, J=5.4 Hz, 1H), 8.16 (d, J=8.8 Hz, 2H), 8.12 (dd, J=8.8, 2.0 Hz, 1H), 7.98 (d, J=8.8 Hz, 1H), 7.87 (d, J=8.8 Hz, 2H), 7.45-7.52 (m, 2H), 7.30-7.38 (m, 4H), 7.20 (td, J=8.7, 2.7 Hz, 1H), 6.81 (d, J=5.4 Hz, 1H), 5.28 (s, 2H), 2.95 (br. s., 4H), 2.35-2.43 (m, 4H), 2.15 (s, 3H). LCMS found 617.1 [M+H]⁺.

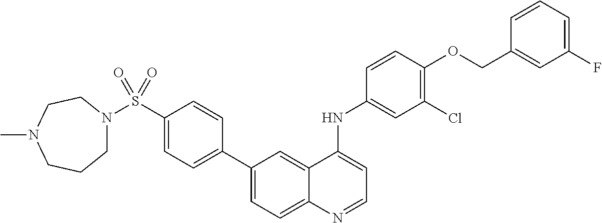 [SMILES: CN1CCCN(S(=O)(=O)c2ccc(-c3ccc4nccc(Nc5ccc(OCc6cccc(F)c6)c(Cl)c5)c4c3)cc2)CC1]

N-(3-Chloro-4-((3-fluorobenzyl)oxy)phenyl)-6-(4-((4-methyl-1,4-diazepan-1-yl)sulfonyl)phenyl)quinolin-4-amine (51) General procedure A, FCC: 5-20% methanol in dichloromethane, then prep HPLC: 5-95% acetonitrile in water, isolated as a yellow solid in 10% yield. ¹H NMR (500 MHz, DMSO-d₆) δ ppm 8.77 (d, J=2.0 Hz, 1H), 8.46 (d, J=5.4 Hz, 1H), 8.27 (s, 1H), 8.07-8.15 (m, 3H), 7.97 (d, J=8.8 Hz, 1H), 7.92 (d, J=8.3 Hz, 2H), 7.45-7.52 (m, 2H), 7.30-7.38 (m, 4H), 7.20 (td, J=8.5, 2.4 Hz, 1H), 6.80 (d, J=5.4 Hz, 1H), 5.28 (s, 2H), 3.36 (m, J=5.2, 2.5, 2.5 Hz, 2H), 3.33 (t, J=6.3 Hz, 2H), 2.53-2.57 (m, 2H), 2.47 (m, J=5.9 Hz, 2H), 2.22 (s, 3H), 1.74 (quin, J=5.9 Hz, 2H). LCMS found 631.2 [M+H]⁺.

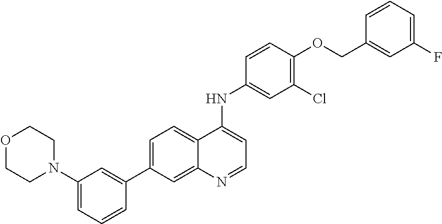 [SMILES: Fc1cccc(COc2ccc(Nc3ccnc4cc(-c5cccc(N6CCOCC6)c5)ccc34)cc2Cl)c1]

N-(3-Chloro-4-((3-fluorobenzyl)oxy)phenyl)-7-(3-morpholinophenyl)quinolin-4-amine (46) General procedure A, FCC: 2-5% methanol in dichloromethane, isolated as a yellow solid in 24% yield. ¹H NMR (500 MHz, DMSO-d₆) δ ppm 8.99 (br. s., 1H), 8.47 (d, J=5.4 Hz, 1H), 8.41 (d, J=8.8 Hz, 1H), 8.12 (d, J=2.0 Hz, 1H), 7.88 (dd, J=8.8, 1.5 Hz, 1H), 7.45-7.51 (m, 2H), 7.26-7.40 (m, 7H), 7.20 (td, J=8.8, 2.4 Hz, 1H), 7.02 (dd, J=8.3, 2.0 Hz, 1H), 6.76 (d, J=5.4 Hz, 1H), 5.27 (s, 2H), 3.74-3.82 (m, 4H), 3.20-3.27 (m, 4H). LCMS found 540.2 [M+H]⁺.

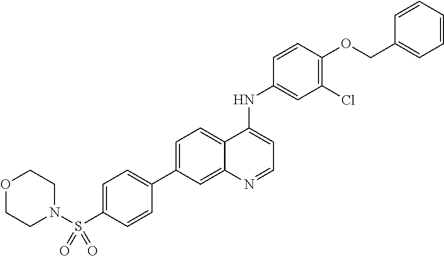 [SMILES: O=S(=O)(c1ccc(-c2ccc3c(Nc4ccc(OCc5ccccc5)c(Cl)c4)ccnc3c2)cc1)N1CCOCC1]

N-(4-(Benzyloxy)-3-chlorophenyl)-7-(4-(morpholinosulfonyl)phenyl)quinolin-4-amine (48) General procedure A, FCC: 2-5% methanol in dichloromethane, isolated as a tan colored solid in 63% yield. ¹H NMR (500 MHz, DMSO-d₆) δ ppm 9.02 (s, 1H), 8.50 (m, J=5.4 Hz, 2H), 8.25 (d, J=1.5 Hz, 1H), 8.18 (d, J=8.8 Hz, 2H), 7.97 (dd, J=8.8, 1.5 Hz, 1H), 7.87 (d, J=8.3 Hz, 2H), 7.51 (d, J=7.3 Hz, 2H), 7.47 (d, J=2.0 Hz, 1H), 7.44 (t, J=7.6 Hz, 2H), 7.36 (m, J=6.8 Hz, 1H), 7.30-7.34 (m, 2H), 6.79 (d, J=5.4 Hz, 1H), 5.24 (s, 2H), 3.62-3.70 (m, 4H), 2.90-2.97 (m, 4H). LCMS found 586.2 [M+H]⁺.

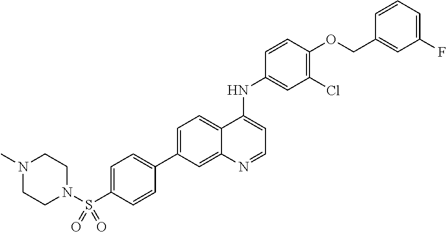 [SMILES: CN1CCN(S(=O)(=O)c2ccc(-c3ccc4c(Nc5ccc(OCc6cccc(F)c6)c(Cl)c5)ccnc4c3)cc2)CC1]

N-(3-Chloro-4-((3-fluorobenzyl)oxy)phenyl)-7-(4-((4-methylpiperazin-1-yl)sulfonyl)phenyl)quinolin-4-amine (50) General procedure A, FCC: 5-10% methanol in dichloromethane, isolated as a yellow solid in 33% yield. ¹H NMR (500 MHz, DMSO-d₆) δ ppm 9.10 (br. s., 1H), 8.50 (d, J=8.3 Hz, 2H), 8.25 (d, J=2.0 Hz, 1H), 8.16 (d, J=8.3 Hz, 2H), 7.97 (dd, J=8.8, 2.0 Hz, 1H), 7.87 (d, J=8.3 Hz, 2H), 7.45-7.52 (m, 2H), 7.29-7.38 (m, 4H), 7.20 (td, J=8.5, 2.0 Hz, 1H), 6.80 (d, J=5.4 Hz, 1H), 5.28 (s, 2H), 2.96 (br. s., 4H), 2.40 (br. s., 4H), 2.16 (s, 3H). LCMS found 617.1 [M+H]⁺.

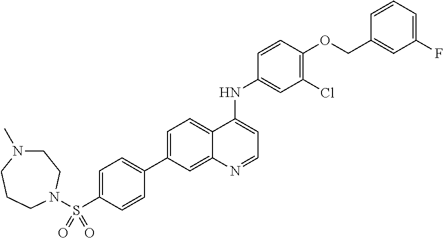 [SMILES: CN1CCCN(S(=O)(=O)c2ccc(-c3ccc4c(Nc5ccc(OCc6cccc(F)c6)c(Cl)c5)ccnc4c3)cc2)CC1]

N-(3-Chloro-4-((3-fluorobenzyl)oxy)phenyl)-7-(4-((4-methyl-1,4-diazepan-1-yl)sulfonyl)phenyl)quinolin-4-amine (52) General procedure A, FCC: 5-20% methanol in dichloromethane, isolated as a brown solid in 32% yield. ¹H NMR (500 MHz, DMSO-d₆) δ ppm 9.06 (s, 1H), 8.47-8.52 (m, 2H), 8.23 (d, J=2.0 Hz, 1H), 8.12 (d, J=8.3 Hz, 2H), 7.96 (dd, J=8.8, 2.0 Hz, 1H), 7.91 (d, J=8.3 Hz, 2H), 7.45-7.52 (m, 2H), 7.29-7.37 (m, 4H), 7.20 (td, J=8.5, 2.4 Hz, 1H), 6.79 (d, J=4.9 Hz, 1H), 5.27 (s, 2H), 3.39 (m, J=3.4 Hz, 2H), 3.31-3.35 (m, 2H), 2.63 (br. s., 2H), 2.58 (br. s., 2H), 2.29 (br. s., 3H), 1.74-1.83 (m, 2H). LCMS found 631.2 [M+H]⁺.

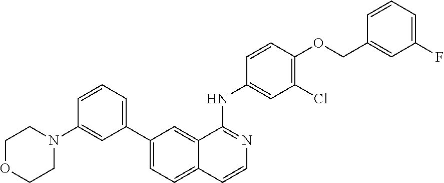 [SMILES: Fc1cccc(COc2ccc(Nc3nccc4ccc(-c5cccc(N6CCOCC6)c5)cc34)cc2Cl)c1]

N-(3-Chloro-4-((3-fluorobenzyl)oxy)phenyl)-7-(3-morpholinophenyl)isoquinolin-1-amine (53) General procedure A, FCC: 30-50% ethyl acetate in hexanes, isolated as a biege solid in 59% yield. ¹H NMR (500 MHz, DMSO-d₆) δ ppm 9.27 (s, 1H), 8.71 (s, 1H), 8.06 (d, J=2.4 Hz, 1H), 8.03 (dd, J=8.3, 1.5 Hz, 1H), 7.98 (d, J=5.4 Hz, 1H), 7.88 (d, J=8.8 Hz, 1H), 7.76 (dd, J=8.8, 2.4 Hz, 1H), 7.47 (td, J=8.1, 6.3 Hz, 1H), 7.40 (t, J=7.8 Hz, 1H), 7.29-7.36 (m, 4H), 7.23 (d, J=9.3 Hz, 1H), 7.15-7.21 (m, 2H), 7.02 (dd, J=8.3, 2.0 Hz, 1H), 5.23 (s, 2H), 3.75-3.82 (m, 4H), 3.20-3.26 (m, 4H). LCMS found 540.2 [M+H]⁺.

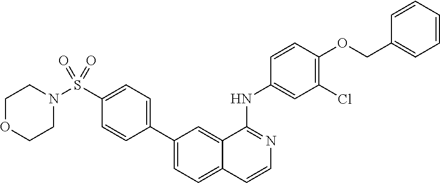 [SMILES: O=S(=O)(c1ccc(-c2ccc3ccnc(Nc4ccc(OCc5ccccc5)c(Cl)c4)c3c2)cc1)N1CCOCC1]

N-(4-(Benzyloxy)-3-chlorophenyl)-7-(4-(morpholinosulfonyl)phenyl)isoquinolin-1-amine (55) General procedure A, FCC: 20-50% ethyl acetate in hexanes, isolated as an orange solid in 55% yield. ¹H NMR (500 MHz, DMSO-d₆) δ ppm 9.35 (s, 1H), 8.86 (s, 1H), 8.18 (d, J=8.8 Hz, 2H), 8.12 (dd, J=8.8, 1.5 Hz, 1H), 8.04 (d, J=2.4 Hz, 1H), 8.03 (d, J=5.9 Hz, 1H), 7.96 (d, J=8.3 Hz, 1H), 7.90 (d, J=8.3 Hz, 2H), 7.76 (dd, J=9.3, 2.9 Hz, 1H), 7.50 (d, J=6.8 Hz, 2H), 7.42 (t, J=7.3 Hz, 2H), 7.35 (t, J=7.3 Hz, 1H), 7.25 (d, J=9.3 Hz, 1H), 7.22 (d, J=5.9 Hz, 1H), 5.21 (s, 2H), 3.63-3.70 (m, 4H), 2.90-2.96 (m, 4H). LCMS found 586.1 [M+H]⁺.

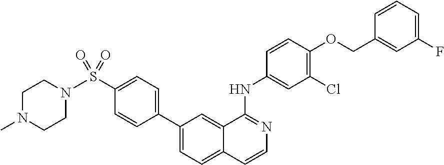 [SMILES: CN1CCN(S(=O)(=O)c2ccc(-c3ccc4ccnc(Nc5ccc(OCc6cccc(F)c6)c(Cl)c5)c4c3)cc2)CC1]

N-(3-Chloro-4-((3-fluorobenzyl)oxy)phenyl)-7-(4-((4-methylpiperazin-1-yl)sulfonyl)phenyl)isoquinolin-1-amine (57) General procedure A, FCC: ethyl acetate, isolated as a cream colored solid in 53% yield. ¹H NMR (500 MHz, CDCl₃) δ ppm 8.11-8.14 (m, 2H), 7.79-7.89 (m, 7H), 7.54 (dd, J=8.8, 2.4 Hz, 1H), 7.36 (td, J=7.8, 5.9 Hz, 1H), 7.28 (s, 1H), 7.21-7.27 (m, 2H), 7.18 (d, J=5.9 Hz, 1H), 7.03 (td, J=8.3, 2.4 Hz, 1H), 6.97 (d, J=8.8 Hz, 1H), 5.15 (s, 2H), 3.08 (br. s., 4H), 2.49 (t, J=4.6 Hz, 4H), 2.28 (s, 3H). LCMS found 617.2 [M+H]⁺.

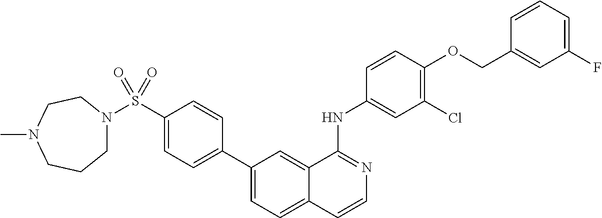 [SMILES: CN1CCCN(S(=O)(=O)c2ccc(-c3ccc4ccnc(Nc5ccc(OCc6cccc(F)c6)c(Cl)c5)c4c3)cc2)CC1]

N-(3-Chloro-4-((3-fluorobenzyl)oxy)phenyl)-7-(4-((4-methyl-1,4-diazepan-1-yl)sulfonyl)phenyl)isoquinolin-1-amine (59) General procedure A, FCC: 5% methanol in dichloromethane, isolated as an orange solid in 73% yield. ¹H NMR (500 MHz, DMSO-d₆) δ ppm 9.36 (s, 1H), 8.85 (s, 1H), 8.09-8.14 (m, 3H), 8.05 (d, J=2.4 Hz, 1H), 8.02 (d, J=5.9 Hz, 1H), 7.91-7.97 (m, 3H), 7.77 (dd, J=8.8, 2.9 Hz, 1H), 7.47 (td, J=8.1, 6.3 Hz, 1H), 7.29-7.36 (m, 2H), 7.24 (d, J=9.3 Hz, 1H), 7.22 (d, J=5.4 Hz, 1H), 7.18 (td, J=9.3, 2.0 Hz, 1H), 5.24 (s, 2H), 3.35-3.38 (m, 2H), 3.31-3.35 (m, 2H), 2.54-2.58 (m, 2H), 2.47-2.49 (m, 2H), 2.23 (s, 3H), 1.75 (quin, J=5.8 Hz, 2H). LCMS found 631.1 [M+H]⁺.

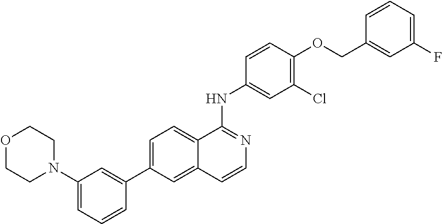 [SMILES: Fc1cccc(COc2ccc(Nc3nccc4cc(-c5cccc(N6CCOCC6)c5)ccc34)cc2Cl)c1]

N-(3-Chloro-4-((3-fluorobenzyl)oxy)phenyl)-6-(3-morpholinophenyl)isoquinolin-1-amine (54) General procedure A, FCC: 30-50% ethyl acetate in hexanes, isolated as an orange solid in 81% yield. ¹H NMR (500 MHz, DMSO-d₆) δ ppm 9.21 (s, 1H), 8.56 (d, J=8.8 Hz, 1H), 8.13 (d, J=2.4 Hz, 1H), 8.11 (d, J=2.0 Hz, 1H), 8.00 (d, J=5.4 Hz, 1H), 7.94 (dd, J=8.8, 1.5 Hz, 1H), 7.78 (dd, J=8.8, 2.4 Hz, 1H), 7.47 (td, J=8.0 , 6.3 Hz, 1H), 7.35-7.41 (m, 2H), 7.30-7.35 (m, 2H), 7.28 (d, J=7.8 Hz, 1H), 7.20-7.25 (m, 2H), 7.18 (td, J=8.8, 2.4 Hz, 1H), 7.03 (dd, J=8.1, 2.2 Hz, 1H), 5.23 (s, 2H), 3.76-3.81 (m, 4H), 3.21-3.25 (m, 4H). LCMS found 540.2 [M+H]⁺.

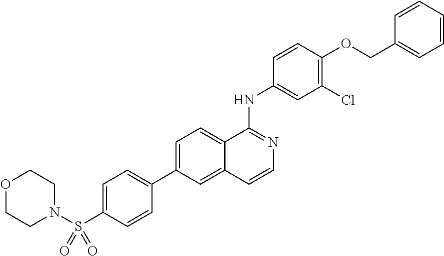 [SMILES: O=S(=O)(c1ccc(-c2ccc3c(Nc4ccc(OCc5ccccc5)c(Cl)c4)nccc3c2)cc1)N1CCOCC1]

N-(4-(Benzyloxy)-3-chlorophenyl)-6-(4-(morpholinosulfonyl)phenyl)isoquinolin-1-amine (56) General procedure A, FCC: 30-50% ethyl acetate in hexanes, isolated as an orange solid in 81% yield. ¹H NMR (500 MHz, DMSO-d₆) δ ppm 9.26 (s, 1H), 8.65 (d, J=9.3 Hz, 1H), 8.25 (d, J=1.5 Hz, 1H), 8.16 (d, J=8.8 Hz, 2H), 8.12 (d, J=2.9 Hz, 1H), 8.01-8.07 (m, 2H), 7.88 (d, J=8.3 Hz, 2H), 7.78 (dd, J=9.0, 2.7 Hz, 1H), 7.50 (d, J=6.8 Hz, 2H), 7.42 (t, J=7.3 Hz, 2H), 7.35 (t, J=7.3 Hz, 1H), 7.27 (d, J=5.9 Hz, 1H), 7.23 (d, J=9.3 Hz, 1H), 5.20 (s, 1H), 3.62-3.69 (m, 4H), 2.89-2.97 (m, 4H). LCMS found 586.1 [M+H]⁺.

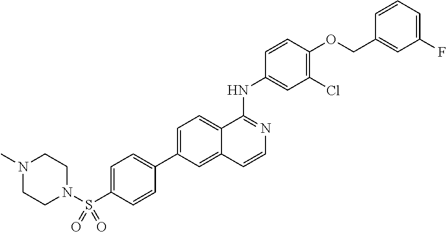 [SMILES: CN1CCN(S(=O)(=O)c2ccc(-c3ccc4c(Nc5ccc(OCc6cccc(F)c6)c(Cl)c5)nccc4c3)cc2)CC1]

N-(3-Chloro-4-((3-fluorobenzyl)oxy)phenyl)-6-(4-((4-methylpiperazin-1-yl)sulfonyl)phenyl)isoquinolin-1-amine (58) General procedure A, FCC: ethyl acetate, isolated as a pale orange solid in 52% yield. ¹H NMR (500 MHz, CDCl₃) δ ppm 8.14 (d, J=5.9 Hz, 1H), 8.02 (d, J=8.8 Hz, 1H), 7.95 (d, J=2.0 Hz, 1H), 7.83-7.91 (m, 5H), 7.76 (dd, J=8.5, 1.7 Hz, 1H), 7.50 (dd, J=8.8, 2.4 Hz, 1H), 7.37 (td, J=7.8, 5.9 Hz, 1H), 7.22-7.27 (m, 2H), 7.21 (d, J=5.9 Hz, 1H), 7.00-7.07 (m, 2H), 6.98 (d, J=8.8 Hz, 1H), 5.16 (s, 2H), 3.11 (br. s., 4H), 2.52 (t, J=4.6 Hz, 4H), 2.29 (s, 3H). LCMS found 617.2 [M+H]⁺.

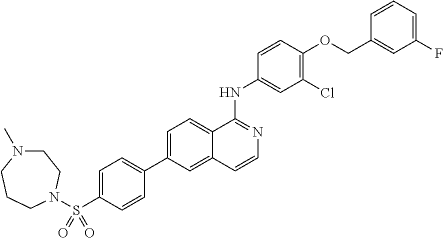 [SMILES: CN1CCCN(S(=O)(=O)c2ccc(-c3ccc4c(Nc5ccc(OCc6cccc(F)c6)c(Cl)c5)nccc4c3)cc2)CC1]

N-(3-Chloro-4-((3-fluorobenzyl)oxy)phenyl)-6-(4-((4-methyl-1,4-diazepan-1-yl)sulfonyl)phenyl)isoquinolin-1-amine (60) General procedure A, FCC: 5% methanol in dichloromethane, isolated as a pale yellow solid in 68% yield. ¹H NMR (500 MHz, DMSO-d₆) δ ppm 9.26 (s, 1H), 8.64 (d, J=8.8 Hz, 1H), 8.23 (d, J=1.5 Hz, 1H), 8.13 (d, J=2.4 Hz, 1H), 8.10 (d, J=8.3 Hz, 2H), 8.00-8.06 (m, 2H), 7.92 (d, J=8.3 Hz, 2H), 7.79 (dd, J=9.0, 2.7 Hz, 1H), 7.47 (td, J=8.3, 6.3 Hz, 1H), 7.29-7.35 (m, 2H), 7.27 (d, J=5.9 Hz, 1H), 7.22 (d, J=9.3 Hz, 1H), 7.18 (td, J=8.7, 2.7 Hz, 1H), 5.23 (s, 2H), 3.36 (m, J=4.9, 2.4, 2.4 Hz, 2H), 3.31-3.35 (m, 2H), 2.53-2.58 (m, 2H), 2.46-2.49 (m, 2H), 2.23 (s, 3H), 1.75 (quin, J=5.8 Hz, 2H). LCMS found 631.2 [M+H]⁺.

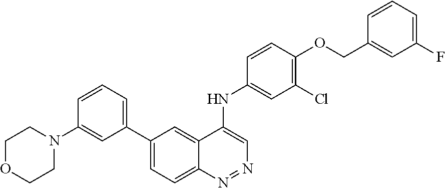 [SMILES: Fc1cccc(COc2ccc(Nc3cnnc4ccc(-c5cccc(N6CCOCC6)c5)cc34)cc2Cl)c1]

N-(3-Chloro-4-((3-fluorobenzyl)oxy)phenyl)-6-(3-morpholinophenyl)cinnolin-4-amine (61) General procedure B, FCC: 2-10% methanol in dichloromethane, isolated as a yellow solid in 67% yield. ¹H NMR (500 MHz, CDCl₃) δ 8.76 (br. s., 1H), 8.30 (s, 1H), 8.26 (d, J=4.9 Hz, 1H), 7.95 (dd, J=9.0, 1.2 Hz, 1H), 7.48-7.59 (m, 1H), 7.32-7.41 (m, 2H), 7.29 (t, J=7.8 Hz, 1H), 7.17-7.24 (m, 2H), 7.08-7.17 (m, 3H), 7.02 (td, J=8.4, 2.2 Hz, 1H), 6.87-6.94 (m, 2H), 5.13 (s, 2H), 3.76-3.82 (m, 4H), 3.09-3.16 (m, 4H). LCMS found 541.1 [M+H]⁺.

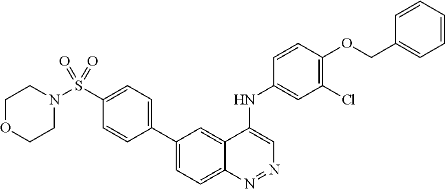 [SMILES: O=S(=O)(c1ccc(-c2ccc3nncc(Nc4ccc(OCc5ccccc5)c(Cl)c4)c3c2)cc1)N1CCOCC1]

N-(4-(Benzyloxy)-3-chlorophenyl)-6-(4-(morpholinosulfonyl)phenyl)cinnolin-4-amine (63) General procedure A, FCC: 2% methanol and 18% acetone in dichloromethane, then 1% methanol in ethyl acetate, isolated as a yellow solid in 8% yield. ¹H NMR (500 MHz, DMSO-d₆) δ 9.49 (br. s., 1H), 8.82 (br. s., 2H), 8.09-8.40 (m, 4H), 7.91 (d, J=7.8 Hz, 2H), 7.48-7.60 (m, 3H), 7.44 (t, J=7.6 Hz, 3H), 7.37 (t, J=7.3 Hz, 2H), 5.26 (s, 2H), 3.61-3.70 (m, 4H), 2.87-2.99 (m, 4H). LCMS found 587.1 [M+H]⁺.

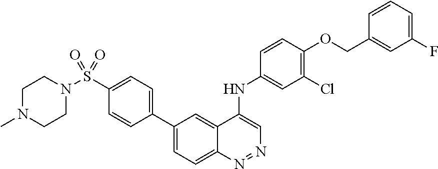 [SMILES: CN1CCN(S(=O)(=O)c2ccc(-c3ccc4nncc(Nc5ccc(OCc6cccc(F)c6)c(Cl)c5)c4c3)cc2)CC1]

N-(3-Chloro-4-((3-fluorobenzyl)oxy)phenyl)-6-(4-((4-methylpiperazin-1-yl)sulfonyl)phenyl)cinnolin-4-amine (65) General procedure A, FCC: 5-10% methanol in dichloromethane, then 10-30% methanol in ethyl acetate, isolated as a yellow solid in 15% yield.

¹H NMR (500 MHz, DMSO-d₆) δ 9.81 (s, 1H), 9.01 (s, 1H), 8.82 (s, 1H), 8.21-8.34 (m, 4H), 7.88 (d, J=8.3 Hz, 2H), 7.61 (d, J=2.0 Hz, 1H), 7.43-7.53 (m, 2H), 7.29-7.40 (m, 3H), 7.20 (t, J=8.8 Hz, 1H), 5.29 (s, 2H), 2.95 (br. s., 4H), 2.37 (br. s., 4H), 2.14 (s, 3H). LCMS found 618.1 [M+H]⁺.

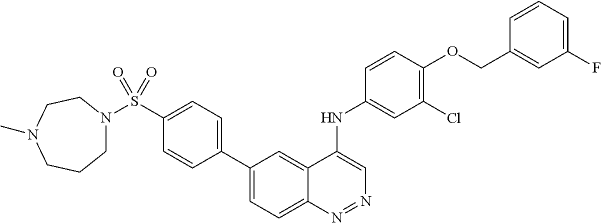 [SMILES: CN1CCCN(S(=O)(=O)c2ccc(-c3ccc4nncc(Nc5ccc(OCc6cccc(F)c6)c(Cl)c5)c4c3)cc2)CC1]

N-(3-Chloro-4-((3-fluorobenzyl)oxy)phenyl)-6-(4-((4-methyl-1,4-diazepan-1-yl)sulfonyl)phenyl)cinnolin-4-amine (67) General procedure B, FCC: 2-10% methanol in dichloromethane, isolated as a yellow solid in 61% yield. ¹H NMR (500 MHz, 1:1 CDCl₃/CD₃OD) 6 8.77 (br. s., 1H), 8.62 (s, 1H), 8.31 (br. s., 1H), 8.14 (d, J=8.8 Hz, 1H), 8.02 (d, J=8.3 Hz, 2H), 7.94 (d, J=8.8 Hz, 2H), 7.48 (br. s., 1H), 7.38-7.45 (m, 2H), 7.23-7.33 (m, 3H), 7.16 (d, J=8.8 Hz, 1H), 7.06 (td, J=8.4, 2.2 Hz, 1H), 5.25 (s, 2H), 3.47-3.50 (m, 2H), 3.45 (t, J=6.6 Hz, 2H), 2.70-2.74 (m, 2H), 2.66-2.70 (m, 2H), 2.38 (s, 3H), 1.89-1.96 (m, 2H). LCMS found 632.1 [M+H]⁺.

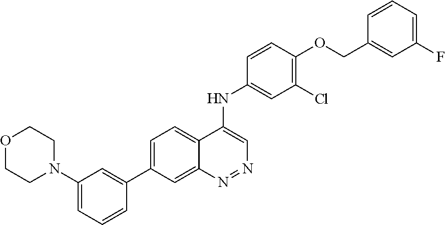 [SMILES: Fc1cccc(COc2ccc(Nc3cnnc4cc(-c5cccc(N6CCOCC6)c5)ccc34)cc2Cl)c1]

N-(3-Chloro-4-((3-fluorobenzyl)oxy)phenyl)-7-(3-morpholinophenyl)cinnolin-4-amine (62) General procedure B, FCC: 2-8% methanol in dichloromethane, then 50-100% ethyl acetate in hexanes, isolated as a yellow solid in 38% yield. ¹H NMR (500 MHz, METHANOL-d₄) δ 8.70 (br. s., 1H), 8.36 (br. s., 1H), 8.31 (d, J=8.8 Hz, 1H), 7.96 (d, J=8.8 Hz, 1H), 7.38-7.50 (m, 3H), 7.23-7.35 (m, 5H), 7.15 (d, J=8.8 Hz, 1H), 7.06 (m, J=8.3, 2.0 Hz, 2H), 5.24 (s, 2H), 3.90-3.96 (m, 4H), 3.27-3.32 (m, 4H). LCMS found 541.1 [M+H]⁺.

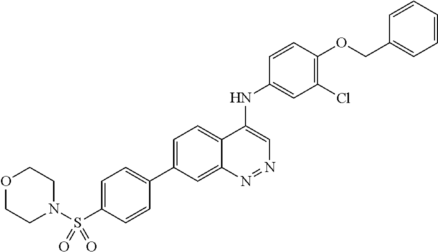 [SMILES: O=S(=O)(c1ccc(-c2ccc3c(Nc4ccc(OCc5ccccc5)c(Cl)c4)cnnc3c2)cc1)N1CCOCC1]

N-(4-(Benzyloxy)-3-chlorophenyl)-7-(4-(morpholinosulfonyl)phenyl)cinnolin-4-amine (64) General procedure B, FCC: 2-8% methanol in dichloromethane, then 10-100% ethyl acetate in dichloromethane, isolated as a yellow solid in 71% yield. ¹H NMR (500 MHz, METHANOL-d₄) δ 8.76 (br. s., 1H), 8.48 (br. s., 1H), 8.40 (d, J=8.8 Hz, 1H), 8.05 (d, J=8.3 Hz, 2H), 8.01 (d, J=8.8 Hz, 1H), 7.95 (d, J=8.3 Hz, 2H), 7.52 (d, J=7.3 Hz, 2H), 7.48 (br. s., 1H), 7.43 (t, J=7.6 Hz, 2H), 7.36 (t, J=7.3 Hz, 1H), 7.30 (d, J=8.8 Hz, 1H), 7.17 (d, J=8.8 Hz, 1H), 5.25 (s, 2H), 3.77-3.82 (m, 4H), 3.07-3.12 (m, 4H). LCMS found 587.1 [M+H]⁺.

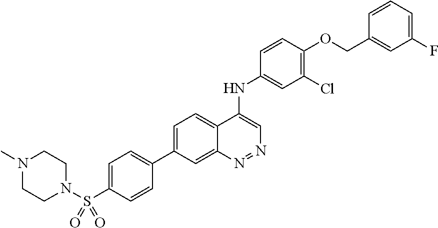 [SMILES: CN1CCN(S(=O)(=O)c2ccc(-c3ccc4c(Nc5ccc(OCc6cccc(F)c6)c(Cl)c5)cnnc4c3)cc2)CC1]

N-(3-Chloro-4-((3-fluorobenzyl)oxy)phenyl)-7-(4-((4-methylpiperazin-1-yl)sulfonyl)phenyl)cinnolin-4-amine (66) General procedure B, FCC: 5-10% methanol in dichloromethane, isolated as a yellow solid in 50% yield. ¹H NMR (500 MHz, METHANOL-d₄) δ 8.74 (br. s., 1H), 8.44 (br. s., 1H), 8.39 (d, J=8.8 Hz, 1H), 7.93-8.04 (m, 5H), 7.48 (br. s., 1H), 7.42 (td, J=8.1, 5.9 Hz, 1H), 7.31 (d, J=7.8 Hz, 2H), 7.26 (d, J=9.8 Hz, 1H), 7.16 (d, J=8.8 Hz, 1H), 7.06 (td, J=8.5, 2.0 Hz, 1H), 5.25 (s, 2H), 3.48-3.52 (m, 2H), 3.46 (t, J=6.3 Hz, 2H), 2.73-2.77 (m, 2H), 2.69-2.73 (m, 2H), 2.40 (s, 3H), 1.95 (dt, J=11.4, 5.8 Hz, 2H). LCMS found 618.1 [M+H]⁺.

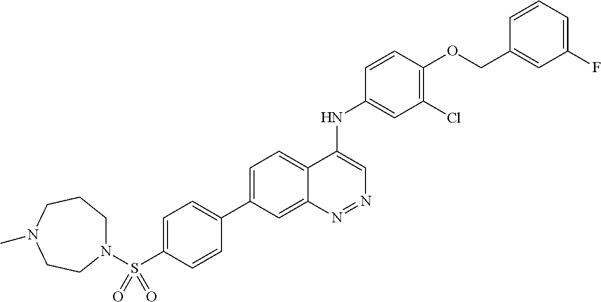 [SMILES: CN1CCCN(S(=O)(=O)c2ccc(-c3ccc4c(Nc5ccc(OCc6cccc(F)c6)c(Cl)c5)cnnc4c3)cc2)CC1]

N-(3-Chloro-4-((3-fluorobenzyl)oxy)phenyl)-7-(4-((4-methyl-1,4-diazepan-1-yl)sulfonyl)phenyl)cinnolin-4-amine (68) General procedure B, FCC: 5-15% methanol in dichloromethane, isolated as a yellow solid in 52% yield. ¹H NMR (500 MHz, METHANOL-d₄) δ 8.74 (br. s., 1H), 8.44 (br. s., 1H), 8.39 (d, J=8.8 Hz, 1H), 7.93-8.04 (m, 5H), 7.48 (br. s., 1H), 7.42 (td, J=8.1, 5.9 Hz, 1H), 7.31 (d, J=7.8 Hz, 2H), 7.26 (d, J=9.8 Hz, 1H), 7.16 (d, J=8.8 Hz, 1H), 7.06 (td, J=8.5, 2.0 Hz, 1H), 5.25 (s, 2H), 3.48-3.52 (m, 2H), 3.46 (t, J=6.3 Hz, 2H), 2.73-2.77 (m, 2H), 2.69-2.73 (m, 2H), 2.40 (s, 3H), 1.95 (dt, J=11.4, 5.8 Hz, 2H). LCMS found 632.1 [M+H]⁺.

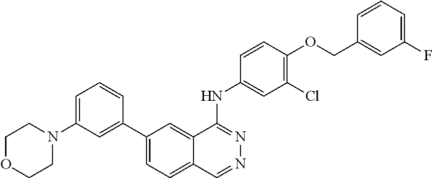 [SMILES: Fc1cccc(COc2ccc(Nc3nncc4ccc(-c5cccc(N6CCOCC6)c5)cc34)cc2Cl)c1]

N-(3-Chloro-4-((3-fluorobenzyl)oxy)phenyl)-7-(3-morpholinophenyl)phthalazin-1-amine (69) General procedure C, FCC: 40-80% ethyl acetate in hexanes, then prep HPLC 30-50% acetonitrile in water, isolated as a yellow solid in 9% yield. ¹H NMR (500 MHz, DMSO-d₆) δ 9.27 (s, 1H), 9.14 (s, 1H), 8.78 (s, 1H), 8.27 (dd, J=8.3, 1.5 Hz, 1H), 8.16 (d, J=2.4 Hz, 1H), 8.09 (d, J=8.3 Hz, 1H), 7.82 (dd, J=9.0, 2.7 Hz, 1H), 7.40-7.50 (m, 2H), 7.39 (s, 1H), 7.30-7.36 (m, 3H), 7.27 (d, J=8.8 Hz, 1H), 7.18 (td, J=8.7, 2.2 Hz, 1H), 7.07 (dd, J=8.3, 2.0 Hz, 1H), 5.25 (s, 2H), 3.76-3.82 (m, 4H), 3.22-3.27 (m, 4H). LCMS found 541.2 [M+H]⁺.

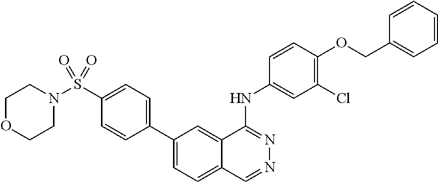 [SMILES: O=S(=O)(c1ccc(-c2ccc3cnnc(Nc4ccc(OCc5ccccc5)c(Cl)c4)c3c2)cc1)N1CCOCC1]

N-(4-(Benzyloxy)-3-chlorophenyl)-7-(4-(morpholinosulfonyl)phenyl)phthalazin-1-amine (71) General procedure C, FCC: 50-100% ethyl acetate in hexanes, then prep HPLC 30-50% acetonitrile in water, isolated as a yellow solid in 9% yield. ¹H NMR (500 MHz, DMSO-d₆) δ 9.36 (s, 1H), 9.18 (s, 1H), 8.93 (s, 1H), 8.36 (dd, J=8.3, 1.5 Hz, 1H), 8.20 (d, J=8.3 Hz, 2H), 8.17 (d, J=8.3 Hz, 1H), 8.15 (d, J=2.9 Hz, 1H), 7.94 (d, J=8.3 Hz, 2H), 7.82 (dd, J=9.0, 2.7 Hz, 1H), 7.50 (d, J=7.3 Hz, 2H), 7.42 (t, J=7.6 Hz, 2H), 7.35 (t, J=7.3 Hz, 1H), 7.30 (d, J=9.3 Hz, 1H), 5.22 (s, 2H), 3.63-3.69 (m, 4H), 2.90-2.98 (m, 4H). LCMS found 587.2 [M+H]⁺.

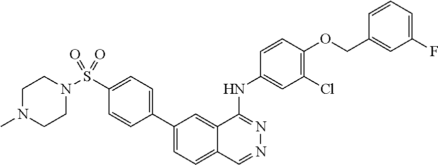 [SMILES: CN1CCN(S(=O)(=O)c2ccc(-c3ccc4cnnc(Nc5ccc(OCc6cccc(F)c6)c(Cl)c5)c4c3)cc2)CC1]

N-(3-Chloro-4-((3-fluorobenzyl)oxy)phenyl)-7-(4-((4-methylpiperazin-1-yl)sulfonyl)phenyl)phthalazin-1-amine, Formic Acid (73) General procedure B, FCC: 3-7% methanol in dichloromethane, then prep HPLC 5-95% acetonitrile in water, isolated as an orange solid in 39% yield. ¹H NMR (500 MHz, DMSO-d₆) δ 9.32 (br. s., 1H), 9.18 (s, 1H), 8.91 (s, 1H), 8.35 (d, J=7.3 Hz, 1H), 8.12-8.21 (m, 6H), 7.93 (d, J=8.3 Hz, 2H), 7.83 (d, J=6.8 Hz, 1H), 7.47 (td, J=8.3 , 6.3 Hz, 1H), 7.30-7.36 (m, 2H), 7.28 (d, J=8.8 Hz, 1H), 7.18 (td, J=8.7, 2.2 Hz, 1H), 5.25 (s, 2H), 2.96 (br. s., 4H), 2.35-2.43 (m, 4H), 2.15 (s, 3H). LCMS found 618.2 [M+H]⁺.

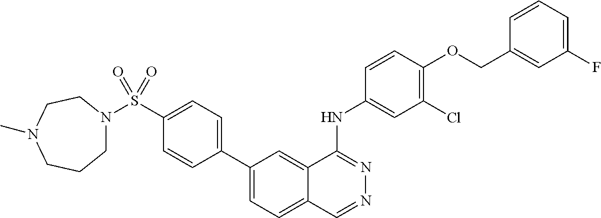 [SMILES: CN1CCCN(S(=O)(=O)c2ccc(-c3ccc4cnnc(Nc5ccc(OCc6cccc(F)c6)c(Cl)c5)c4c3)cc2)CC1]

N-(3-Chloro-4-((3-fluorobenzyl)oxy)phenyl)-7-(4-((4-methyl-1,4-diazepan-1-yl)sulfonyl)phenyl)phthalazin-1-amine, Formic Acid (75) General procedure B, FCC: 5-10% methanol in dichloromethane, then prep HPLC 5-95% acetonitrile in water, isolated as an orange solid in 29% yield. ¹H NMR (500 MHz, METHANOL-d₄) δ 8.94 (s, 1H), 8.73 (s, 1H), 8.25 (s, 2H), 8.20 (dd, J=8.5, 1.2 Hz, 1H), 8.00-8.06 (m, 3H), 7.95 (d, J=8.3 Hz, 2H), 7.88 (d, J=2.4 Hz, 1H), 7.59 (dd, J=8.8, 2.4 Hz, 1H), 7.38 (td, J=8.1, 5.9 Hz, 1H), 7.28 (d, J=7.8 Hz, 1H), 7.23 (d, J=9.8 Hz, 1H), 6.99-7.06 (m, 2H), 5.18 (s, 2H), 3.60-3.66 (m, 2H), 3.47 (t, J=6.6 Hz, 2H), 3.22-3.28 (m, 4H), 2.77 (s, 3H), 2.14-2.21 (m, 2H). LCMS found 632.2 [M+H]⁺.

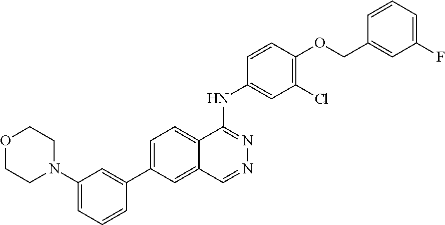 [SMILES: Fc1cccc(COc2ccc(Nc3nncc4cc(-c5cccc(N6CCOCC6)c5)ccc34)cc2Cl)c1]

N-(3-Chloro-4-((3-fluorobenzyl)oxy)phenyl)-6-(3-morpholinophenyl)phthalazin-1-amine (70), General procedure C, FCC: 40-80% ethyl acetate in hexanes, then prep HPLC 30-50% acetonitrile in water, isolated as a pale yellow solid in 14% yield. ¹H NMR (500 MHz, DMSO-d₆) δ 9.24 (s, 1H), 9.16 (s, 1H), 8.62 (d, J=8.3 Hz, 1H), 8.30-8.36 (m, 2H), 8.21 (d, J=2.4 Hz, 1H), 7.84 (dd, J=9.0, 2.7 Hz, 1H), 7.47 (td, J=7.8, 5.9 Hz, 1H), 7.38-7.43 (m, 2H), 7.29-7.36 (m, 3H), 7.26 (d, J=9.3 Hz, 1H), 7.18 (td, J=8.7, 2.2 Hz, 1H), 7.06 (dd, J=8.3, 2.0 Hz, 1H), 5.25 (s, 2H), 3.75-3.83 (m, 4H), 3.21-3.28 (m, 4H). LCMS found 541.2 [M+H]⁺.

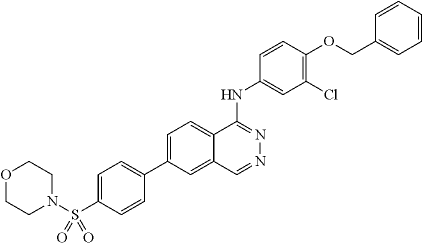 [SMILES: O=S(=O)(c1ccc(-c2ccc3c(Nc4ccc(OCc5ccccc5)c(Cl)c4)nncc3c2)cc1)N1CCOCC1]

N-(4-(Benzyloxy)-3-chlorophenyl)-6-(4-(morpholinosulfonyl)phenyl)phthalazin-1-amine (72) General procedure C, FCC: 5-100% ethyl acetate in hexanes, then prep HPLC 30-50% acetonitrile in water, isolated as a yellow solid in 23% yield. ¹H NMR (500 MHz, DMSO-d₆) δ 9.28 (s, 1H), 9.19 (s, 1H), 8.70 (d, J=8.8 Hz, 1H), 8.45 (d, J=2.0 Hz, 1H), 8.41 (dd, J=8.5, 1.7 Hz, 1H), 8.20 (d, J=2.4 Hz, 1H), 8.18 (d, J=8.3 Hz, 2H), 7.91 (d, J=8.3 Hz, 2H), 7.83 (dd, J=8.8, 2.4 Hz, 1H), 7.50 (d, J=6.8 Hz, 2H), 7.42 (t, J=7.3 Hz, 2H), 7.35 (t, J=6.8 Hz, 1H), 7.27 (d, J=8.8 Hz, 1H), 5.21 (s, 2H), 3.63-3.69 (m, 4H), 2.91-2.97 (m, 4H). LCMS found 587.1 [M+H]⁺.

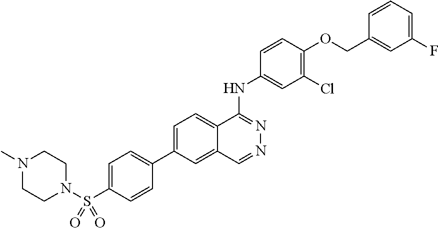 [SMILES: CN1CCN(S(=O)(=O)c2ccc(-c3ccc4c(Nc5ccc(OCc6cccc(F)c6)c(Cl)c5)nncc4c3)cc2)CC1]

N-(3-Chloro-4-((3-fluorobenzyl)oxy)phenyl)-6-(4-((4-methylpiperazin-1-yl)sulfonyl)phenyl)phthalazin-1-amine, Formic Acid (74) General procedure B, FCC: 3-10% methanol in dichloromethane, then prep HPLC 5-95% acetonitrile in water, isolated as a dull yellow solid in 28% yield. ¹H NMR (500 MHz, DMSO-d₆) δ 9.28 (s, 1H), 9.20 (s, 1H), 8.70 (d, J=8.3 Hz, 1H), 8.47 (d, J=2.0 Hz, 1H), 8.42 (dd, J=8.5, 1.7 Hz, 1H), 8.21 (d, J=2.9 Hz, 1H), 8.16-8.20 (m, 3H), 7.91 (d, J=8.3 Hz, 2H), 7.84 (dd, J=9.0, 2.7 Hz, 1H), 7.48 (td, J=8.3 , 6.3 Hz, 1H), 7.30-7.36 (m, 2H), 7.27 (d, J=9.3 Hz, 1H), 7.18 (td, J=8.7, 2.7 Hz, 1H), 5.25 (s, 2H), 2.96 (br. s., 4H), 2.39 (t, J=4.6 Hz, 4H), 2.15 (s, 3H). LCMS found 618.2 [M+H]⁺.

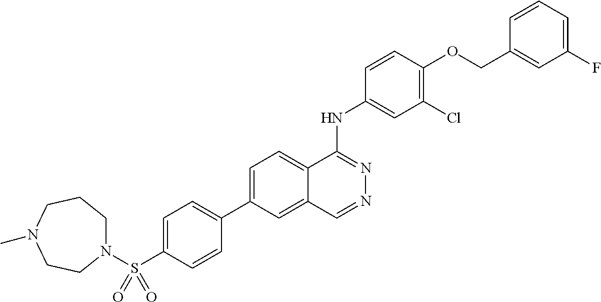 [SMILES: CN1CCCN(S(=O)(=O)c2ccc(-c3ccc4c(Nc5ccc(OCc6cccc(F)c6)c(Cl)c5)nncc4c3)cc2)CC1]

N-(3-Chloro-4-((3-fluorobenzyl)oxy)phenyl)-6-(4-((4-methyl-1,4-diazepan-1-yl)sulfonyl)phenyl)phthalazin-1-amine, Formic Acid (76) General procedure B, FCC: 5-15% methanol in dichloromethane, then prep HPLC 5-95% acetonitrile in water, isolated as a light yellow solid in 29% yield. ¹H NMR (500 MHz, METHANOL-d₄) δ 8.99 (s, 1H), 8.51 (d, J=8.3 Hz, 1H), 8.25 (s, 1H), 8.17-8.22 (m, 2H), 7.97 (s, 4H), 7.91 (d, J=2.9 Hz, 1H), 7.58-7.63 (m, 1H), 7.39 (td, J=7.8, 5.9 Hz, 1H), 7.29 (d, J=7.8 Hz, 1H), 7.24 (d, J=9.3 Hz, 1H), 7.00-7.08 (m, 2H), 5.20 (s, 2H), 3.54-3.59 (m, 2H), 3.47 (t, J=6.6 Hz, 2H), 2.97-3.04 (m, 4H), 2.60 (s, 3H), 2.06 (quin, J=6.3 Hz, 2H). LCMS found 632.2 [M+H]⁺.

The invention being thus described, it will be apparent that the same may be varied in many ways. Such variations are not to be regarded as a departure from the spirit and scope of the invention, and all such modifications as would be recognized by one skilled in the art are intended to be included within the scope of the following claims.

What is claimed is:

1. A compound represented by the following structure: 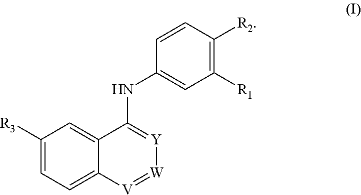
wherein
V, W and Y are independently CH or N with at least one of V, W and Y being N;
R₁ is hydrogen, halogen or —O((C₁-C₆)-alkyl);
R₂ is hydrogen, —(C₁-C₆)-alkyl, —OR₄; or R₁ and R₂ together form a 3 to 8-membered heterocycle, wherein at least one of the ring carbon atoms is optionally replaced with a heteroatom, selected from N, O and S and wherein the heterocycle is optionally substituted;
R₃ is substituted or unsubstituted 6 member aryl or heterocycle; and
R₄ is H, —(C₁-C₆)-alkyl, benzyl, substituted benzyl, halo-, dihalo-, or trihalo benzyl, methoxybenzyl;
or a pharmaceutically acceptable salt thereof.

2. The compound of claim 1 represented by the following structure: 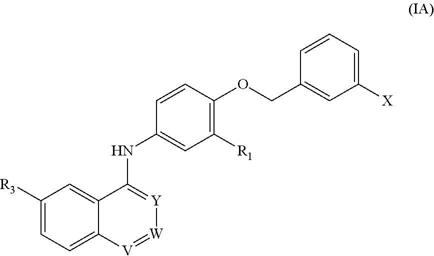
wherein X is hydrogen or halogen.

3. The compound of claim 2 represented by the following structure: 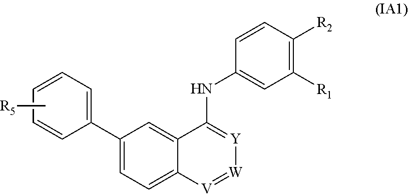
wherein R₅ is hydrogen, —(C₁-C₆)-alkyl, —(C₃-C₅)-cycloalkyl, —C(O)R₆, or —S(O)₂R₆, and R₆ is —(C₁-C₆)-alkyl, aminoalkyl, of a 3 to 8-membered heterocycle, wherein at least one of the ring carbon atoms is optionally replaced with a heteroatom selected from the group N, O and S, and wherein the heterocycle is optionally substituted with —(C₁-C₆)-alkyl.

4. The compound of claim 3 represented by the following structure:

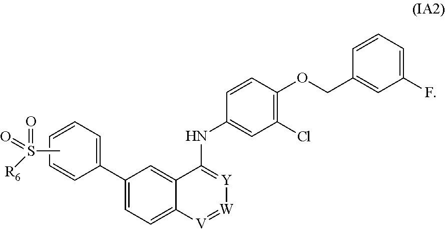

5. The compound of claim 4 wherein R₆ is CH₃;

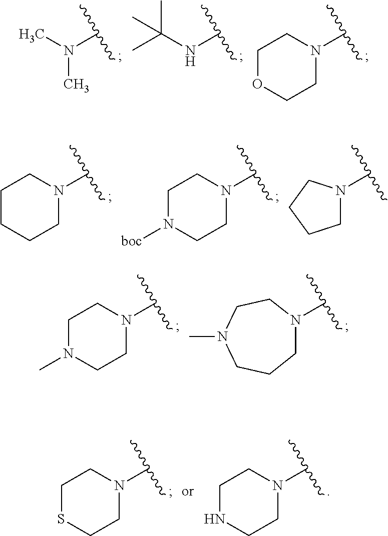

6. The compound of claim 3 represented by the following structure:

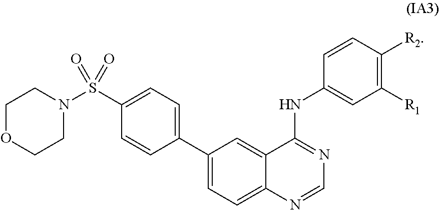

7. The compound of claim 6 wherein R₁ is CH₃; OH; Obn; OCH₃; 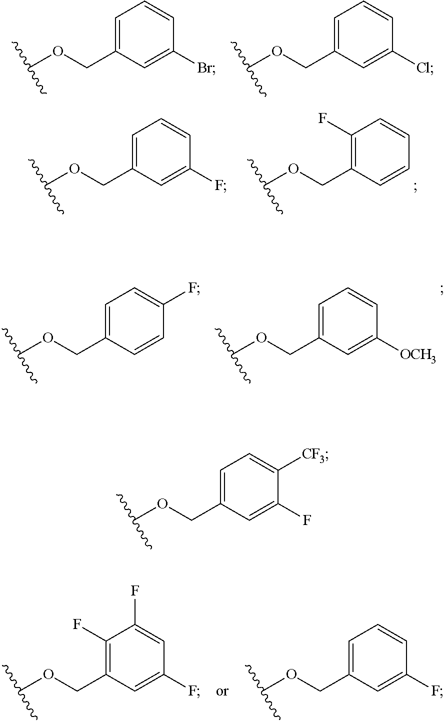
and R₂ is hydrogen, Cl, or methoxy.

8. The compound of claim 3 represented by the following structure:

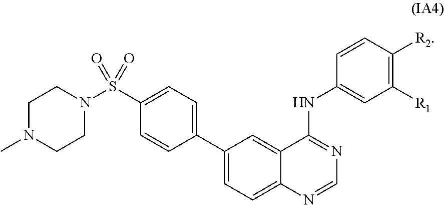

9. The compound of claim 2 represented by the following structure:

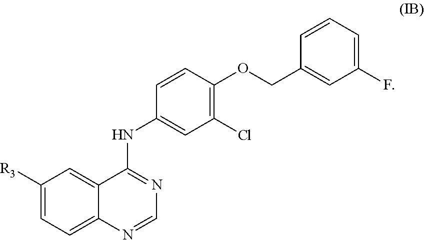

10. The compound of claim 2 represented by the following structure:

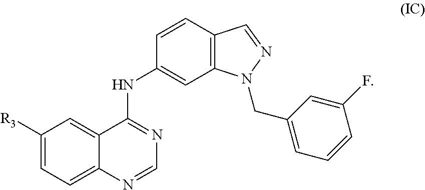

11. The compound of claim 10 wherein R₃ is:

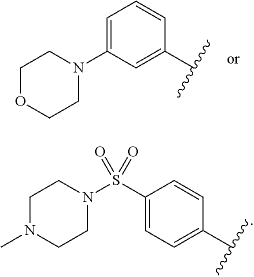 [SMILES: *c1cccc(N2CCOCC2)c1 ; *c1ccc(S(=O)(=O)N2CCN(C)CC2)cc1]

12. The compound of claim 2 wherein the compound is:

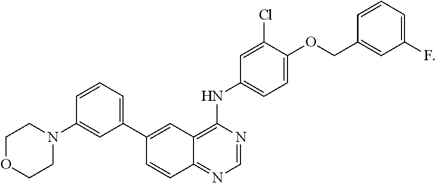 [SMILES: Fc1cccc(COc2ccc(Nc3ncnc4ccc(-c5cccc(N6CCOCC6)c5)cc34)cc2Cl)c1]

13. A composition comprising a compound of claim 1 and a pharmaceutically acceptable carrier.

14. A method of treating protozoan parasite infection in a subject comprising administration of a therapeutically effective amount of a compound of claim 1.

15. The method of claim 14 wherein the protozoan parasite is selected from the group consisting of Trypanosoma brucei, Trypanosoma cruzi, Leishmania spp., and Plasmodium spp.

16. A method for inhibiting growth of a protozoan parasite comprising contacting said protozoan parasite with a compound of claim 1.

17. A compound represented by the following structure: 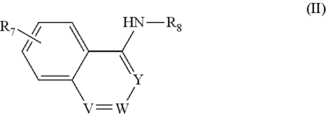
wherein
V, W and Y are independently CH or N, wherein at least 1 of V, W and Y is N;
R₇ is substituted or unsubstituted aryl; and
R8 is substituted or unsubstituted aryl or 5 to 6-membered heterocycle, wherein at least one of the ring carbon atoms is optionally replaced with a heteroatom, selected from N, O and S and wherein the heterocycle is optionally substituted,
or a pharmaceutically acceptable salt thereof.

18. The compound of claim 17 represented by the following structure: 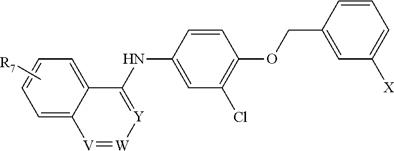
wherein X is hydrogen or halogen.

19. The compound of claim 17 wherein V is N and W and Y are each CH.

20. The compound of claim 17 wherein Y is N.

21. The compound of claim 17 wherein W is N.

22. The compound of claim 18 wherein R₇ is

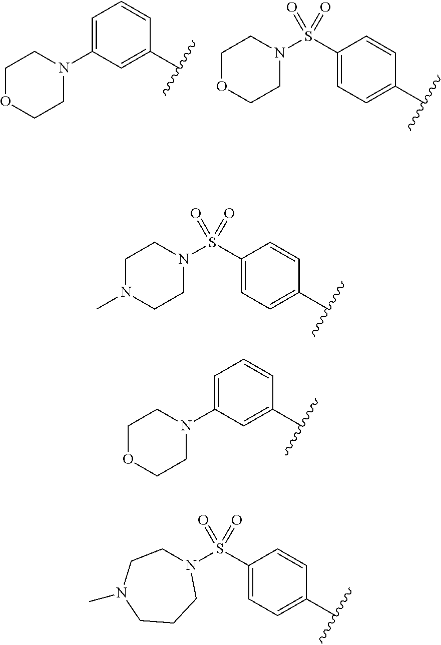

23. The compound of claim 17 wherein R₈ is a substituted phenyl.
